SEQUENCE LISTING (33 sequences)

SEQ ID NO: 1 - DNA, 21 bp, Artificial Sequence
  Synthetic polynucleotide
cacgtgcgcagcaggacgcag

SEQ ID NO: 2 - DNA, 24 bp, Artificial Sequence
  Synthetic polynucleotide
gccaacatgacttacttgatcccc

SEQ ID NO: 3 - DNA, 25 bp, Artificial Sequence
  Synthetic polynucleotide
gccaacatgacttacttgatcccca

SEQ ID NO: 4 - DNA, 20 bp, Artificial Sequence
  Synthetic polynucleotide
aatcgaccggtggtaaagca

SEQ ID NO: 5 - DNA, 30 bp, Artificial Sequence
  Synthetic polynucleotide
cagacaactgttcaaactgatgggacccac

SEQ ID NO: 6 - DNA, 21 bp, Artificial Sequence
  Synthetic polynucleotide
tgggggttctggggactggct

SEQ ID NO: 7 - DNA, 18 bp, Artificial Sequence
  Synthetic polynucleotide
  misc_feature (1)..(1): modified by FAM
  misc_feature (9)..(9): modified by locked nucleic acids
  misc_feature (10)..(10): modified by locked nucleic acids
  misc_feature (18)..(18): modified by 3BHQ
ccagccccttccgggccc

SEQ ID NO: 8 - DNA, 18 bp, Artificial Sequence
  Synthetic polynucleotide
  misc_feature (1)..(1): modified by FAM
  misc_feature (8)..(10): modified by locked nucleic acids
  misc_feature (18)..(18): modified by 3BHQ
cgaccccttccgggtccc

SEQ ID NO: 9 - DNA, 15 bp, Artificial Sequence
  Synthetic polynucleotide
  misc_feature (1)..(1): modified by FAM
  misc_feature (3)..(8): modified by locked nucleic acids
  misc_feature (11)..(13): modified by locked nucleic acids
  misc_feature (15)..(15): modified by Dab
aggtcatcatgctta

SEQ ID NO: 10 - DNA, 15 bp, Artificial Sequence
  Synthetic polynucleotide
  misc_feature (1)..(1): modified by MAXN
  misc_feature (3)..(9): modified by locked nucleic acids
  misc_feature (11)..(12): modified by locked nucleic acids
  misc_feature (15)..(15): modified by Dab
aggttgtcatgctta

SEQ ID NO: 11 - DNA, 15 bp, Artificial Sequence
  Synthetic polynucleotide
  misc_feature (1)..(1): modified by MAXN
  misc_feature (5)..(8): modified by locked nucleic acids
  misc_feature (11)..(11): modified by locked nucleic acids
  misc_feature (13)..(14): modified by locked nucleic acids
  misc_feature (15)..(15): modified by Dab
aggtggtcatgctta

SEQ ID NO: 12 - DNA, 15 bp, Artificial Sequence
  Synthetic polynucleotide
  misc_feature (1)..(1): modified by MAXN
  misc_feature (3)..(3): modified by locked nucleic acids
  misc_feature (5)..(8): modified by locked nucleic acids
  misc_feature (10)..(12): modified by locked nucleic acids
  misc_feature (14)..(14): modified by locked nucleic acids
  misc_feature (15)..(15): modified by Dab
aggtagtcatgctta

SEQ ID NO: 13 - DNA, 15 bp, Artificial Sequence
  Synthetic polynucleotide
  misc_feature (1)..(1): modified by MAXN
  misc_feature (3)..(8): modified by locked nucleic acids
  misc_feature (11)..(13): modified by locked nucleic acids
  misc_feature (15)..(15): modified by Dab
aggtcttcatgctta

SEQ ID NO: 14 - DNA, 13 bp, Artificial Sequence
  Synthetic polynucleotide
  misc_feature (1)..(1): modified by FAM
  misc_feature (3)..(4): modified by locked nucleic acids
  misc_feature (6)..(7): modified by locked nucleic acids
  misc_feature (9)..(9): modified by locked nucleic acids
  misc_feature (11)..(11): modified by locked nucleic acids
  misc_feature (13)..(13): modified by Dab
ctcgcatgagtgc

SEQ ID NO: 15 - DNA, 20 bp, Artificial Sequence
  Synthetic polynucleotide
  misc_feature (1)..(1): modified by FAM
  misc_feature (4)..(5): modified by locked nucleic acids
  misc_feature (8)..(8): modified by locked nucleic acids
  misc_feature (10)..(12): modified by locked nucleic acids
  misc_feature (17)..(17): modified by locked nucleic acids
  misc_feature (20)..(20): modified by Dab
ctagctacagagaaatctcg

SEQ ID NO: 16 - DNA, 21 bp, Artificial Sequence
  Synthetic polynucleotide
  misc_feature (1)..(1): modified by Cy5
  misc_feature (5)..(5): modified by locked nucleic acids
  misc_feature (8)..(8): modified by locked nucleic acids
  misc_feature (11)..(11): modified by locked nucleic acids
  misc_feature (13)..(13): modified by locked nucleic acids
  misc_feature (18)..(18): modified by locked nucleic acids
  misc_feature (20)..(20): modified by locked nucleic acids
  misc_feature (21)..(21): modified by IowaBlack
cacagtccatgccatcactgc

SEQ ID NO: 17 - DNA, 22 bp, Artificial Sequence
  Synthetic polynucleotide
cttcaccttccagctccgcctc

SEQ ID NO: 18 - DNA, 21 bp, Artificial Sequence
  Synthetic polynucleotide
gcacggtcttcagagaagcca

SEQ ID NO: 19 - DNA, 24 bp, Artificial Sequence
  Synthetic polynucleotide
acatacaagagagactttgtccca

SEQ ID NO: 20 - DNA, 17 bp, Artificial Sequence
  Synthetic polynucleotide
  misc_feature (2)..(2): modified by thymine bases with 5-hydroxybutynyl-2′-deoxyuridine to stabilize oligonucleotide hybridization
  misc_feature (4)..(4): modified by thymine bases with 5-hydroxybutynyl-2′-deoxyuridine to stabilize oligonucleotide hybridization
  misc_feature (7)..(7): modified by thymine bases with 5-hydroxybutynyl-2′-deoxyuridine to stabilize oligonucleotide hybridization
  misc_feature (15)..(15): modified by thymine bases with 5-hydroxybutynyl-2′-deoxyuridine to stabilize oligonucleotide hybridization
ctctgataggaaaatga

SEQ ID NO: 21 - DNA, 22 bp, Artificial Sequence
  Synthetic polynucleotide
atgatgttctggagagccccgc

SEQ ID NO: 22 - DNA, 19 bp, Artificial Sequence
  Synthetic polynucleotide
cccagccccctccgggccc

SEQ ID NO: 23 - DNA, 21 bp, Artificial Sequence
  Synthetic polynucleotide
cccgacccctcccgggtcccc

SEQ ID NO: 24 - DNA, 18 bp, Artificial Sequence
  Synthetic polynucleotide
aggtcgtcatgcttatgg

SEQ ID NO: 25 - DNA, 15 bp, Artificial Sequence
  Synthetic polynucleotide
gctcgcaagagtgcg

SEQ ID NO: 26 - DNA, 20 bp, Artificial Sequence
  Synthetic polynucleotide
ctagctacagtgaaatctcg

SEQ ID NO: 27 - DNA, 10 bp, Artificial Sequence
  Synthetic polynucleotide
gccccctccg

SEQ ID NO: 28 - DNA, 10 bp, Artificial Sequence
  Synthetic polynucleotide
gccccttccg

SEQ ID NO: 29 - DNA, 10 bp, Artificial Sequence
  Synthetic polynucleotide
cccctcccgg

SEQ ID NO: 30 - DNA, 10 bp, Artificial Sequence
  Synthetic polynucleotide
ccccttccgg

SEQ ID NO: 31 - DNA, 10 bp, Artificial Sequence
  Synthetic polynucleotide
gcaagagtgc

SEQ ID NO: 32 - DNA, 11 bp, Artificial Sequence
  Synthetic polynucleotide
gcatgagtgcg

SEQ ID NO: 33 - DNA, 11 bp, Artificial Sequence
  Synthetic polynucleotide
ctacagtgaaa

(19) United States
(12) Patent Application Publication
(10) Pub. No.: US 2019/0160139 A1
(43) Pub. Date: May 30, 2019

(54) GENOTYPE-DIRECTED LOCAL DELIVERY OF TARGETED THERAPEUTICS

(71) Applicants: Massachusetts Institute of Technology, Cambridge, MA (US); The General Hospital Corporation, Boston, MA (US); The Brigham and Women's Hospital, Inc., Boston, MA (US)

(72) Inventors: Robert S. Langer, Newton, MA (US); Carlo Giovanni Traverso, Newton, MA (US); Ameya R. Kirtane, Quincy, MA (US); Daniel Cahill, Winchester, MA (US); Ganesh Shankar, Brookline, MA (US)

(21) Appl. No.: 16/119,135

(22) Filed: Aug. 31, 2018

Related U.S. Application Data
(60) Provisional application No. 62/558,189, filed on Sep. 13, 2017.

(51) Int. Cl. A61K 38/00; A61K 9/00; A61K 9/51; A61P 35/00; C12Q 1/6858; C12Q 1/6886; C12N 9/04; C12N 9/10

(52) U.S. Cl. CPC A61K 38/005; A61K 9/0019; A61K 9/51; A61P 35/00; C12Y 204/02012; C12Q 1/6886; C12N 9/0006; C12N 9/1077; C12Y 101/01041; C12Q 1/6858

(57) ABSTRACT

Provided herein are pharmaceutical compositions for local administration of metabolic inhibitors, methods of locally administering such compositions, and rapid diagnostic methods for identifying mutant allele during the course of a surgical procedure.

### RELATED APPLICATION

This application claims the benefit under 35 U.S.C. § 119(e) of U.S. provisional application Ser. No. 62/558,189, filed Sep. 13, 2017, which is incorporated by reference herein in its entirety.

### GOVERNMENT FUNDING

This invention was made with Government funding support under Grant No. R37-EB000244 awarded by the National Institutes of Health. The Government has certain rights in this invention.

### FIELD OF THE INVENTION

The present disclosure relates to pharmaceutical compositions for the treatment of cancer, methods of locally administering the compositions, and rapid diagnostic methods for identifying subjects for local administration of the compositions.

### BACKGROUND

Genotype-based precision therapeutics have had a transformative impact in medical oncology. However, surgical oncology has lagged in the application of molecular therapeutics due to the difficulty in obtaining actionable tumor molecular diagnostic information during the accelerated timeframe required for intraoperative decision-making. Additionally, there has been a perceived lack-of-necessity for administering such therapeutics during surgery, when the therapeutics can be given systemically instead. However, despite their potential efficacy in inhibiting or killing cancer cells, some targeted therapeutics cannot be dosed systemically, due to toxicity or poor distribution into privileged sites such as the central nervous system (CNS), behind the blood-brain barrier.

The need for this diagnostic information during surgery is becoming increasingly evident. The utility of local chemotherapeutic administration for high-grade glioma has been long recognized, with the design of polymeric carmustine wafers for implantation into the resection cavity of high-grade gliomas (Brem et al. Lancet (1995) 345(8956) 1008-12; Moses et al. Cancer Cell (2003) 4(5): 337-41). The therapeutic impact of surgical resectioning and the durability of local control at the primary tumor site varies substantially amongst different molecular subgroups. For example, surgical resectioning can be curative for BRAF mutant lesions characterized by ganglioglioma or pleomorphic xanthoastrocytoma histology. Patients with IDH1 mutant diffuse gliomas (approximately 25% of cases) also experienced substantially prolonged survival with aggressive local surgical resection (Beiko et al. Neuro. Oncol. (2014) 16: 81-91; Kawaguchi et al. J. Neurooncol. (2016) 129: 505-514) and/or focal radiation therapy followed by chemotherapy (Caincross et al. J. Clin. Oncol. Off. J. Am. Soc. Clin. Oncol.I (2014) 32:783-790; Buckner et al. N. Engl. J. Med. (2016) 1344-1355; Baumert et al. Lancet Oncol. (2016) 17: 1521-1532). This local control is often not long-term in IDH1 mutant gliomas, as cancer cells diffusely infiltrate alongside normal cells at the tumor margins, and recurrence is common.

### SUMMARY OF THE INVENTION

Aspects of the present disclosure provide pharmaceutical compositions comprising a population of particles coupled to a nicotinamide adenine dinucleotide (NAD) biosynthesis inhibitor. In some embodiments, the population of particles are polymeric particles. In some embodiments, the polymeric particles comprise poly(lactic-co-glycolic acid) copolymers (PLGA) or poly(lactic acid) and poly(glycolic acid) polymers.

In some embodiments, the load of the NAD biosynthesis inhibitor is between 0.1% to 100% (weight/weight). In some embodiments, the load of the NAD biosynthesis inhibitor is between 1% to 10% (weight/weight). In some embodiments, the load of the NAD biosynthesis inhibitor is between 4% to 5% (weight/weight). In some embodiments, the NAD biosynthesis inhibitor is released from the carrier with a sustained release profile. In some embodiments, the composition does not contain a substantial amount of the NAD biosynthesis inhibitor in crystal form.

In some embodiments, the carrier comprises a population of microparticles. In some embodiments, the mean diameter of the population of microparticles is between 2 μm and 5 μm. In some embodiments, the mean diameter of the population of microparticles is between 3 μm and 4 μm. In some embodiments, the carrier comprises a population of nanoparticles.

In some embodiments, the NAD biosynthesis inhibitor is a nicotinamide phosphoribosyltransferase (NAMPT) inhibitor. In some embodiments, the NAMPT inhibitor is GMX-1778 or FK866. In some embodiments, the composition further comprises a pharmaceutically acceptable excipient.

Other aspects of the present disclosure provide methods of local administration of a metabolic inhibitor, comprising locally administering a therapeutically effective amount of any of the pharmaceutical compositions described herein to a subject in need thereof. In some embodiments, the metabolic inhibitor induces an unacceptable level of toxicity in the subject if administered systemically. In some embodiments, the metabolic inhibitor is a NAD biosynthesis inhibitor.

In some embodiments, the subject has or is suspected of having a cancer. In some embodiments, the cancer is characterized by the presence of one or more mutations in a nucleotide sequence of an isocitrate dehydrogenase 1 gene (IDH1). In some embodiments, the one or more mutations in the nucleotide sequence of IDH1 encode an IDH1 protein variant, wherein the IDH1 protein variant is IDH1 R132H, IDH1 R132C, IDH1 R132G, IDH1 R132S, or IDH1 R132L.

In some embodiments, the cancer is characterized by the presence of one or more mutations in a nucleotide sequence of a histone H3.3 gene (H3F3A). In some embodiments, the one or more mutations in the nucleotide sequence of H3F3A encodes a H3F3A protein variant, wherein the H3F3A protein variant is H3F3A K27M.

In some embodiments, the cancer is characterized by the presence of one or more mutations in a nucleotide sequence of a B-Raf gene (BRAF). In some embodiments, the one or more mutations in the nucleotide sequence of BRAF encodes a B-Raf variant, wherein the B-Raf variant is B-Raf V600E.

In some embodiments, the cancer is a glioma. In some embodiments, the glioma is selected from the group consisting of high grade glioma, diffuse astrocytoma, oligodendroglioma, oligoastrocytoma, secondary glioblastoma, primary glioblastoma, and diffuse intrinsic pontine glioma. In some embodiments, the administering is by intracerebral implantation in the subject. In some embodiments, the subject is undergoing a surgical resectioning procedure. In some embodiments, the pharmaceutical composition is implanted at the tumor margin.

In some embodiments, the method further comprises administering to the subject a therapeutically effective amount of one or more additional cancer therapeutics. In some embodiments, the additional cancer therapeutic is temozolomide. In some embodiments, the additional cancer therapeutic is a NAMPT inhibitor that is administered systemically to the subject.

Other aspects of the present disclosure provide diagnostic methods comprising obtaining a sample from a subject; and detecting one or more mutations in a target nucleic acid in the sample; wherein the detecting is performed by isolating a nucleic acid comprising the target nucleic acid from the sample, and analyzing the nucleic acid for the presence of one or more allele-specific mutations; and wherein the detecting is performed in an intraoperative timeframe. In some embodiments, the intraoperative timeframe is less than 1 hour. In some embodiments, the intraoperative timeframe is less than 35 minutes. In some embodiments, the intraoperative timeframe is less than 30 minutes.

In some embodiments, the subject has or is suspected of having a cancer.

In some embodiments, the subject has or is suspected of having a cancer. In some embodiments, the cancer is characterized by the presence of one or more mutations in a nucleotide sequence of an isocitrate dehydrogenase 1 gene (IDH1). In some embodiments, the one or more mutations in the nucleotide sequence of IDH1 encode an IDH1 protein variant, wherein the IDH1 protein variant is IDH1 R132H, IDH1 R132C, IDH1 R132G, IDH1 R132S, or IDH1 R132L.

In some embodiments, the cancer is characterized by the presence of one or more mutations in a nucleotide sequence of a histone H3.3 gene (H3F3A). In some embodiments, the one or more mutations in the nucleotide sequence of H3F3A encodes a H3F3A protein variant, wherein the H3F3A protein variant is H3F3A K27M.

In some embodiments, the cancer is characterized by the presence of one or more mutations in a nucleotide sequence of a B-Raf gene (BRAF). In some embodiments, the one or more mutations in the nucleotide sequence of BRAF encodes a B-Raf variant, wherein the B-Raf variant is B-Raf V600E.

In some embodiments, the cancer is a glioma. In some embodiments, the glioma is selected from the group consisting of high grade glioma, diffuse astrocytoma, oligodendroglioma, oligoastrocytoma, secondary glioblastoma, primary glioblastoma, and diffuse intrinsic pontine glioma.

In some embodiments, the sample comprises cells, blood, plasma, serum, tissue, and/or cerebrospinal fluid from the subject. In some embodiments, each of the one or more mutations is a single nucleotide polymorphism or a somatic variant in the target nucleic acid.

In some embodiments, the analyzing comprises performing a polymerase chain reaction to detect or amplify the one or more mutation in the target nucleic acid. In some embodiments, the polymerase chain reaction comprises (a) denaturing the nucleic acid isolated from the sample; (b) annealing (i) a forward primer comprising a nucleotide sequence that hybridizes to a first region on a sense strand of the target nucleic acid, (ii) a reverse primer comprising a nucleotide sequence that hybridizes to a second region on an antisense strand of the target nucleic acid, (iii) a probe comprising a nucleotide sequence that is complementary to a mutant allele sequence of the target nucleic acid and located within the region amplified by the forward primer and the reverse primer, and (iv) a peptide nucleic acid (PNA) blocker that hybridizes to a wild-type allele of the target nucleic acid, the PNA blocker comprising a nucleotide sequence that blocks amplification of the wild-type allele, and does not block amplification of the mutant allele, wherein the PNA blocker hybridizes to a region located within the region amplified by the forward and reverse primer; (c) amplifying a DNA amplicon comprising the mutant allele in the first target nucleic acid; (d) detecting the mutant allele in the target nucleic acid; and (e) quantifying the amount of the mutant allele in the target nucleic acid in the sample.

In some embodiments, the probe is a 5′ nuclease probe comprising a cleavable label and quencher. In some embodiments, the cleavable label is a fluorescent moiety. In some embodiments, the probe comprises one or more locked nucleic acid modified nucleotides.

In some embodiments, the target nucleic acid comprises a portion of the nucleotide sequence of an isocitrate dehydrogenase 1 (IDH1) gene. In some embodiments, the mutant allele comprises a variant nucleotide sequence that encodes an IDH1 protein variant, wherein the IDH1 protein variant is IDH1 variant R132H, IDH1 variant R132C, IDH1 variant R132G, IDH1 variant R132S, or IDH1 variant R132L. In some embodiments, the nucleotide sequence of the forward primer is provided by SEQ ID NOs: 2 or 3. In some embodiments, the nucleotide sequence of the reverse primer is provided by SEQ ID NO: 18. In some embodiments, the nucleotide sequence of the probe is provided by any one or more of SEQ ID NOs: 9-13. In some embodiments, the nucleotide sequence of the PNA blocker is provided by SEQ ID NO: 24.

In some embodiments, the target nucleic acid comprises a portion of the nucleotide sequence of a telomerase reverse transcriptase (TERT) gene or the TERT promoter. In some embodiments, the mutant allele of TERT promoter comprises variant TERT promoter, wherein the variant TERT promoter is TERT promoter variant C228T or TERT promoter variant C250T. In some embodiments, the nucleotide sequence of the forward primer is provided SEQ ID NO: 1. In some embodiments, the nucleotide sequence of the reverse primer is provided by SEQ ID NO: 17. In some embodiments, the nucleotide sequence of the probe is provided by any SEQ ID NO: 7 or 8. In some embodiments, the nucleotide sequence of the PNA blocker is provided by SEQ ID NO: 22 or 23.

In some embodiments, the target nucleic acid comprises a portion of the nucleic acid sequence of a nucleotide sequence of a histone H3.3 gene (H3F3A). In some embodiments, a mutant allele of a H3F3A gene encodes a H3F3A protein variant, wherein the H3F3A protein variant is H3F3A variant K27M. In some embodiments, the nucleotide sequence of the forward primer is provided by SEQ ID NO: 4. In some embodiments, the nucleotide sequence of the reverse primer is provided by SEQ ID NO: 19. In some embodiments, the nucleotide sequence of the probe is provided by SEQ ID NO: 14. In some embodiments, the nucleotide sequence of the PNA blocker is provided by SEQ ID NO: 25.

In some embodiments, the target nucleic acid comprises a portion of the nucleic acid sequence of a nucleotide sequence of a B-Raf gene (BRAF). In some embodiments, a mutant allele of a BRAF gene encodes a B-Raf protein variant, wherein the B-Raf protein variant is B-Raf variant V600E. In some embodiments, the nucleotide sequence of the forward primer is provided by SEQ ID NO: 5. In some embodiments, the nucleotide sequence of the reverse primer is provided by SEQ ID NO: 20. In some embodiments, the nucleotide sequence of the probe is provided by SEQ ID NO: 15. In some embodiments, the nucleotide sequence of the PNA blocker is provided by SEQ ID NOs: 26.

In some embodiments, the polymerase chain reaction further comprises a forward control primer and a reverse control primer, wherein the forward control primer and reverse control primer amplify a portion of the nucleic acid sequence of a control gene. In some embodiments, the method further comprises quantifying the amount of the control gene in the nucleic acid of the sample. In some embodiments, the control gene is GADPH.

In some embodiments, the presence of the mutant allele in the sample indicates presence of the cancer in the subject and/or genotype of the cancer in the subject. In some embodiments, the method further comprises selecting a treatment regimen based on the presence of the cancer and/or genotype of the cancer.

Other aspects of the present disclosure provide methods of diagnosis and treatment of a cancer in a subject, comprising performing a surgical procedure on the subject; obtaining a sample from the subject; and determining whether the sample contains a one or more mutations in a target nucleotide sequence in the sample; wherein if the sample is determined to contain the one or more mutations, locally administering an agent with mutation selectivity to the subject during the surgical procedure. In some embodiments, the agent with mutation selectivity is a metabolic inhibitor. In some embodiments, the metabolic inhibitor induces an unacceptable level of toxicity if administered to the subject systemically. In some embodiments, the determining is performed using any of the methods described herein. In some embodiments, the locally administering is performed using any of the methods described herein.

Other aspects of the present disclosure provide methods comprising performing any of the diagnostic methods described herein and performing any of the methods for local administration of an NAD biosynthesis inhibitor described herein. In some embodiments, performing the rapid diagnostic method and performing the method for local administration are both carried out in an intraoperative timeframe. In some embodiments, the intraoperative timeframe is during a surgical resecting procedure.

Also provided herein are nucleic acids comprising any of the nucleotide sequences provided by any one of SEQ ID NOs: 1-26.

Yet other aspects of the present disclosure provide kits for the detection of mutant alleles. In some embodiments, the kit is for the rapid detection of IDH1 variants and comprises (i) a forward primer comprising a nucleotide sequence provided by SEQ ID NOs: 2 or 3; (ii) a reverse primer comprising a nucleotide sequence provided by SEQ ID NOs: 18; (iii) a probe comprising a nucleotide sequence provided by any one of SEQ ID NOs: 9-13, and (iv) a peptide nucleic acid (PNA) blocker comprising a nucleotide sequence provided by SEQ ID NO: 24.

In some embodiments, the kit is for the rapid detection of TERT promoter variants and comprises (i) a forward primer comprising a nucleotide sequence provided by SEQ ID NO: 1; (ii) a reverse primer comprising a nucleotide sequence provided by SEQ ID NO: 17; (iii) a probe comprising a nucleotide sequence provided by SEQ ID NO: 7 or 8, and (iv) a peptide nucleic acid (PNA) blocker comprising a nucleotide sequence provided by SEQ ID NO: 22 or 23.

In some embodiments, the kit is for the rapid detection of H3F3A variants and comprises (i) a forward primer comprising a nucleotide sequence provided by SEQ ID NO: 4; (ii) a reverse primer comprising a nucleotide sequence provided by SEQ ID NO: 19: (iii) a probe comprising a nucleotide sequence provided by SEQ ID NO: 14, and (iv) a peptide nucleic acid (PNA) blocker comprising a nucleotide sequence provided by SEQ ID NO: 25.

In some embodiments, the kit is for the rapid detection of BRAF variants and comprises (i) a forward primer comprising a nucleotide sequence provided by SEQ ID NO: 5; (ii) a reverse primer comprising a nucleotide sequence provided by SEQ ID NO: 20: (iii) a probe comprising a nucleotide sequence provided by SEQ ID NO: 15; and (iv) a peptide nucleic acid (PNA) blocker comprising a nucleotide sequence provided by SEQ ID NO: 26.

In some embodiments, the kit further comprises instructions to locally administer a therapeutic agent to a subject if a mutant allele is detected. In some embodiments, the therapeutic agent is any of the pharmaceutical compositions described herein. In some embodiments, the kit further comprises any of the pharmaceutical composition described herein. In some embodiments, the kit further comprises one or more additional components selected from the group consisting of a DNA polymerase, deoxynucleotide triphosphates, and a buffer.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the Figures. The invention is capable of other embodiments and of being practiced or of being carried out in various ways. Also, the phraseology and terminology used herein is for the purpose of description and should not be regarded as limiting.

### BRIEF DESCRIPTION OF THE DRAWINGS

The drawings are first described. It is to be understood that the drawings are exemplary and not required for enablement of the invention.

FIGS. 1A-ID show that IDH1-mutated diffuse astrocytomas demonstrate local disease progression. FIG. 1A shows a series of MRIs from a representative patient with diffuse astrocytoma. As indicated by the arrowheads, disease progression is shown adjacent to the initial lesion. FIG. 1B shows representative micrographs assessing the tumor burden by IDH1 R132H immunohistochemistry. The progressive disease burden can be seen with focal nodular tumor cells. FIG. 1C shows a graphic representation of retrospective analysis of 130 patients with IDH-mutated diffuse astrocytomas. Local disease progression within 2 cm of the initial lesion was observed in 81.8% of patients with median progression free survival of 4.7 years. FIG. 1D shows a schematic of a method for delivering genotype-directed local therapy during neurosurgical resection to target therapy against remaining cancer cells at the surgical margin.

FIGS. 2A-2D show that systemic administration of NAMPT inhibitors results in toxicity. FIG. 2A shows maximum drug concentrations obtained after SCID mice of 6-7 weeks old were administered the brain permeable NAMPT inhibitor, GMX-1778 (250 mg/kg, oral gavage). GMX-1778 concentrations were assessed in plasma and in the brain at 30 minutes, 2 hours, 6 hours, and 24 hours (n=7 mice for each time point). Maximum drug concentrations were noted at 2 hours post-administration (18.0±3.6 μM in plasma and 3.0±1.5 μM in brain). FIG. 2B shows administration of GMX-1778 (250 mg/kg) in adult SCID mice resulted in weight loss within 6 days following administration (17.9±1.1 g, n=5 vs 21.8±0.6 g, n=9 control dextrose treated animals, p<0.05). FIG. 2C shows a diagram of a chemistry panel indicating that GMX-1778-treated animals had anemia (hemoglobin 6.7±0.8 g/dL, n=4 vs 9.2±0.5 g/dL, n=5, p<0.05) and uremia (20.5±1.9 mg/dL, n=4, vs 15±0.5 mg/dL, n=4, p<0.05). Bicarbonate level was not tested (NT) in the chemistry panel. FIG. 2D shows representative micrographs of liver sections from mice treated with GMX-1778 and control mice that did not receive GMX-1778. Arrowheads indicate signs of eosinophilic cholangitis. Scale bar 40 μm, inset magnified 2× further.

FIGS. 3A-3H show that example microparticle formulation for sustained release of NAMPT inhibitors demonstrate selective in vitro activity against IDH-mutated cells. FIG. 3A shows a table of example microparticle formulations containing NAMPT inhibitors Microparticles were loaded with GMX-1778 using a single emulsion technique with modification of the formulation parameters, resulting in varying levels of drug loading as measured by HPLC. Scanning electron microscopy revealed FIG. 3B presents a representative scanning electron micrograph showing drug crystals in the formulation with highest drug load (Formulation A). FIG. 3C a representative scanning electron micrograph showing no drug crystals in the formulation with lowest drug load (Formulation H). FIG. 3D shows a representative scanning electron micrograph showing a pure microparticle composition in a formulation with 4.5% drug load (Formulation I). FIG. 3E presents a plot showing the mean particle size is 3.4+/−1.6 um as measured by SEM imaging followed by processing by ImageJ software. FIG. 3F shows viability of MGG152 cells, an IDH1 R132H variant human glioma cell line, following treatment with microparticles containing a total of 1 μM GMX-1778 or 1 μM FK-866. A time-dependent decrease in cell viability was observed in cells treated with microparticles containing GMX-1778 or FK-866 compared to cells treated with similarly prepared inhibitor-free microparticles over 72 hours (96.3±0.2% for GMX-1778, n=3, and 80.5±3.4% for FK-866, n=3). HPLC analysis of GMX-1778 in the media of cells treated with the sustained release microparticle formulation revealed concentrations of 40.4±2.3 nM at 24 hours and 63.8±3.7 nM at 72 hours (as shown in FIG. 14). FIG. 3G shows viability of the indicated cell lines following treatment with the GMX-1778 microparticle formulation. Time-dependent activity of the GMX-1778 microparticle formulation was noted in the MGG152 cell line and in an additional IDH1 R132C variant chondrosarcoma cell line, HT1080 (n=3), but not the IDH wildtype glioblastoma cell line, U87 (n=3). FIG. 3H presents normalized viability of cell lines following treatment with the GMX-1778 microparticle formulation 48 hours following treatment. In the plot, for each cell line tested, the left column refers to FK866 and the right column refers to GMX-1778.

FIGS. 4A-4B show the rapid methods described herein allow for detection and the categorization of recurrent mutations in gliomas, for use of targeted local therapy. FIG. 4A shows a schematic of the rapid genotyping assay. Briefly, the assay utilizes TaqMan-based probes for fluorescence-based detection of mutant alleles with clamping of the wildtype allele amplicon via peptide nucleic acid (PNA) oligonucleotides. The assay allows for detection of tumor variants to an allelic fraction of 1% within 30 minutes. FIG. 4B presents validation of genotyping assay. The mutation call for IDH1 R132, TERT promoter variants, H3F3A K27M and BRAF V600E was correlated in 87 brain tumor specimens from a clinically annotated database.

FIGS. 5A-5C show intratumoral implantation of sustained release microparticle formulations demonstrate in vivo activity and result in increased survival in an IDH1 variant orthotopic model. FIG. 5A shows representative scans of SCID mice that were intracerebrally implanted with U87 or MGG152 cells expressing luciferase (2×10⁵ cells). Bioluminescence imaging was performed 15 days following implantation to establish a baseline. Microparticles containing GMX-1778 to reach a concentration of 5 μM in a sphere with radius 2 mm were implanted at that time point. Bioluminiscence imaging was performed on a weekly basis. No significant difference in survival was noted in animals that were implanted with the IDH wildtype U87 glioblastoma cells and treated with the GMX-1778 microparticle formulations (n=4 for blank and n=5 for GMX-1778 microparticles). FIG. 5B shows that GMX-1778 microparticles significantly decreased tumor growth compared to control tumor cells that did not receive the microparticles. FIG. 5C shows increased survival of mice implanted with MGG152 cells. Mice that received the microparticle formulations survived longer than the control mice in the orthotopic MGG152 model (58 days, n=5 for blank, vs 75.5 days, n=6 for GMX-1778 microparticles, p<0.05).

FIG. 6 shows a retrospective analysis of 130 patients with IDH1 variant diffuse astrocytomas. The analysis revealed local progression within 2 cm of the initial lesion in 81.8% of patients with median progression free survival of 4.7 years. Distal failure was noted in 18.2% of patients in this cohort with a median progression free survival of 5.1 years.

FIG. 7 shows MGG152 cells treated with PLGA microparticle formulations with GMX1778 (0.031±0.001 of control at 72 hours, n=3) or PLGA microparticle formulations with FK866 (0.618±0.078 of control at 72 hours, n=3). A time-dependent decrease in NAD+ levels was observed when normalized to cells treated with microparticles without drug.

FIG. 8 shows sequencing analysis validating the wildtype and mutant control genomic extracts. Wildtype genomic extracts were obtained from HEK293T cells. Mutant (variant) genomic templates were obtained from MGG152 (IDH1 variant R132H), HT1080 (IDH1 variant R132C), U87 (TERT variant C228T), (Hs683 (TERT variant C250T), DIPG8 (H3F3A variant K27M), and HTB-38 (BRAF variant V600E) cells. Input extracts were amplified using primers listed in Table 1 for the respective templates, and amplicon was sequenced by Sanger sequencing. Top to bottom and left to right the sequences in this figure correspond to SEQ ID NOs: 27-33.

FIGS. 9A-9D present representative DNA gels showing optimization of PNA oligonucleotide clamp (blocking) of wildtype allele amplification. FIG. 9A shows that PNA blocking oligonucleotides clamp wildtype alleles (TERT C228 (top gel) or wildtype TERT C250 (bottom gel)), but allow for amplification of mutant template (denoted by asterisk) at annealing and extension temperature of 63.5° C. FIG. 9B shows that a PNA blocking oligonucleotide for wildtype H3F3A K27 was optimized at 250 nM PNA for 63.5° C. FIG. 9C shows that a PNA blocking oligonucleotide for wildtype IDH1 R132 was optimized at 500 nM for 63.5° C. FIG. 9D shows that a PNA blocking oligonucleotide for wildtype BRAF V600 was optimized at 250 nM for 63.5° C.

FIGS. 10A-10F show the sensitivity of the genotyping assay. Within the 27 minute assay timeframe, mutant alleles diluted in wildtype genomic extract could still be specifically detected at an allele fraction of 1%. FIG. 10A shows detection of IDH1 variant R132H. FIG. 10B shows detection of IDH1 variant R132C. FIG. 10C shows detection of TERT variant C228T. FIG. 10D shows detection of TERT variant C250T. FIG. 10E shows detection of H3F3A variant K27M. FIG. 10F shows detection of BRAF variant V600E. Genomic extracts from each of the mutant cell types were serially diluted with wildtype genomic extract from HEK293T. The input DNA concentration for reactions was 10 ng as quantified using a PicoGreen assay.

FIGS. 11A-11B show the optimization of the rapid genotyping assay with an internal GAPDH control. The rapid genotyping assay was multiplexed to detect GAPDH (indicated with an arrow) to ensure assay integrity. Using the assay parameters described herein, the mutants were detected at 10% allelic fraction (right column) for each of IDH1 variants R132(H, C, S, L, G), TERT variants C228T and C250T, H3F3A variant K27M and BRAF variant V600E. Performing the assay using wildtype DNA only resulted in detectable signal for the GAPDH control (middle column).

FIGS. 12A-12B show plots and representative micrographs validating the luciferase- and mCherry-expressing human glioma cells. MGG152 and U87 were infected with lentiviruses containing the luciferase and mCherry construct. Cells were visualized by mCherry expression (inset) and sorted by fluorescence to select cells expressing the construct. Subsequently, luciferase activity was measured as luminescence following addition of d-luciferin substrate. Notably, while both cells demonstrated a linear relationship between luciferase activity and number of cells, the overall intensity of luminescence generated by MGG152 was 70-fold less compared to U87. FIG. 12A shows MGG152 cells. FIG. 12B shows U87 cells.

FIGS. 13A-13B show that tumor volume and survival of U87 tumor cells were not affected by local implantation of NAMPT inhibitor in an orthotopic human GBM murine model. Fifteen days following intracranial implantation of 2×10̂5 U87-Luc cells, PLGA microparticle formulations with GMX1778 or empty microparticles were injected into the tumor. FIG. 13A shows U87 tumor growth rate was not significantly affected by the GMX-1778 microparticles. FIG. 13B shows survival of mice implanted with the U87 tumor cells was not affect by the GMX-1778 microparticles in this U87 orthotopic model (29.5 days for blank, n=4, vs 29 days for GMX-1778, n=5).

FIG. 14 presents a graph showing release of GMX-1778 from microparticles at the indicated times. Cells were treated with microparticles for 1, 2 and 3 days. At these times, cells and microparticles were separated by centrifugation, and the media was collected. Drug (GMX-1778, “GMX”) was extracted from the media by liquid-liquid extraction using ethyl acetate for 24 h. The ethyl acetate layer was collected and evaporated to dryness under vacuum. The dried residue was dissolved in methanol and drug concentration was analyzed using HPLC. Data is represented as mean±S.D., n=3

### DETAILED DESCRIPTION

According to the revised WHO 2016 neuropathology diagnostic criteria, greater than 90% of adult diffuse gliomas are characterized by recurrent hotspot mutations in the metabolic enzyme isocitrate dehydrogenase 1 (IDH1) or its homolog isocitrate dehydrogenase 2 (IDH2), the telomerase reverse transcriptase (TERT) promoter, histone H3.3 (H3F3A), and signaling kinase BRAF. See, e.g., Louis et al. Acta Neuropathol. (2016) 131: 803-820; Yan et al. N. Engl. J. Med. (2009) 360: 765-773; Caner Genome Atlas Research Network et al. N. Engl. J. Med. (2015) 372: 2481-2498; Schwartzentruber, et al. Nature (2012) 482: 226-231; Dahiya et al. Neuro-Oncol. (2014) 16: 318-319.

Described herein are methods for rapidly genotyping specific genetic loci in samples, thereby accelerating the acquisition of diagnostic information into the intraoperative timeframe. IDH1 mutant gliomas represent example cancers for the development of a genotype-based local therapies. Sustained local control through neurosurgical resectioning has been central to treating gliomas and prolonging survival in these patients; nevertheless, recurrence arising from the tumor-infiltrated surgical margin is common. Effective therapeutic agents such as NAMPT inhibitors have thus far been infeasible for systemic administration due to systemic toxicities in the subject. Provided herein are rapid genotyping methods for intraoperative diagnosis, and the pharmaceutical compositions for the local administration of therapeutic agents (e.g., metabolic inhibitors, such as NAMPT inhibitors). Accordingly, the methods described herein allow for genotype-directed application of a precision intraoperative local therapy for cancers, such as IDH1-mutant gliomas. Also provided herein are kits comprising components, such as primers and probes to specifically identify and genotype cancer, for use in the rapid diagnostic methods described herein.

The phraseology and terminology used herein is for the purpose of description and should not be regarded as limiting. The use of “including.” “comprising,” or “having,” “containing,” “involving,” and variations thereof herein, is meant to encompass the items listed thereafter and equivalents thereof as well as additional items.

### Methods of Local Administration

Aspects of the present disclosure provide methods of locally administering therapeutically effective amounts of a therapeutic agent to a subject. In some embodiments, the therapeutic agent is administered based on identification of a mutant genotype in cells of a sample obtained from the subject. In some embodiments, the mutant genotype is identified using any of the diagnostic methods described herein. In some embodiments, the methods involve administering a therapeutically effective amount of an agent with mutation selectivity. In some embodiments, the methods involve administering a therapeutically effective amount of a metabolic inhibitor, such as a nicotinamide adenine dinucleotide (NAD) biosynthesis inhibitor.

In general, systemic administration of some therapeutic agents may result in a level of toxicity to the subject, the severity of which may prevent its systemic therapeutic use. However, in some embodiments, local administration of such agents may result in fewer or no toxic effects. As used herein, the term “toxic” refers to any detrimental, deleterious, harmful, or otherwise negative side effect on the subject, tissue, or cell during or after administration or contact of a subject, tissue, or cells with the agent. In some embodiments, systemic administration of a metabolic inhibitor results in toxicity in the subject. Examples of toxic effects resulting from systemic administration of the agent include, without limitation, hematologic toxicity, retinal toxicity, renal toxicity, hepatotoxicity, and adverse immunologic responses. For example, it is appreciated in the art that systemic administration of inhibitors of nicotinamide phosphorylribosyltranferase (NAMPT), as described herein, result in toxicity in subjects, such as hematologic and retinal toxicities. See, e.g., Zabka et al. Toxicol. Sci. (2015) 144(1): 163-172.

As will be evident to one of ordinary skill in the art, therapeutic agents generally result in a beneficial result (e.g, therapeutic result) when administered to a subject. In additional, therapeutic agents may also have one or more toxic effects or a level of toxicity when administered to the subject. When evaluating a candidate agent, one of the factors generally considered is the ratio the beneficial result conferred by the agent to the level of toxicity of the agent. The level of toxicity of the agent may depend, for example, on the route of administration of the agent, the dosage of the agent administered, the frequency of dosage, as well as any subject-specific factors, such as the age, gender, and immunological status of the subject, and whether any additional therapeutics have also been administered to the subject. An agent for which the level of toxicity significantly outweighs the beneficial results conferred, is considered to have an unacceptable level of toxicity and may not be used for administration to subjects (e.g., at a specific dosage, by a particular administration route, or a particular subset of subjects). In some embodiments, as described herein, an agent is locally administered to a subject, wherein if the same agent was systemically administered to the subject, the agent would be considered to have an unacceptable level of toxicity.

In some embodiments, the toxicities associated with systemic administration of a therapeutic agent, such as a NAMPT inhibitor, may be overcome by locally administering the therapeutic agent. As used herein, the term “locally administer,” “local administration,” and “local delivery” refer to a targeted administration of the therapeutic agent to a specific location or region (or a plurality of locations or regions) of a subject while applying appreciably less, optionally no, agent to one or more other specific locations or regions of the subject. In some embodiments, the locally administered therapeutic agent is administered to a target site, e.g., a specific portion of the body for which activity of the agent is desired (e.g., the site of cancerous cells). Local administration of an agent may allow for use of a lower dosage of the agent, avoid toxicities encountered by other modes of administration (e.g., systemic), and/or avoid interaction with other systemically administered medications.

In contrast to systemic administration which may result in dispersal of the therapeutic agent throughout the body (or substantially throughout the body) of the subject, local administration of the therapeutic agent does not substantially disperse throughout the body. In some embodiments, a locally administered agent is retained at the target site of administration.

Local delivery of an agent generally allows for a higher concentration of the agent at the target site of administration, for example as compared to systemic concentrations of the agent or concentrations of the agent at one or more distal sites. This may result in a reduction in toxicities associated with the agent and/or improved efficacy (e.g., due to higher local concentration of the drug and/or reduced degradation of the agent during transit to the target site).

In general, a locally administered therapeutic agent is not substantially detected systemically in the subject or at a site distal to the target site. In some embodiments, the activity of a therapeutic agent that is a locally administered agent is not substantially detected systemically in the subject or at a site distal to the target site.

Local administration may be performed by any method known in the art. For example, a locally administered therapeutic agent (e.g., an agent with mutation selectivity, a metabolic inhibitor, such as a NAMPT inhibitor) may be injected, infused, or implanted to a target site. The therapeutic agent may be locally administered in any form known in the art, such as a liquid, a particle, or a wafer. Local delivery may be direct to the target site or nearly direct to a site that is sufficiently proximal to the target site such that the therapeutic agent exhibits the desired activity at the target site. In some embodiments, the local administration is to the brain, for example by intracranial or intracerebral administration (e.g., intracranial or intracerebral injection, infusion or implantation). In some embodiments, the local administration is to the cerebrospinal fluid, for example by intrathecal administration. Administration routes for local administration may include oral, parenteral, intramuscular, intranasal, sublingual, intratracheal, inhalation, intracranial, ocular, vaginal, and rectal routes. Selection of an appropriate route of local administration will be evident to one of ordinary skill in the art and may depend on factors such as the target site to which administration of the therapeutic agent is desired.

In some embodiments, the therapeutic agent is administered locally to a subject while the subject is undergoing a surgical procedure, such as surgical resectioning. As used herein, the term “surgical resectioning” refers to a surgical procedure during which a portion of an organ or tissue is removed from the subject. Surgical resectioning is a therapeutic approach that is frequently used in the treatment of cancer to remove cancer cells (e.g., a tumor) from a subject having cancer. In some embodiments, the portion of the organ or tissue that is removed contains cancer cells or cells suspected of being cancer cells. In some embodiments, the portion of the organ or tissue that is removed is a tumor, a portion thereof, or is suspected being a tumor.

In some embodiments, a therapeutic agent (e.g., a NAMPT inhibitor) is locally administered to a target site while the subject is undergoing a surgical procedure, such as surgical resectioning. Administration of a therapeutic agent to during a surgical procedure may allow direct access to a target site that may be otherwise difficult to access, such as a privileged site or across the blood-brain barrier.

During surgical resectioning for cancer treatment, maximal removal of tumor cells is desired. Tumor cells remaining at the site may have the capacity to replicate, leading to recurrence of the cancer and potentially spread to additional sites in the subject. However, pursuing larger marginal resection is difficult due to adjacent healthy (non-cancerous) tissues. In the case of brain tumors, the adjacent healthy tissues may be neurologically-normal, functional brain tissue. Therefore, expansion of the area of resectioning to include these margins may lead to unwanted late-toxicity to the surrounding normal parenchyma.

A therapeutic agent may be locally administered the site of resectioning to reduce the growth, replication, and/or spread of any remaining tumor cells at or near the site of resectioning. In some embodiments, local administration of a therapeutic agent to the site of resectioning (e.g., the tumor margin) reduces the number of remaining tumor cells and/or reduces or delays cancer recurrence. In some embodiments, a therapeutic agent is locally administered to the site of surgical resectioning following removal of a tumor (or a portion thereof). In some embodiments, a therapeutic agent is locally administered to the tumor margin following removal of a tumor (or a portion thereof). The term “tumor margin” is used herein to refer to a region between tumor cells and healthy (non-cancerous) cells. In the case of brain tumors, the tumor margin refers to the region between the brain tumor cells and healthy brain tissue.

As described herein, a therapeutic agent may be locally administered to a target site by injecting, infusing, and/or implanting the agent into the site. The target site for local administration will generally depend on the type of cancer and/or the location of the cancer to be treated. In some embodiments, the subject has a cancer, such as a brain cancer. In some embodiments, the target site to which administration of the agent is desired is the brain. In some embodiments, the subject is undergoing a surgical resectioning to remove a brain tumor, such as a glioma.

Agents with Mutation Selectivity

Aspects of the present disclosure provide pharmaceutical compositions and methods of administering agents with mutation selectivity to a subject. As used herein, the term “agent with mutation selectivity” refers to an agent that has selective toxicity or increased toxicity towards cells (e.g., cancer cells) having one or more specific mutation as compared to cells that do not have the mutation. For example, IDH1 mutant cancers are sensitive to NAD+ depletion, therefore administration of an agent that reduces NAD+ levels may be cytotoxic to IDH1 mutant cancers. See, e.g., Tateishi et al. Cancer Cell (2015) 28(6): 773-784.

In some embodiments, the methods involve performing a surgical procedure on the subject, obtaining a sample from the subject; and determining whether the sample contains one or more mutations in a target nucleotide sequence in the sample, and if the sample is determined to contain the one or more mutations, locally administering an agent with mutation selectivity to the subject during the surgical procedure.

In some embodiments, the agent with mutation selectivity is a metabolic inhibitor. In some embodiments, the agent with mutation selectivity has toxicity toward cells having one or more mutations in IDH1 (an IDH1 variant). In some embodiments, the agent with mutation selectivity has toxicity toward cells having one or more mutations in the TERT promoter (a TERT promoter variant). In some embodiments, the agent with mutation selectivity has toxicity toward cells having one or more mutations in H3F3A (a H3F3A variant). In some embodiments, the agent with mutation selectivity has toxicity toward cells having one or more mutations in BR4F (a BRAF variant).

### Metabolic Inhibitors

Aspects of the present disclosure provide pharmaceutical compositions and methods of administering therapeutic agents to a subject. In some embodiments, the therapeutic agents are metabolic inhibitors. In some embodiments, metabolic inhibitors are locally administered to the subject if a genotype is detected in a sample from the subject, for example using the diagnostic methods described herein.

As used herein, the term “metabolic inhibitor” refers to any agent that inhibits a metabolic pathway in a cell. Metabolic inhibitors may be in any form known in the art, such as nucleic acid inhibitors and small molecule inhibitors. In some embodiments, the metabolic inhibitor reduces expression of an enzyme involved in the metabolic pathway. In some embodiments, the metabolic inhibitor reduces the activity of an enzyme and/or cofactor involved in the metabolic pathway. In some embodiments, inhibition of a metabolic pathway reduces the production of a product, e.g., a metabolite. In some embodiments, inhibition of a metabolic pathway depletes the level of a product in the cell, e.g., a metabolite.

The level of activity of metabolic pathways (e.g., the NAD biosynthesis pathway) may be assessed by conventional methods known in the art, for example by directly or indirectly quantifying the amount of a metabolite in a cell or population of cells.

In some embodiments, the level of the metabolite in a cell following local administration of the metabolic inhibitor (e.g., an inhibitor of NAD biosynthesis, such as a NAMPT inhibitor) is reduced by at least 5%, 10%, 15%, 20%, 25%, 30%, 35%, 40%, 45%, 50%, 55%, 60%, 65%, 70%, 75%, 80%, 85%, 90%, 95% or more as compared to the level of the metabolite in a cell that has not been locally administered the metabolic inhibitor. In some embodiments, the level of the metabolite in a cell following local administration of the metabolic inhibitor (e.g., an inhibitor of NAD biosynthesis, such as a NAMPT inhibitor) is reduced by at least 5%, 10%, 150%, 20%, 25%, 30%, 35%, 40%, 45%, 50%, 55%, 60%, 65%, 70%, 75%, 80%, 85%, 90%, 95% or more as compared to the level of the metabolite in a cell has not been treated with the metabolic inhibitor. In some embodiments, the level of the metabolite in a cell following local administration of the metabolic inhibitor (e.g., an inhibitor of NAD biosynthesis, such as a NAMPT inhibitor) is reduced by at least 5%, 10%, 15%, 20%, 25%, 30%, 35%, 40%, 45%, 50%, 55%, 60%, 65%, 70%, 75%, 80%, 85%, 90%, 95% or more as compared to the level of the metabolite in a cell in a subject that has not received local administration of the metabolic inhibitor.

In some embodiments, a metabolic inhibitor is locally administered to a subject to reduce or deplete cellular levels of nicotinamide adenine dinucleotide (NAD). The terms “NAD” and “NAD+” are used interchangeably throughout. NAD can be synthesized through two cellular pathways: a de novo pathway and a salvage pathway. In general, the de novo pathway generates NAD from tryptophan, whereas the salvage pathway generates NAD from nicotinamide, nicotinic acid, and nicotinamide riboside. See, e.g., Belenky et al. Trends Biochem. Sci. (2007) 32(1): 12-19. In the salvage pathway, nicotinamide may be converted to NAD+ by two enzymes: nicotinamide phosphoribosyltransferase (NAMPT) and nicotinamide mononucleotide adenyltransferase. NAMPT (EC 2.4.2.12) is the rate-limiting enzyme of the salvage pathway and catalyzes the transfer of a phosphoribosyl group from 5-phosphoribosyl-1-pyrophosphate (PRPP) to nicotinamide, forming nicotinamide mononucleotide (NMN), a key NAD pathway intermediate.

In some embodiments, the metabolic inhibitor is an inhibitor of NAD biosynthesis, such as an inhibitor of an enzyme or cofactor involved in the de novo NAD biosynthetic pathway or an inhibitor of an enzyme or cofactor involved in the salvage NAD biosynthetic pathway. In some embodiments, the inhibitor of NAD biosynthesis is an NAMPT inhibitor.

As used herein, the terms “NAMPT inhibitor” and “nicotinamide phosphoribosyl transferase inhibitor” refer to an inhibitor that reduces the activity of NAMPT. The term “NAMPT inhibitor” also includes a pharmaceutically acceptable salt of a NAMPT inhibitor and/or a prodrug of a NAMPT inhibitor. In some embodiments, the activity of NAMPT in a cell following treatment with a NAMPT inhibitor is reduced by at least 5%, 10%, 15%, 20%, 25%, 30%, 35%, 40%, 45%, 50%, 55%, 60%, 65%, 70%, 75%, 80%, 85%, 90%, 95% or more, as compared to the level of NAMPT activity prior to treatment with the NAMPT inhibitor. In some embodiments, the activity of NAMPT in a cell following treatment with a NAMPT inhibitor is reduced by at least 5%, 10%, 15%, 20%, 25%, 30%, 35%, 40%, 45%, 50%, 55%, 60/o, 650%, 70%0, 75%, 80%, 85%, 90%, 95%, 96%, 97%0, 98%, 99% or more, as compared to the level of NAMPT activity in a control cell, e.g., a cell that was not treated with the NAMPT inhibitor. Examples of NAMPT inhibitors include, without limitation, FK866 (also referred to as APO866), GPP 78 hydrochloride, ST 118804, STF31, pyridyl cyanoguanidine (also referred to as CH-828), GMX-1778, and P7C3. Additional NAMPT inhibitors are known in the art and may be suitable for use in the compositions and methods described herein. See, e.g., PCT Publication WO 2015/054060, U.S. Pat. Nos. 8,211,912, and 9,676,721, which are incorporated by reference herein in their entireties. In some embodiments, the NAMPT inhibitor is FK866. In some embodiments, the NAMPT inhibitor is GMX-1778.

In some embodiments, the level of NAD in a cell following local administration of the inhibitor of the NAD biosynthesis pathway (e.g., NAMPT inhibitor) is reduced by at least 5%, 10%, 15%, 20%, 25%, 300, 35%, 40, 40%, 45%, 50%, 55%, 60%, 65%, 70%, 75%, 80%, 85%, 90%, 95%, 96%, 97%, 98%, 99% or more as compared to the level of NAD in a cell in a subject that has not received local administration of the NAD biosynthesis pathway inhibitor. In some embodiments, the activity of NAMPT in a cell following local administration of the NAMPT inhibitor is reduced by at least 5%^{,} 10%, 15%, 20%, 25%, 30%, 35%, 400%, 45%, 50%, 55%, 60%, 65%, 70%, 75%, 80%, 85%, 90%, 95% or more as compared to the level of NAMPT activity in a cell in a subject that has not received local administration of the NAMPT inhibitor. Methods for assessing the levels of NAD or activity of NAMPT in a cell will be evident to one of ordinary skill in the art.

In some embodiments, local administration of a metabolic inhibitor (e.g., an inhibitor of the NAD biosynthesis pathway, such as a NAMPT inhibitor) reduces the growth and/or proliferation of cancer cells, such as cancer cells within proximity to the site of local administration of the metabolic inhibitor. In some embodiments, local administration of a metabolic inhibitor (e.g., an inhibitor of the NAD biosynthesis pathway, such as a NAMPT inhibitor) is cytotoxic to cancer cells, and kills cancer cells within proximity to the site of local administration of the metabolic inhibitor. In some embodiments, local administration of a NAMPT to a target site, such as a tumor margin, increases the survival rate of the subject.

In some embodiments, the growth and/or proliferation of cancer cells in a subject is reduced by at least 5%, 10%, 15%, 20%, 25%, 30%, 35%, 40%, 45%, 500, 55%, 60%, 65%, 70%, 75%, 80%, 85%, 90%, 95%, 96%, 97%, 98%, 99% or more following local administration of a therapeutic agent (e.g., a NAD biosynthesis inhibitor, such as a NAMPT inhibitor) as compared to the level of NAD in a cell in a subject that has not received local administration of the therapeutic agent. In some embodiments, the growth and/or proliferation of cancer cells in a tumor margin of a subject is reduced by at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or more following local administration of a therapeutic agent (e.g., a NAD biosynthesis inhibitor, such as a NAMPT inhibitor) to the tumor margin as compared to the growth and/or proliferation of cancer cells in a subject that has not received local administration of the therapeutic agent.

In some embodiments, the number of cancer cells (e.g., cancer burden) in a subject is reduced by at least 5%, 10%, 15%, 20%, 25%, 30%, 35%, 40%, 450%, 50%, 55%, 60%, 65%, 70%, 75%, 80%, 85%, 90%, 95%, 96%, 97%, 98%, 99% or more following local administration of a therapeutic agent (e.g., a NAD biosynthesis inhibitor, such as a NAMPT inhibitor) as compared to the number of cancer cells in a subject that has not received local administration of the therapeutic agent. In some embodiments, the number of cancer cells (e.g., cancer burden) in a subject is reduced by at least 5%, 100%, 15%, 20%, 25%, 30%, 35%, 40%, 45%, 50%, 55%, 60%, 65%, 70%, 75%, 80%, 85%, 90%, 95%, 96%, 97%, 98%, 99% or more following local administration of a therapeutic agent (e.g., a NAD biosynthesis inhibitor, such as a NAMPT inhibitor) as compared to the number of cancer cells in the same subject prior to local administration of the therapeutic agent.

In some embodiments, the number of cancer cells in a tumor margin of a subject is reduced by at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or more following local administration of a therapeutic agent (e.g., a NAD biosynthesis inhibitor, such as a NAMPT inhibitor) to the tumor margin as compared to the number of cancer cells in a subject that has not received local administration of the therapeutic agent. In some embodiments, the number of cancer cells in a tumor margin of a subject is reduced by at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or more following local administration of a therapeutic agent (e.g., a NAD biosynthesis inhibitor, such as a NAMPT inhibitor) to the tumor margin as compared to the number of cancer cells in the same subject prior to local administration of the therapeutic agent.

In some embodiments, local administration of a therapeutic agent (e.g., a NAD biosynthesis inhibitor, such as a NAMPT inhibitor) results in a reduction in the rate or incidence of cancer reoccurrence as compared to the rate or incidence of cancer reoccurrence in subjects that have not received local administration of the therapeutic agent. In some embodiments, the rate or incidence of cancer reoccurrence is reduced by at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or more following local administration of a therapeutic agent (e.g., a NAD biosynthesis inhibitor, such as a NAMPT inhibitor) as compared to the rate or incidence of cancer reoccurrence in subjects that have not received local administration of the therapeutic agent.

### Treatment of Cancer

The pharmaceutical compositions and methods described herein may be used for the rapid diagnosis and/or treatment of cancer in a subject. The terms “patient,” “subject,” or “individual” may be used interchangeably and refer to a subject treated by or subjected to the methods described herein. In some embodiments, the subject is a human or non-human animal. In some embodiments, a subject has or is suspected of having a cancer. In some embodiments, the subject is undergoing a surgical procedure to remove a tumor or portion thereof. In some embodiments, the subject is undergoing a surgical resectioning procedure for the treatment of cancer.

In some embodiments, the pharmaceutical compositions described herein are locally administered to a subject based on the results of the diagnostic methods described herein. For example, if a sample obtained from the subject is determined to have a cancer with a mutant genotype, any of the pharmaceutical compositions described herein may be locally administered to the subject to treat the cancer. In some embodiments, the subject is undergoing a surgical resectioning procedure to treat the cancer and the pharmaceutical compositions described herein are locally administered to the subject to increase the efficacy of the cancer treatment.

According to embodiments that involve administering a therapeutically effective amount of the compositions as provided herein, “therapeutically effective” or “an amount effective to treat” denotes the amount of the a therapeutic agent (e.g., a metabolic inhibitor) or composition thereof needed to inhibit or reverse a disease condition. Determining a therapeutically effective amount specifically depends on such factors as toxicity and efficacy of the medicament. These factors will differ depending on other factors such as potency, relative bioavailability, patient body weight, severity of adverse side-effects, and mode of administration. Toxicity may be determined using methods well known in the art. Efficacy may be determined utilizing the same guidance. Efficacy, for example, can be measured by a decrease in the progress of the cancer. A pharmaceutically effective amount, therefore, is an amount that is deemed by the clinician to be toxicologically tolerable, yet efficacious.

As also used herein, the term “treating” refers to the application or administration of a composition including one or more active agents to a subject, who has a target disease (e.g., cancer), or a predisposition or risk of having the disease (e.g., cancer), with the purpose to cure, heal, alleviate, relieve, alter, remedy, ameliorate, improve, or affect the disease, a symptom of the disease, or the predisposition or risk toward the disease or recurrence of the disease. In some embodiments, the subject has or is at risk of having cancer.

In some embodiments, the compositions and methods described herein are for the diagnosis and/or treatment of cancer. The compositions and methods described herein for the local administration of a therapeutic agent (e.g., a metabolic inhibitor) may be used to increase the efficacy of cancer treatment. The compositions and methods described herein for the local administration of a therapeutic agent (e.g., metabolic inhibitor) may be used to increase the efficacy of a surgical resectioning procedure to remove cancer cells. In some embodiments, local administration of a therapeutic agent (e.g., a metabolic inhibitor) reduces the number of cancer cells remaining at a site following surgical resectioning. In some embodiments, local administration of a therapeutic agent (e.g., a metabolic inhibitor) reduces the incidence or risk of cancer metastasis. In some embodiments, local administration of a therapeutic agent (e.g., a metabolic inhibitor) reduces the incidence or risk of cancer recurrence.

Examples of cancers include, without limitation glioma, glioblastoma multiforme, melanoma, small cell lung cancer, non-small cell lung cancer, bone cancer, breast cancer, ovarian cancer, melanoma, uveal melanoma, bladder cancer, colon cancer, and lung cancer. In some embodiments, the cancer is cholangiocarinoma. In some embodiments, the cancer is a cartilaginous tumor.

In some embodiments, the cancer is located in a privileged site in the subject, such as beyond the blood-brain barrier. In some embodiments, the cancer is a cancer of the central nervous system or brain. Examples of cancers of the brain and/or central nervous system include, without limitation, acoustic neuroma, astrocytoma (e.g., Grade I—pilocytic astrocytoma; Grade II—low-grade astrocytoma; Grade III—anaplastic astrocytoma, Grade IV—glioblastoma), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumor, primitive neuroectodermal (PNET), juvenile pilocytic astrocytoma (JPA), pineal tumor, and rhabdoid tumor.

In some embodiments, the cancer is a glioma. Gliomas may be further categorized based on the type of cells affected. In some embodiments, the cancer is a glioma. In some embodiments, the glioma is selected from the group consisting of: high grade glioma, diffuse astrocytoma, oligodendroglioma, oligoastrocytoma, secondary glioblastoma, and primary glioblastoma. In some embodiments, the cancer is a glioma. In some embodiments, the glioma is a high grade glioma, diffuse astrocytoma, oligodendroglioma, oligoastrocytoma, secondary glioblastoma, primary glioblastoma, or a diffuse intrinsic pontine glioma.

As will be evident to one or ordinary skill in the art, the amino acids within a protein may be referred to using the amino acid position number within the protein. In general, the amino acid position number can be identified by comparing the amino acid sequence of the protein, such as a variant protein, to the amino acid sequence of a reference protein (e.g., a wildtype protein). As used herein, the term “wildtype” may be used to refer to the nucleotide sequence or amino acid sequence of a protein found in the general population of subjects or with the highest frequency in a population. The term “wildtype allele” refers to an allele of a gene that is not associated a disease, such as a cancer, relative to a mutant allele, which may be more highly associated with the disease.

As also used herein, the term “mutant allele” refers to an allele of a gene that contains one or more mutations (e.g., substitutions, insertions, deletions) relative to a wildtype allele. In some embodiments, the mutant allele is associated with a disease, such as a cancer. As will be evident to one of skill in the art, a mutation to a nucleotide sequence of a gene may result in a mutation to the amino acid sequence of a protein encoded by the gene. Such mutated proteins may be referred to as variants. Generally, a protein variant may contain one or more (e.g., 1, 2, 3, 4, 5, or more) amino acid differences (mutations) relative to a wildtype protein. In some embodiments, the one or more amino acid mutations of the amino acid sequence of a protein may result in a change in the function of the protein. In some embodiments, one or more amino acid mutations to the amino acid sequence of a protein increases a function of the protein. In some embodiments, one or more amino acid mutations to the amino acid sequence of a protein reduces or eliminates a function of the protein. In some embodiments, the presence of a mutant allele may cause (directly or indirectly) a disease and therefore may be referred to a “causative mutation” or a “driving mutation.”

Alternatively or in addition, mutations may be present in the non-protein coding region of a nucleic acid, for example in a regulatory region, such as a promoter. In such cases, the mutated region may also be referred to as a variant, such as a promoter variant. Regulatory region variants, such as promoter variants, may affect regulation of the coding sequence of the nucleic acid. For example, mutation of one or more (e.g., 1, 2, 3, 4, 5, or more) nucleotides in a promoter region may affect expression of a coding sequence that is operably linked to the promoter region. In some embodiments, mutation of one or more nucleotides in a promoter region may affect binding of a transcription factor and therefore affect expression of the coding sequence. In some embodiments, one or more nucleotide mutations in a promoter region may increase transcription factor binding and increase expression of a coding sequence. In some embodiments, one or more nucleotide mutations in a promoter region may reduce or prevent transcription factor binding and reduce or prevent expression of the coding sequence.

In some embodiments, the one or more mutation is a single nucleotide polymorphism. As used herein, a “single nucleotide polymorphism” or “SNP” refers to a single substitution of a nucleotide for another nucleotide at a particular site that occurs in more than 1% of the general population. In some embodiments, the one or more mutation is a somatic mutation. As also used herein, the term “somatic mutation” refers to a mutation that occurs in any cell of the body other than germ cells, and is neither inherited from a parent nor passed to offspring.

In some embodiments, the cancer is characterized by a mutation in a gene encoding isocitrate dehydrogenase 1 (IDH1) or isocitrate dehydrogenase 2 (IDH2). In some embodiments, the cancer is characterized by the presence of an IDH1 protein variant containing a mutation at amino acid position R132, such as IDH1 variant R132H, IDH1 variant R132C, IDH1 variant R132G, IDH1 variant R132S, or IDH1 variant R132L.

In some embodiments, the cancer is characterized by a mutation in a regulatory sequence associated with the gene encoding telomerase reverse transcriptase (TERT), such as the TERT promoter. In some embodiments, the cancer is characterized by the presence of a TERT promoter variant containing a mutation on chromosome 5 at position 1,295,118 and 1,295,250, referred to as TERT promoter variant C228T and C250T, respectively.

In some embodiments, the cancer is characterized by a mutation in a gene encoding histone H3.3 (H3F3A). In some embodiments, the cancer is characterized by the presence of a histone H3.3 variant containing a mutation at amino acid position K27, such as K27M (referred to as H3F3A protein variant K27M).

In some embodiments, the cancer is characterized by a mutation in a gene encoding the serine threonine-protein kinase B-Raf (BRAF). In some embodiments, the cancer is characterized by the presence of B-Raf variant containing a mutation at amino acid position V600, such as V600E (referred to as BRAF protein variant V600E).

### Diagnostic Methods

Aspects of the present disclosure provide diagnostic methods (assays) for the identifying the genotype of cells. Such genotype identification allows for selection of an appropriate agent for therapeutic administration to the subject, for example during the course of a surgical procedure. The diagnostic methods described herein provide genotype information during an intraoperative timeframe, thereby allowing selection and administration of an appropriate therapeutic regimen. Accordingly, the diagnostic methods described herein may be characterized as “rapid,” referring to the ability to determine a the genotype of cells during the course of a surgical procedure.

Aspects of the present disclosure provide diagnostic methods (assays) that allow for the identification of the genotype of cells within a sample during an intraoperative timeframe (e.g, during a surgical procedure). The rapid genotype identification, such as the presence of one or more mutations in a target gene), allows for the selection of a therapeutic agent that may be effective in inhibiting growth and/or replication of cells having the genotype, specifically killing cells having the genotype, reducing metastasis of cells having the genotype, and/or reducing recurrence of the disease (cancer).

In some embodiments, the genotype of cells within the sample is determined during performance of a surgical procedure, such that a therapeutic agent may be selected and also administered during the surgical procedure. In some embodiments, the intraoperative timeframe is less than 8 hours. In some embodiments, the intraoperative timeframe is less than about 7, 6, 5, 4, 3, 2 hours, 1 hour, 55 minutes, 50 minutes, 45 minutes, 40 minutes, 35 minutes, or 30 minutes or less.

The rapid diagnostic methods to determine the genotype of cells in the sample obtained from the subject may be performed within the intraoperative timeframe. Diagnostics methods for genotype identification are known in the art, see, e.g., PCT Publication WO 2016/106391 and Shankar et al. JAMA Oncol. (2015) 1(5): 662-667, which are incorporated herein by reference in their entireties. However, the rapid diagnostic assays described herein allow for a more rapid and sensitive determination. In some embodiments, the rapid diagnostic assays allow for the determination of the genotype of cells in the sample within less than 1 hour. In some embodiments, the rapid assays described herein allow for the determination of the genotype of cells in the sample within less than 30 minutes. In some embodiments, the rapid assays allow for the determination of the genotype of cells in the sample in about 60, 59, 58, 57, 56, 55, 54, 53, 52, 51, 50, 50, 49, 48, 47, 46, 45, 44, 43, 42, 41, 40, 39, 38, 37, 36, 35, 34, 33, 32, 31, 30, 29, 28, 27, 26, 25 minutes or less.

The diagnostic methods described herein generally involve obtaining a sample from the subject and detecting one or more mutations in a target nucleic acid (e.g., a gene). In some embodiments, the sample is obtained from the subject during performance of a surgical procedure, such as a surgical procedure to remove cancer cells (e.g., a surgical resectioning procedure). In some embodiments, the sample is a tissue, blood, plasma, serum, tissue, or cerebrospinal fluid sample obtained from the subject. In some embodiments, the sample is a portion of a tumor or cancer cells removed from a subject, such as during a surgical resectioning procedure.

The rapid diagnostic methods described herein involve detecting one or more mutations in a target nucleic acid in a sample obtained from the subject or in a nucleic acid isolated from the sample. As used herein, the target nucleic acid is a region of a nucleic acid in which a mutation of interest may be located, if present. For example, in methods in which detection of a IDH1 R132 mutation is desired, the target nucleic acid may be a region of a chromosome containing the gene encoding IDH1, the gene encoding IDH1, or a portion of the gene encoding IDH1. In some embodiments, the nucleotide sequence of the target nucleic acid encodes a wildtype allele. In some embodiments, the nucleotide sequence of the target nucleic acid encodes a mutant allele, such as any of the mutations described herein. In some embodiments, a sample obtained from subject or nucleic acid isolated from the sample may contain both a wildtype allele and a mutant allele. In such instances, it is desired that the mutant allele is selectively amplified for detection and identification using the methods described herein, which may involve blocking or preventing amplification of the wildtype allele.

As described herein, the rapid diagnostic methods may be useful in detecting mutations associated with a disease (e.g., cancer), for example mutations in IDH1/2, TERT promoter, H3F3A, and/or BRAF. In some embodiments, the rapid diagnostic methods detect a mutant allele of IDH/21, TERT promoter, H3F3A, and/or BRAF, which may encode a variant of any of the aforementioned genes or proteins. In general, detection of a genotype (e.g., a mutant allele) in a sample from a subject, wherein the genotype is associated with a disease (e.g., cancer), may be indicative that the subject from which the sample was obtained has cancer or a particular type of cancer. In some embodiments, the identification of the genotype may allow a surgeon or other practitioner to select a treatment or suitable therapeutic agent. For example, detection of a mutant allele encoding a IDH1 protein variant (e.g., R132H, R132C, R132G, R132S, or R132L) may indicate that the subject has a cancer characterized by a IDH1 protein variant, and therefore, a therapeutic agent that is effective towards IDH1 protein variant cancers is selected for local administration, as described herein. Accordingly, such genotype identification during an intraoperative timeframe allows a surgeon (or other practitioner) to select an appropriate therapeutic agent and locally administer the therapeutic agent during the course of the procedure. In some embodiments, the presence of a mutant allele allows for selection of an appropriate therapeutic agent.

In some embodiments, the amount of the mutant allele in a sample is determined. In some embodiments, the amount of the mutant allele in a sample is compared to the amount of the wildtype allele in the sample. In some embodiments, an amount of the mutant allele in the sample that is higher than the amount of the wildtype allele in the sample indicates that the subject has cancer or has a particular type of cancer. In some embodiments, if the amount of the mutant allele in the sample is higher than the amount of the wildtype allele in the sample, an appropriate therapeutic agent is selected and locally administered to the subject.

In some embodiments, the amount of the mutant allele in a sample is compared to the amount of the mutant allele in a reference (e.g., control) sample. In some embodiments, an amount of the mutant allele in the sample that is higher than the amount of the mutant allele in a reference sample indicates that the subject has cancer or has a particular type of cancer. In some embodiments, if the amount of the mutant allele in the sample is higher than the amount of the mutant allele in a reference sample, an appropriate therapeutic agent is selected and locally administered to the subject.

Detecting one or more mutations or mutant genotypes in a target nucleic acid generally involves isolating nucleic acid from a sample obtained from a subject and analyzing the nucleic acid for the presence of one or more allele-specific mutations, such as those described herein. Methods of isolating nucleic acid from a sample, such as a tissue, blood, plasma, serum, tissue, or cerebrospinal fluid sample, obtained from the subject are known in the art and may be suitable for use in the rapid diagnostic methods described herein. In some embodiments, isolating nucleic acid from a sample involves extraction of nucleic acid from cells and/or other components of the sample. In some embodiments, the method further involves purifying the nucleic acid from cells and/or other components of the sample. In some embodiments, nucleic acid is isolated from the sample using a rapid nucleic acid isolation or extraction method. The nucleic acid isolated from the sample may be DNA or RNA.

In some embodiments, nucleic acid isolated from a sample is analyzed using a polymerase chain reaction. As will be evident to one of ordinary skill in the art, polymerase chain reactions (PCR) generally involve a cyclic program of two or more steps performed at distinct temperatures.

In some embodiments, the PCR reaction is a quantitative PCR (qPCR), which couples the PCR reaction with quantification of the amplified product. Quantitative PCR can be used to determine whether a particular sequence (e.g., a mutant allele) is present in a sample, and to determine the number of its copies in the sample. Quantitative PCR often methods use fluorescent dyes, such as Sybr Green, EvaGreen or fluorophore-containing DNA probes, such as TaqMan, to measure the amount of amplified product throughout the course of the reaction.

As described for example, in PCT Publication WO 2016/106391, PCR reactions, including qPCR reactions, generally include an initial step, a denaturation step, an annealing step, and an extension/elongation step (also referred to as an amplification step), although it may not be necessary to include all of the aforementioned steps. An initial step may involve heating the reaction. The denaturing step involves heating the reaction in order to denature (melt) the nucleic acid causing separation of the nucleic acid strands. The annealing step allows for the forward and reverse primers to anneal to complementary target nucleic acid. This interaction is facilitated by hydrogen bonds between the nucleotides of each of the forward primer and reverse primer and their corresponding complementary nucleotide sequence in the denatured nucleic acid, if present. The polymerase then binds to the hybridized primer and template and begins DNA polymerization. If the corresponding complementary nucleotide sequence(s) are not present in denatured nucleic acid, annealing and polymerization will not occur or will occur at a slower rate. The extension/elongation step involves DNA polymerase synthesis of a new DNA strand complementary to the strand to which the primer annealed (template strand), by adding dNTPs that are complementary to the template strand in 5′ to 3′ direction, condensing the 5′-phosphate group of the dNTPs with the 3′-hydroxyl group at the end of the nascent (extending) DNA strand.

In some embodiments, the denaturing step involves incubating the reaction at 94-98° C. for 1-12 seconds. In some embodiments, the denaturing step involves incubating the reaction at 94-98° C. for 1, 2, 3, 4, 5, 6, 7, 8, 9, 10, 11, or 12 seconds. In some embodiments, the denaturing step involves incubating the reaction at 94-98° C. for 5 seconds.

In some embodiments, the annealing step involves incubating the reaction at 50-65° C. for 5-25 seconds. In some embodiments, the annealing step involves incubating the reaction at 50-65° C. for 5, 6, 7, 8, 9, 10, 11, 12, 13, 14, 15, 16, 17, 18, 19, 20, 21, 21, 22, 23, 24, or 25 seconds. In some embodiments, the denaturing step involves incubating the reaction at 50-65° C. for 10 seconds. Selection for the temperature for the annealing step will depend on factors such as the melting temperature of the primers, probes, and PNA blockers. As will be evident to one of ordinary skill in the art, the melting temperature (Tm) will vary based on the length of the nucleotide sequence (of the primer, probes, and PNA blocker) and the nucleotide composition (e.g., the percentage of the nucleotides that are guanines or cytosines (G-C content)). In some embodiments, the melting temperature of the primers, probes, and PNA blockers is within a range of 50° C. to 65° C., 62.5° C. to 64.5° C., or 63.0° C. to 64.0° C. In some embodiments, the melting temperature of the forward primer is 63.5° C. In some embodiments, the annealing temperature is 63.5° C.

In some embodiments, the elongation/extension step involves incubating the reaction at 62.5-64.5° C. for 5-25 seconds. In some embodiments, the elongation/extension step involves incubating the reaction at 62.5-64.5° C. for 5, 6, 7, 8, 9, 10, 11, 12, 13, 14, 15, 16, 17, 18, 19, 20, 21, 21, 22, 23, 24, or 25 seconds. In some embodiments, the denaturing step involves incubating the reaction at 50-65° C. for 10 seconds. The length of the extension/elongation step depends both on the DNA polymerase used and on the length of the DNA fragment to be amplified.

In some embodiments, the annealing and elongation/extension steps are performed together. In some embodiments, the annealing and elongation/extension steps are performed at the same temperature and accordingly, may occur concurrently in the reaction.

The PCR reaction proceeds through multiple cycles of the denaturation, annealing, and extension/elongation steps. In some embodiments, the annealing and elongation/extension steps are performed together and the PCR reaction proceeds through multiple cycles of the denaturation step and concurrent annealing and extension/elongation steps. In some embodiments, the PCR reaction proceeds through a series of 20-50 cycles. In some embodiments, the PCR reaction proceeds through a series of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 cycles. In some the PCR reaction proceeds through a series of 40 cycles.

The PCR reactions described herein involve annealing a forward primer comprising a nucleotide sequence that hybridizes (is complementary to) a first region on the sense strand of the target nucleic acid and a reverse primer comprising a nucleotide sequence that hybridizes (is complementary to) a second region on the antisense strand of the target nucleic acid. As used herein, a “forward primer” refers to a short, single-stranded nucleotide sequence that hybridizes to a target nucleic acid and enables addition of new deoxyribonucleotides by DNA polymerase at the 3′ end, wherein the primer binds upstream of a target site and to the sense strand of the target nucleic acid. In some embodiments, the forward primer is 18-35, 19-32, or 21-31 nucleotides (inclusive) in length. In some embodiments, the melting temperature of the forward primer is within a range of 50° C. to 65° C., 62.5° C. to 64.5° C., or 63.0° C. to 64.0° C. In some embodiments, the melting temperature of the forward primer is 63.5° C.

As used herein, a “reverse primer” refers to a short, single-stranded nucleotide sequence that hybridizes to a target nucleic acid and enables addition of new deoxyribonucleotides by DNA polymerase at the 3′ end, wherein the primer binds downstream of a target site and to the antisense strand of the target nucleic acid. In some embodiments, the reverse primer is 18-35, 19-32 or 21-31 nucleotides (inclusive) in length. In some embodiments, the melting temperature of the reverse primer is within a range of 50° C. to 65° C., 62.5° C. to 64.5° C., or 63.0° C. to 64.0° C. In some embodiments, the melting temperature of the reverse primer is 63.5° C.

In general, the forward primer and reverse primer will anneal to regions within the target nucleic acid and amplify a DNA amplicon containing the mutant allele, if present. A DNA amplicon containing a mutant allele may be detected within the PCR reaction, for example by binding with a probe.

Examples of forward primers and reverse primers for use in amplifying and detecting mutant alleles in IDH1, TERT promoter, H3F3A, and BRAF, as described herein, are provided in Table 2.

The rapid diagnostic methods described herein also involve annealing a PNA blocker that hybridizes to a wildtype allele of the target nucleic acid, if present. As used herein, the terms “PNA blocker” and “peptide nucleic acid blocker” are used to refer to artificial, chemically synthesized nucleic acid analogues which have a structure in which a basic backbone of a nucleic acid constituted by pentose and phosphoric acid is replaced with a polyamide backbone having no charge including glycine as units. Unlike primers, PNA blockers do not prime or imitate DNA polymerization. The PNA blockers described herein hybridize to the wildtype allele of a target nucleic acid, if present, and therefore, block (clamp) amplification of the wildtype allele. The presence of the PNA blockers allow for the selective amplification of mutant alleles from a population of target nucleic acids that may contain both mutant alleles and wildtype alleles. In some embodiments, the PNA blocker hybridizes to a nucleotide sequence located between the region amplified by the forward primer and reverse primer.

In some embodiments, the PNA blocker is 10-25, 12-22, or 12-19 nucleotides (inclusive) in length. In some embodiments, the melting temperature of the PNA blocker is within a range of 50° C. to 65° C., 62.5° C. to 64.5° C., or 63.0° C. to 64.0° C. In some embodiments, the melting temperature of the PNA blocker primer is 63.5° C.

Examples of PNA blockers that bind and block (clamp) wildtype alleles of IDH1, TERT promoter, H3F3A, and BRAF, as described herein, are provided in Table 2.

The rapid diagnostic methods described herein also involve annealing a probe that is complementary to a mutant allele sequence in the target nucleic acid, if present. As used herein, the term “probe” is used to refer to a nucleic acid containing a sequence that is capable of hybridizing to a mutant allele. In some embodiments, the probe further comprises a cleavable fluorescent moiety that, upon cleavage, emits a fluorescent signal that serves as an indicator for the presence of the specific mutant allele. In some embodiments, the fluorescent moiety is cleaved by the polymerase in the PCR reaction, thereby releasing the fluorescent signal which can then be detected. An example of probes for use in the methods described herein are locked nucleic acid (LNA) probes. LNA probes are artificial, chemically synthesized nucleic acid analogues comprising an RNA nucleoside in which the 2′ oxygen and 4′ carbon of the ribose moiety are chemically linked (e.g., by a methylene bond). Like primers, LNA probes may act as a primer for nucleic acid polymerases and therefore become incorporated into a nucleic acid.

In some embodiments, the probe (e.g., LNA probe) is 10-50, 15-45, 10-25, or 20-40 nucleotides (inclusive) in length. In some embodiments, the melting temperature of the probe (e.g., LNA probe) is within a range of 50° C. to 65° C., 62.5° C. to 64.5° C., or 63.0° C. to 64.0° C. In some embodiments, the melting temperature of the reverse primer is 63.5° C.

Examples of probes (e.g., LNA probes) that bind mutant alleles of IDH1, TERT promoter, H3F3A, and BRAF, as described herein, are provided in Table 2.

The PCR reactions described herein may also comprise one or more buffers as described herein but not limited to a PCR buffer. In general, PCR buffers may include Tris-HCl, KCl, and MgCl₂. In some embodiments, the PCR reaction may also comprise one or more additional components for performing the methods. In some embodiments, the PCR reaction may also comprise a deoxynucleotide triphosphate solution mix (dNTPs). In some embodiments, the PCR reaction may also comprise polymerase, such as a DNA polymerase. In general, DNA polymerases suitable for use in the assay methods described herein are known in the art and may be commercially available. Selection of such components (e.g., PCR buffers, dNTPs, DNA polymerases) will be evident to one or ordinary skill in the art.

In some embodiments, the rapid diagnostic method is multiplexed, meaning the method may involve detection of more than one mutation in a single reaction. In some embodiments, the polymerase chain reaction may contain primers, probes, and/or PNA blockers specific for more than one mutation, thereby allowing detection of more than one mutation in a single reaction. In some embodiments, the polymerase chain reaction may contain primers and probes for detecting a control gene in addition to primers and probes for detecting one or more mutation, thereby allowing detection of a control gene and one or more mutations in a single reaction.

Inclusion of a primers and probes for detecting a control gene provides a means by which to assess the integrity of the assay method (an internal control). For example, amplification and detection of the control gene in the absence of amplification and detection of a mutant allele ensures the method functioned properly and the mutant allele was not amplified and detected due to absence of the mutant allele or presence at an allelic fraction below the limit of detection. In some embodiments, the control gene is a housekeeping gene, such as a gene that is considered to be generally required or present or the maintenance of basic cellular functions. In some embodiments, the control gene is GADPH (glyceraldehyde 3-phosphate dehydrogenase).

The rapid diagnostic methods described herein provide highly sensitive methods for detecting of mutant alleles. In some embodiments, the methods allows for detection of a mutant allele that is present at less than 10% of the allelic fraction (10% of the mutant allele and 90% of the wildtype allele). In some embodiments, the methods allows for detection of a mutant allele that is present at less than 10%, 9%, 8%, 7%, 6%, 5%, 4%, 3%, 2%, 1%, 0.9%, 0.8%, 0.7%, 0.6%. 0.5%, or less of the allelic fraction. In some embodiments, the methods allows for detection of a mutant allele that is present about 1% of the allelic fraction.

In some embodiments, the diagnostic methods are for detecting a mutation in IDH1 or for detecting mutations which may include a mutation in IDH1. In some embodiments, the method includes use of a set of primers for amplification of a mutant allele of IDH1 (a IDH1 variant), if present in the sample. In some embodiments, the method includes use of more than one sets of primers for amplification of more than one mutant allele (e.g., R132H, R132C, R132G, R132S, R132L) of IDH1 (a IDH1 variant), if present in the sample. In some embodiments, method includes use of a probe for detection of a mutant allele of IDH1 (a IDH1 variant). In some embodiments, the method includes use of a PNA blocker that clamps (blocks) the wildtype nucleotide sequence and prevents amplification of a wildtype allele.

In some embodiments, the diagnostic methods are for detecting a IDH1 R132H variant or for detecting mutations which may include a IDH1 R132H variant. In some embodiments, the method includes use of a forward primer having a nucleotide sequence set forth by SEQ ID NO: 2 and a reverse primer having a nucleotide sequence set forth by SEQ ID NO: 18. In some embodiments, the method includes use of a probe having a nucleotide sequence set forth by SEQ ID NO: 9. In some embodiments, the method includes use of a PNA blocker having a nucleotide sequence set forth by SEQ ID NO: 24.

In some embodiments, the diagnostic methods are for detecting a IDH1 R132C variant or for detecting mutations which may include a IDH1 R132C variant. In some embodiments, the method includes use of a forward primer having a nucleotide sequence set forth by SEQ ID NO: 3 and a reverse primer having a nucleotide sequence set forth by SEQ ID NO: 18. In some embodiments, the method includes use of a probe having a nucleotide sequence set forth by SEQ ID NO: 10. In some embodiments, the method includes use of a PNA blocker having a nucleotide sequence set forth by SEQ ID NO: 24.

In some embodiments, the diagnostic methods are for detecting a IDH1 R132G variant or for detecting mutations which may include a IDH1 R132G variant. In some embodiments, the method includes use a forward primer having a nucleotide sequence set forth by SEQ ID NO: 3 and a reverse primer having a nucleotide sequence set forth by SEQ ID NO: 18. In some embodiments, the method includes use of a probe having a nucleotide sequence set forth by SEQ ID NO: 11. In some embodiments, the method includes use of a PNA blocker having a nucleotide sequence set forth by SEQ ID NO: 24.

In some embodiments, the diagnostic methods are for detecting a IDH1 R132S variant or for detecting mutations which may include a IDH1 R132S variant. In some embodiments, the method includes use of a forward primer having a nucleotide sequence set forth by SEQ ID NO: 3 and a reverse primer having a nucleotide sequence set forth by SEQ ID NO: 18. In some embodiments, the method includes use of a probe having a nucleotide sequence set forth by SEQ ID NO: 12. In some embodiments, the method includes use of a PNA blocker having a nucleotide sequence set forth by SEQ ID NO: 24.

In some embodiments, the diagnostic methods are for detecting a IDH1 R132L variant or for detecting mutations which may include a IDH1 R132L variant. In some embodiments, the method includes use of a forward primer having a nucleotide sequence set forth by SEQ ID NO: 3 and a reverse primer having a nucleotide sequence set forth by SEQ ID NO: 18. In some embodiments, the method includes use of a probe having a nucleotide sequence set forth by SEQ ID NO: 13. In some embodiments, the method includes use of a PNA blocker having a nucleotide sequence set forth by SEQ ID NO: 24.

In some embodiments, the diagnostic methods are for detecting a mutation in the TERT promoter or for detecting mutations which may include a mutation in the TERT promoter. In some embodiments, the method includes use of a set of primers for amplification of a mutant allele of the TERT promoter (a TERT variant), if present in the sample. In some embodiments, the method includes use of more than one sets of primers for amplification of more than one mutant allele (e.g., C228T, C250T) of the TERT promoter (a TERT variant), if present in the sample. In some embodiments, the method includes use of a probe for detection of a mutant allele of the TERT promoter (a TERT variant). In some embodiments, the method includes use of a PNA blocker that clamps (blocks) the wildtype nucleotide sequence and prevents amplification of a wildtype allele.

In some embodiments, the diagnostic methods are for detecting a TERT promoter variant TERT C228T or for detecting mutations which may include a detecting a TERT C228T variant. In some embodiments, the method includes use of a forward primer having a nucleotide sequence set forth by SEQ ID NO: 1 and a reverse primer having a nucleotide sequence set forth by SEQ ID NO: 17. In some embodiments, the method includes use of a probe having a nucleotide sequence set forth by SEQ ID NO: 7. In some embodiments, the method includes use of a PNA blocker having a nucleotide sequence set forth by SEQ ID NO: 22.

In some embodiments, the diagnostic methods are for detecting a TERT promoter variant TERT C250T or for detecting mutations which may include a TERT C250T variant. In some embodiments, the method includes use of a forward primer having a nucleotide sequence set forth by SEQ ID NO: 1 and a reverse primer having a nucleotide sequence set forth by SEQ ID NO: 17. In some embodiments, the method includes use of a probe having a nucleotide sequence set forth by SEQ ID NO: 8. In some embodiments, the method includes use of a PNA blocker having a nucleotide sequence set forth by SEQ ID NO: 23.

In some embodiments, the diagnostic methods are for detecting a mutation in H3F3A for detecting mutations which may include a mutation in H3F3A. In some embodiments, the method includes use of a set of primers for amplification of a mutant allele of H3F3A (a H3F3A variant), if present in the sample. In some embodiments, the method includes use of more than one sets of primers for amplification of more than one mutant allele of the H3F3A promoter (a H3F3A variant), if present in the sample. In some embodiments, the method includes use a probe for detection of a mutant allele of H3F3A (a H3F3A variant). In some embodiments, the method includes use of a PNA blocker that clamps (blocks) the wildtype nucleotide sequence and prevents amplification of a wildtype allele.

### Formulations for Local Administration

In some embodiments, the composition may comprise a carrier. The carrier may comprise e.g., a hydrogel, a particle, a wafer, and/or a suitable implant. The carrier may be a pharmaceutically-acceptable carrier. The carrier may comprise as non-limiting examples a biocompatible polymer, a biodegradable polymer, and inorganic material, an organic material, a synthetic material, and naturally-derived material, a metal (e.g., gold), a nonmetal, a transition metal (e.g., iron), a ceramic, silica, and/or a carbon allotrope (e.g., graphene, diamond, carbon nanotube), or a combination thereof. The biocompatible polymer and/or biodegradable polymer may comprise for example poly(lactic-co-glycolic acid) copolymers (PLGA), chitosan, poly(L-lactic acid), a poly(glycolic acid), poly(ethylene glycol), poly(L-glutamic acid), poly(L-lysine), poly(L-arginine), poly(L-histidine), poly acrylic acid, polyethylenimine, alginate, polyvinyl chloride, polytetrafluoroethylene, polyethersulfone, polyethylene, polysulfone, polyether ether ketone, polypropylene, a phosphorus-containing polymer (e.g., a polyphosphazene, a polyphosphoester), a polyanhydride (e.g., polyanhydride-imide, polyanhydride-ester), a polyacetal, a poly(ortho ester), a polyurethane, a polylactide, a polycarbonate, a polyamide (e.g., polyiminocarbonate), a polyacrylamide (e.g., poly N-isopropylacrylamide), a poly(beta amino ester), a poly(alpha ester), a polyester (e.g., polycaprolactone), and/or poly(propylene fumarate), or a combination thereof. See, e.g., Langer, R. S. et al., “Polymeric Systems for Controlled Drug Release,” Chem. Rev. 1999, 99, 3181-3198. In some embodiments, the carrier (e.g., particles, e.g., polymeric microparticles) comprises PLGA.

According to some embodiments, the carrier (e.g., particles) may comprise a polymer. Generally, polymers are materials comprising three or more repeating units in their chemical structure. Polymers may comprise additional repeating units and may have any molecular weight. In some embodiments, the carrier (e.g., particle) may comprise polymers that are in the form of fibers, or may comprise polymeric fibers.

In some embodiments, the polymer of the carrier (e.g., particle) has a number average molecular weight of greater than or equal to 1,000 Da, greater than or equal to 5,000 Da, greater than or equal to 10,000 Da, greater than or equal to 25,000 Da, greater than or equal to 50,000 Da, greater than or equal to 100,000 Da, or greater than or equal to 500,000 Da. The polymer in the carrier (e.g., particle) may have a number average molecular weight less than or equal to 1,000,000 Da, less than or equal to 500,000 Da, less than or equal to 100,000 Da, less than or equal to 50,000 Da, less than or equal to 25,000 Da, less than or equal to 10,000 Da, or less than or equal to 5,000 Da. Combinations of the above-referenced ranges are also possible (e.g., greater than or equal to 10,000 Da and less than or equal to 50,000 Da). Other ranges are also possible.

In certain embodiments, the polymer of the carrier (e.g., particle) has a weight average molecular weight of greater than or equal to 1,000 Da, greater than or equal to 5,000 Da, greater than or equal to 10,000 Da, greater than or equal to 25,000 Da, greater than or equal to 50,000 Da, greater than or equal to 100,000 Da, or greater than or equal to 500,000 Da. The polymer in the carrier (e.g., particle) may have a weight average molecular weight less than or equal to 1,000,000 Da, less than or equal to 500,000 Da, less than or equal to 100,000 Da, less than or equal to 50,000 Da, less than or equal to 25,000 Da, less than or equal to 10,000 Da, or less than or equal to 5,000 Da. Combinations of the above-referenced ranges are also possible (e.g., greater than or equal to 10,000 Da and less than or equal to 50,000 Da). Other ranges are also possible.

The polymer in the particle may have any chain structure in accordance with certain embodiments. In some embodiments, polymers may be linear, branched, and/or crosslinked molecules. Or they may be in the form of a crosslinked network. Polymers may have branches or crosslinks of any molecular weight, functionality, and/or spacing. In accordance with some embodiments, the polymers may be highly monodisperse. In accordance with other embodiments, the polymers may be polydisperse. One of ordinary skill in the art would be aware of methods for dispersity. For example, dispersity can be assessed by size-exclusion chromatography and/or light scattering.

Polymeric carriers (e.g., particles, microparticles) may have any desired mechanical property. In certain embodiments, polymeric carriers (e.g., particles, microparticles) are rubbery, glassy, and/or semicrystalline.

In some embodiments, the polymers can be homopolymers, blends of polymers, and/or copolymers. Copolymers may be random copolymers, tapered copolymers, and block copolymers. In certain embodiments, block copolymers with more than three blocks may comprise two or more blocks formed from the same monomer. Blends of polymers can be phase separated or single phase, according to some embodiments.

In some embodiments, polymers may be organic polymers, inorganic polymers, or organometallic polymers. It may be advantageous, according to certain but not necessarily all embodiments, for the carrier (e.g., particle) to comprise an organic polymer material. In such embodiments, at least 50%, at least 75%, at least 90%, at least 95%, or at least 990%, or 100% of the carrier (e.g., polymeric particle) is made up of organic polymer material. In certain embodiments, polymers are of synthetic origin. Polymers of synthetic origin may be formed by either step growth or chain growth processes, and may be further functionalized after polymerization. Non-limiting examples of suitable polymers include polystyrene, polyethylene, polypropylene, poly(methyl methacrylate), polyacrylonitrile, polybutadiene, polyisoprene, poly(dimethyl siloxane), poly(vinyl chloride), poly(tetrafluoroethylene), polychloroprene, poly(vinyl alcohol), poly(ethylene oxide), polycarbonate, polyester, polyamide, polyimide, polyurethane, poly(ethylene terephthalate), polymerized phenol-formaldehyde resin, polymerized epoxy resin, para-amid fibers, silk, collagen, keratin, and gelatin. Additional examples of suitable polymers that can be used include, but are not limited to, relatively high temperature fluoropolymers (e.g., Teflon®), polyetherether ketone (PEEK), and polyether ketone (PEK). In some embodiments, the polymer is not a polyelectrolyte.

In some embodiments, the carrier (e.g., polymeric particles) may further comprise additives. The carrier (e.g., polymeric particles) may be in the form of a gel and comprise solvent, according to certain embodiments. In some embodiments, the carrier (e.g., polymeric particles) may comprise one or more of fillers, additives, plasticizers, small molecules, and particles comprising ceramic and/or metal. In certain embodiments, greater than or equal to 50%, greater than or equal to 80%, greater than or equal to 90%, greater than or equal to 95%, or greater than or equal to 99% of the mass of the carrier (e.g., polymeric particle) may comprise polymers. Other ranges are also possible.

In some embodiments, the composition comprises an implant. The implant may have a maximum dimension (e.g., length or diameter) from 0.1 microns to 20 mm. For example, the implant may have a maximum length greater than about 0.1 microns and less than about 15 mm.

The produced implants can be in the form of rods, cylinders, rings, discs, ellipses, spheres, random particle shapes, cubes, and the like. For some examples of suitable implants, see U.S. Publication No.: US 2014/0336164 A1, incorporated herein by reference.

In some embodiments, the composition comprises a population of particles, e.g. microparticles. Generally, the term “microparticle” is used to refer to any particle having a maximum cross-sectional dimension of greater than or equal to 0.1 microns and less than or equal to 100 microns (see, e.g., IUPAC definition). In some embodiments, the microparticle has a maximum cross-sectional dimension of less than or equal to 100 microns, less than or equal to 50 microns, less than or equal to 10 microns, less than or equal to 9 microns, less than or equal to 8 microns, less than or equal to 7 microns, less than or equal to 6 microns, less than or equal to 5 microns, or less than or equal to 4 microns. In some embodiments, the microparticle has a maximum cross-sectional dimension of at least 0.2 microns, at least 0.5 microns, at least 0.8 microns, at least 1 microns, at least 2 microns, at least 3 microns. In some embodiments, the microparticle has a maximum cross-sectional dimension of between 0.1 microns and 100 microns, between 1 microns and 10 microns, between 2 microns and 5 microns, or between 3 microns and 4 microns. Other combinations of the above-referenced ranges are also possible. Other values are also possible.

In some embodiments, the mean diameter of the population of microparticles is greater than or equal to 0.1 microns and less than or equal to 100 microns. In some embodiments, the mean diameter of the population of microparticles is less than or equal to 100 microns, less than or equal to 50 microns, less than or equal to 10 microns, less than or equal to 9 microns, less than or equal to 8 microns, less than or equal to 7 microns, less than or equal to 6 microns, less than or equal to 5 microns, or less than or equal to 4 microns. In some embodiments, the mean diameter of the population of microparticles is at least 0.2 microns, at least 0.5 microns, at least 0.8 microns, at least 1 microns, at least 2 microns, at least 3 microns. In some embodiments, the mean diameter of the population of microparticles is between 0.1 microns and 100 microns, between 1 microns and 10 microns, between 2 microns and 5 microns, or between 3 microns and 4 microns. Other combinations of the above-referenced ranges are also possible. Other values are also possible.

In some embodiments, the particle is a nanoparticle. Generally, the term “nanoparticle” is used to refer to any particle having a maximum cross-sectional dimension of less than 1 micron. In some embodiments, the microparticle has a maximum cross-sectional dimension of less than 500 nm, less than 250 nm, less than 100 nm, less than 10 nm, less than 5 nm, less than 3 nm, less than 2 nm, less than 1 nm, between 0.3 and 10 nm, between 10 nm and 100 nm, or between 100 nm and 1 micron.

In certain embodiments, a relatively large percentage of the particles have sizes falling within a certain range. For example, in some embodiments, at least 50%, at least 75%, at least 90%, at least 95%, at least 98%, or at least 99% of the volume occupied by the particles is made up of particles having maximum cross-sectional dimensions less than or equal to 100 microns, less than or equal to 50 microns, less than or equal to 10 microns, less than or equal to 9 microns, less than or equal to 8 microns, less than or equal to 7 microns, less than or equal to 6 microns, less than or equal to 5 microns, or less than or equal to 4 microns. In some embodiments, at least 50%, at least 75%, at least 90%, at least 95%, at least 98%, or at least 99% of the volume occupied by the particles is made up of particles having maximum cross-sectional dimensions of at least 0.2 microns, at least 0.5 microns, at least 0.8 microns, at least 1 microns, at least 2 microns, at least 3 microns. Combinations of these ranges are also possible. Other values are also possible.

As used herein, the “maximum cross-sectional dimension” of an object refers to the largest distance between two opposed boundaries of the object. The “average maximum cross-sectional dimension” of a plurality of objects refers to the number average. The average maximum cross-sectional dimension (D_{avg}) of n objects would be calculated as:

$${D}_{avg} = \frac{{\sum }_{i = 1}^{i = n} {D}_{i}}{n}$$

wherein Dᵢ is the maximum cross-sectional dimension of the i^{th} object.

According to certain embodiments, the particles (e.g., microparticles, polymeric microparticles, nanoparticles) may be substantially the same size (“monodisperse”). For example, the particles may have a distribution of dimensions such that the standard deviation of the maximum cross-sectional dimensions of the particles is no more than 50%, no more than 25%, no more than 10%, no more than 5%, no more than 2%, or no more than 1% of the average maximum cross-sectional dimension of the particles. Standard deviation (lower-case sigma) is given its normal meaning in the art, and may be calculated as:

$$σ = \sqrt{\frac{{\sum }_{i = 1}^{n} {( {D}_{i} - {D}_{avg} )}^{2}}{n - 1}}$$

wherein Dᵢ is the maximum cross-sectional dimension of particle i, D_{avg} is the average maximum cross-sectional dimension of the particles, and n is the number of particles. The percentage comparisons between the standard deviation and the average maximum cross-sectional dimensions of the particles outlined above can be obtained by dividing the standard deviation by the average and multiplying by 100%.

The particles may have a variety of suitable shapes. Non-limiting examples of suitable shapes include spheres, cylinders, cubes, and parallelepipeds. The particles may have any suitable cross-sectional shape including, but not limited to, circular, triangular, square, rectangular, pentagonal, hexagonal, heptagonal, octagonal, irregular, and the like.

In some embodiments, the composition may comprise a therapeutic agent. The composition may comprise one or more therapeutic agents, for example two or more agents, three or more agents, or for more agents. In some embodiments, the therapeutic agent comprises a metabolic inhibitor. In some embodiments, the therapeutic agent may comprise a nicotinamide adenine dinucleotide (NAD) biosynthesis inhibitor. In some embodiments, the NAD biosynthesis inhibitor is a nicotinamide phosphoribosyltransferase (NAMPT) inhibitor. In some cases, the NAMPT inhibitor is GMX-1778 or FK866.

The therapeutic agent (e.g., active substance) may be associated with the carrier (e.g., polymeric microparticle, polymer network, nanoparticle) and/or present in the composition in any suitable amount. The agent may be present in an amount from about 1% to 90% by weight of the composition. In some embodiments, the agent is present in an amount from about 20% to about 80% by weight of the composition. In some embodiments, the agent (e.g., active substance) is present in the composition in an amount ranging between about 0.01% weight/weight and about 50% weight/weight versus the total composition weight. In some embodiments, the agent (e.g., active substance) is present in the composition in an amount of at least about 0.01% weight/weight, at least about 0.05% weight/weight, at least about 0.1% weight/weight, at least about 0.5% weight/weight, at least about 1% weight/weight, at least about 2% weight/weight, at least about 3% weight/weight, at least about 5% weight/weight, at least about 10% weight/weight, at least about 15% weight/weight, at least about 20/o weight/weight, at least about 30% weight/weight, at least about 40% weight/weight of the total composition weight. In certain embodiments, the active substance is present in the composition in an amount of less than or equal to about 50% weight/weight, less than or equal to about 40% weight/weight, less than or equal to about 30% weight/weight, less than or equal to about 20% weight/weight, less than or equal to about 15% weight/weight, less than or equal to about 10% weight/weight, less than or equal to about 5% weight/weight, less than or equal to about 3% weight/weight, less than or equal to about 2% weight/weight, less than or equal to about 1% weight/weight, less than or equal to about 0.5% weight/weight, less than or equal to about 0.1% weight/weight, or less than or equal to about 0.05% weight/weight versus the total composition weight. Any and all closed ranges that have endpoints within any of the above-referenced ranges are also possible (e.g., between about 0.01% weight/weight and about 50% weight/weight, between about 0.01% weight/weight and about 15% weight/weight, between 1% weight/weight to 10% weight/weight). Other ranges are also possible.

In some embodiments, the load of the agent (e.g., the NAD biosynthesis inhibitor) is between 0.1% to 10% (weight/weight). In some embodiments, the load of the agent (e.g., the NAD biosynthesis inhibitor) is between 0.1% to 5%, 0.1% to 0.5%, 0.1% to 1%, 1% to 5%, 2% to 5%, 3% to 5%, 1% to 2%, 1% to 3%, 1% to 4%, 2% to 3%, 2% to 4%, 3% to 4%, 4% to 5%, 3% to 7%, 5% to 10%, 5% to 9%, 5% to 8%, 5% to 7%, 5% to 6%, 6% to 10%, 6% to 9%, 6% to 8%, 6% to 7%, 7% to 9%, 7% to 8%, or 7% to 10% (weight/weight). In some embodiments, the load of the agent (e.g., the NAD biosynthesis inhibitor) is between 4% to 5% (weight/weight). Advantageously, certain embodiments of the compositions described herein may permit higher concentrations (weight percent) of agents (e.g., active substances) such as therapeutic agents to be incorporated as compared to other carriers (e.g., polymers such as certain conventional hydrogel s).

According to some embodiments, the composition and methods described herein are compatible with one or more agents (e.g., therapeutic agents, diagnostic agents, and/or enhancement agents), such as drugs, nutrients, microorganisms, in vivo sensors, and tracers. In some embodiments, the agent (e.g., active substance), is a therapeutic, nutraceutical, prophylactic or diagnostic agent. The agent (e.g., active substance) may be entrapped within the polymer network or may be directly attached to one or more polymers in the polymer network through a chemical bond. In certain embodiments, the active substance is covalently bonded to one or more polymers of the polymer network. For example, in some embodiments, the active substance is bonded to a polymer through a carboxylic acid derivative. In some cases, the carboxylic acid derivative may form an ester bond with the active substance.

Agents can include, but are not limited to, any synthetic or naturally-occurring biologically active compound or composition of matter which, when administered to a subject (e.g., a human or nonhuman animal), induces a desired pharmacologic, immunogenic, and/or physiologic effect by local and/or systemic action. For example, useful or potentially useful within the context of certain embodiments are compounds or chemicals traditionally regarded as drugs, vaccines, and biopharmaceuticals. Certain such agents may include molecules such as proteins, peptides, hormones, nucleic acids, gene constructs, etc., for use in therapeutic, diagnostic, and/or enhancement areas, including, but not limited to medical or veterinary treatment, prevention, diagnosis, and/or mitigation of disease or illness (e.g., HMG co-A reductase inhibitors (statins) like an inhibitor (e.g., a nicotinamide adenine dinucleotide biosynthesis inhibitor like GMX-1778, FK866), rosuvastatin, nonsteroidal anti-inflammatory drugs like meloxicam, selective serotonin reuptake inhibitors like escitalopram, blood thinning agents like clopidogrel, steroids like prednisone, antipsychotics like aripiprazole and risperidone, analgesics like buprenorphine, antagonists like naloxone, montelukast, and memantine, cardiac glycosides like digoxin, alpha blockers like tamsulosin, cholesterol absorption inhibitors like ezetimibe, metabolites like colchicine, antihistamines like loratadine and cetirizine, opioids like loperamide, proton-pump inhibitors like omeprazole, anti(retro) viral agents like entecavir, dolutegravir, rilpivirine, and cabotegravir, antibiotics like doxycycline, ciprofloxacin, and azithromycin, anti-malarial agents, and synthroid/levothyroxine); substance abuse treatment (e.g., methadone and varenicline); family planning (e.g., hormonal contraception); performance enhancement (e.g., stimulants like caffeine); and nutrition and supplements (e.g., protein, folic acid, calcium, iodine, iron, zinc, thiamine, niacin, vitamin C, vitamin D, and other vitamin or mineral supplements).

In some embodiments, the agent (e.g., active substance) is a radiopaque material such as tungsten carbide or barium sulfate.

In some embodiments, the carrier may be present in combination with the therapeutic agent in the composition. In some embodiments, the therapeutic agent may be associated with the carrier by covalent or noncovalent interactions, and/or may be encapsulated by the carrier. In some embodiments, the one or more therapeutic agents in/are covalently bound to one or more materials of the carrier via a linker. In some embodiments, the one or more therapeutic agents is/are non-covalently bound to one or more materials of the carrier, for example by electrostatic interactions, hydrogen bonding, dipole interactions, and/or Van der Waals forces. In certain embodiments, the composition is constructed and arranged to release the therapeutic agent (e.g., active substance, pharmaceutical) from the carrier (e.g., polymer, polymer network). Such embodiments may be useful in the context of drug delivery. In other embodiments, the therapeutic agent (e.g., active substance, pharmaceutical) is permanently affixed to the composition. Such embodiments may be useful in molecular recognition and purification contexts. In certain embodiments, the therapeutic agent (e.g., active substance, pharmaceutical) is embedded within the composition. In some embodiments, the therapeutic agent (e.g., active substance, pharmaceutical) is associated with the composition (e.g., associated with one or more polymers, e.g., of the polymer network) via formation of a bond, such as an ionic bond, a covalent bond, a hydrogen bond, Van der Waals interactions, and the like. The covalent bond may be, for example, carbon-carbon, carbon-oxygen, oxygen-silicon, sulfur-sulfur, phosphorus-nitrogen, carbon-nitrogen, metal-oxygen, or other covalent bonds. The hydrogen bond may be, for example, between hydroxyl, amine, carboxyl, thiol, and/or similar functional groups

The compositions may be prepared using any method known in this art. These include, but are not limited to, spray drying, single emulsion solvent evaporation, double emulsion solvent evaporation, solvent extraction, phase separation, simple coacervation, complex coacervation, and other methods well known to those of ordinary skill in the art. The one or more therapeutic agents can be loaded into the composition via standard methods including, but not limited to, a single emulsion technique, powder mixing, direct addition, solvent loading, melt loading, physical blending, supercritical carbon dioxide assisted, and conjugation reactions such as ester linkages and amide linkages. The conditions used in preparing the composition may be altered to yield a composition (e.g., particles) of a desired size or property (e.g., hydrophobicity, hydrophilicity, external morphology, shape, etc.). The method of preparing the composition and the conditions (e.g., solvent, temperature, concentration, etc.) used may also depend on the agent and/or the composition of the carrier. Those of ordinary skill in the art would be capable of selecting suitable methods for producing compositions described herein, based on the teachings of this specification. For example, a single emulsion technique may comprise processing the carrier and/or one or more agents to form the composition, using at least one fluid into which the carrier and/or one or more agents can be dissolved, by e.g., dissolving, mixing, vortexing, homogenization, solvent evaporation (e.g., by rotary evaporation or ambient evaporation), filtration, and/or washing. Any of the non-limiting illustrative steps in the single emulsion technique may be carried out one, two, three, four, or more times. In some embodiments, the at least one fluid may comprise a buffer. The buffer may comprise e.g., a citrate buffer, an acetate buffer, a phosphate buffer, a sodium phosphate buffer, phosphate buffered saline, tris buffered saline, or a combination or permutation thereof. In some embodiments, the at least one fluid may comprise water or an aqueous solution. The water or aqueous solution may comprise e.g., a solution of sodium dodecyl sulfate in water, a solution of tween 20 in water, deionized water, resistivity 18.2 MΩ·cm water, or a combination or permutation thereof.

The method of making the composition may further comprise dispersion of the composition (e.g. particles comprising a polymer and an agent, e.g., after washing) in at least one fluid (e.g., a buffer, deionized water).

The method of making the composition may further comprise lowering the temperature of the composition to a temperature less than or equal to 20° C., less than or equal to 10° C., less than or equal to 0° C., less than or equal to −10° C., less than or equal to −20° C., less than or equal to −30° C., less than or equal to −40° C., less than or equal to −50° C., less than or equal to −60° C., less than or equal to −70° C., less than or equal to −80° C., less than or equal to −90C, or less than or equal to −100° C. In some embodiments, the method may comprise lowering the temperature of the composition to a temperature greater than or equal to −100° C., greater than or equal to 90° C., greater than or equal to −80° C., or greater than or equal to −70° C. Combinations of the above referenced ranges are also possible. For example, in some embodiments, the method of making the composition may comprise lowering the temperature of the composition to a temperature of less than or equal to −80° C. and greater than or equal to −100° C.

In some embodiments, the method may comprise lyophilizing the composition for at least one hour, at least two hours, at least four hours, at least eight hours, at least 16 hours, at least 24 hours, at least 36 hours, at least 48 hours, at least 72 hours, at least 100 hours, or at least 120 hours. In some embodiments, the method may comprise lyophilizing the composition for at most hundred and 20 hours, at most 100 hours, and most 72 hours, and most 48 hours, at most 36 hours, at most 24 hours, and most 16 hours, at most eight hours, at most four hours, or at least two hours. Combinations of the above-referenced ranges are also possible. For example, in some embodiments, the composition may be lyophilized for at least 24 hours and at most hundred and 20 hours.

In an exemplary embodiment, the particles are formed using a single emulsion technique involving dissolving PLGA in chloroform and dissolving the agent in chloroform and methanol, and then mixing polymer and drug solutions together and adding to aqueous polyvinyl alcohol solution, followed by vortexing or homogenizing, diluting, evaporating the solvent (e.g., under reduced pressure), then filtering the microparticle dispersion, then washing the microparticles, dispersing in deionized water, freezing the microparticles to −80° C., and lyophilizing the particles.

In some embodiments, the particles are formed such that the amount of the therapeutic protein that is in crystal form is minimized. In some embodiments, the particles are formed such that there are no detectable crystals of the therapeutic protein in the composition of particles. Methods of assessing the amount and/or presence of the therapeutic in crystal form in the composition of particles will be evident to one of skill in the art, such as by scanning electron microscopy (SEM).

In some embodiments, the composition (e.g., pharmaceutical composition) is loaded (e.g., during and/or after formation of the polymer, e.g., polymer network, of the composition) with an active substance such as one or more therapeutic agents. In other embodiments, the composition is loaded with one or more therapeutic agents after it is already retained at a location internal to a subject, such as a gastric cavity. In some embodiments, a composition is configured to maintain stability of one or more therapeutic agents in a hostile physiological environment (e.g., the gastric environment) for an extended duration. In further embodiments, the composition is configured to control release of one or more therapeutic agents e.g., with low to no potential for burst release. In some embodiments, the composition is pre-loaded and/or loaded with a combination of active substances. For example, in certain embodiments, the structure comprises one or more, two or more, three or more, or four or more active substances.

Therapeutic agents (e.g., active substances) that contain a carboxylic acid group may be directly incorporated into a carrier (e.g., polymer, polymeric microparticle, polymer network) that contains ester and hydroxyl groups without further modification. Therapeutic agents (e.g., active substances) containing an alcohol may first be derivatized as a succinic or fumaric acid monoester and then incorporated into the carrier (e.g., polymer, polymeric microparticle, polymer network). Active substances that contain a thiol may be incorporated into an olefin or acetylene-containing carrier (e.g., polymer(s)) through a sulfur-ene (e.g., thiol-ene) reaction. In other embodiments, the one or more agents are non-covalently associated with the carrier (e.g., polymer, polymeric microparticle, polymer network, nanoparticles) (e.g., dispersed or encapsulated within the carrier, e.g., polymeric microparticle, polymer network, nanoparticles). In some such embodiments, the active substance may be dispersed or encapsulated within the carrier by hydrophilic and/or hydrophobic forces.

Those skilled in the art given the guidance and teaching of this specification would be capable of determining suitable methods for tuning the mechanical properties of the composition (e.g., pharmaceutical composition) by, for example, varying the chemical composition of the pharmaceutically-acceptable carrier (e.g., varying the chemical composition of a polymeric microparticle, e.g., by varying the molar ratios of lactide to glycolide in PLGA), varying the number average molecular weight of a polymeric carrier, varying the weight average molecular weight of a polymeric carrier, varying the molar ratios of monomeric and/or polymeric units, varying cross-linking density, varying the concentration of cross-linking agents used in the formation of the polymer, varying the crystallinity of the polymer (e.g., by varying the ratio of crystalline and amorphous regions in the polymer) and/or the use of additional or alternative materials.

Release of the one or more therapeutic agents can then be accomplished through methods including, but not limited to, dissolution of the composition comprising a carrier comprising a polymeric matrix material (e.g., polymeric microparticles, nanoparticles), degradation of the carrier (e.g., matrix material), swelling of the carrier (e.g., matrix material), diffusion of an agent, hydrolysis, and chemical or enzymatic cleavage of conjugating bonds. In some embodiments, the one or more agents (e.g., active substance) is/are covalently bound to one or more materials (e.g., polymers) of the carrier (e.g., polymer, e.g., polymer network) (e.g., and is released while the composition resides at a location internal to a subject).

In some embodiments, the therapeutic agent (e.g., the active substance) may be released from the composition. In certain embodiments, the therapeutic agent (e.g., active substance) is released by diffusion out of the composition. In some embodiments, the therapeutic agent (e.g., active substance) is released by degradation of the composition (e.g., biodegradation, enzymatic degradation, hydrolysis). In some embodiments, the therapeutic agent (e.g., active substance) is released from the composition at a particular rate. In some embodiments, the therapeutic agent (e.g., the NAD biosynthesis inhibitor) is released from the carrier with a sustained release profile.

Those skilled in the art would understand that the rate of release may be dependent, in some embodiments, on the solubility of the active substance in the medium in which the composition is exposed, such as a physiological fluid such as gastric fluid or cerebrospinal fluid. The ranges and description included related to the release and/or rate of release of the active substance is generally in reference to hydrophilic, hydrophobic, and/or lipophilic active substances in simulated cerebrospinal fluid (e.g., as defined in the United States Pharmacopeia (USP)). Simulated cerebrospinal fluids are known in the art and those skilled in the art would be capable of selecting suitable simulated cerebrospinal fluids based on the teachings of this specification.

In some embodiments, between 0.05 wt % to 99 wt % of the therapeutic agent initially contained in a composition is released (e.g., in vivo) between 24 hours and 1 year. In some embodiments, between about 0.05 wt % and about 99.0 wt % of the therapeutic agent is released (e.g., in vivo) from the composition after a certain amount of time. In some embodiments, at least about 0.05 wt %, at least about 0.1 wt %, at least about 0.5 wt %, at least about 1 wt %, at least about 5 wt %, at least about 10 wt %, at least about 20 wt %, at least about 50 wt %, at least about 75 wt %, at least about 90 wt %, at least about 95 wt %, or at least about 98 wt % of the therapeutic agent associated with the composition is released from the composition (e.g., in vivo) within about 24 hours, within 36 hours, within 72 hours, within 96 hours, or within 192 hours. In certain embodiments, at least about 0.05 wt %, at least about 0.1 wt %, at least about 0.5 wt %, at least about 1 wt %, at least about 5 wt %, at least about 10 wt %, at least about 20 wt %, at least about 50 wt %, at least about 75 wt %, at least about 90 wt %, at least about 95 wt %, or at least about 98 wt % of the therapeutic agent associated with the composition is released from the composition (e.g., in vivo) within 1 day, within 5 days, within 30 days, within 60 days, within 120 days, or within 365 days. For example, in some cases, at least about 90 wt % of the therapeutic agent associated with the composition is released from the composition (e.g., in vivo) within 120 days.

In some embodiments, the therapeutic agent (e.g., active substance) is released from the carrier (e.g., loadable polymeric material, polymeric microparticle) at a particular initial average rate as determined over the first 24 hours of release (the “initial rate”) (e.g., release of the active substance at the desired location internally of the subject, such as an internal cavity). In certain embodiments, the therapeutic agent (e.g., active substance) is released at an average rate of at least about 1%, at least about 2%, at least about 5%, least about 10%, at least about 20%, at least about 30%, least about 50%, at least about 75%, at least about 80%, at least about 90%, at least about 95%, or at least about 98% of the initial average rate over a 24 hour period after the first 24 hours of release. In some embodiments, the therapeutic agent (e.g., active substance) is released at an average rate of less than or equal to about 99%, less than or equal to about 98%, less than or equal to about 95%, less than or equal to about 90%, less than or equal to about 80%, less than or equal to about 75%, less than or equal to about 50%, less than or equal to about %, less than or equal to about 30%, less than or equal to about 20%, less than or equal to about 10%, less than or equal to about 5%, or less than or equal to about 2% of the initial average rate over a 24 hour period after the first 24 hours of release. Any and all closed ranges that have endpoints within any of the above referenced ranges are also possible (e.g., between about 1% and about 99%, between about 1% and about 98%, between about 2% and about 95%, between about 10% and about 30%, between about 20% and about 50%, between about 30% and about 80%, between about 50% and about 99%). Other ranges are also possible.

The therapeutic agent (e.g., active substance) may be released at an average rate over at least one selected continuous 24 hour period at a rate of between about 1% and about 99% of the initial rate between 48 hours and about 1 year (e.g., between 48 hours and 1 week, between 3 days and 1 month, between 1 week and 1 month, between 1 month and 6 months, between 3 months and 1 year, between 6 months and 2 years) after the initial release.

For example, in some cases, the therapeutic agent (e.g., active substance) may be released at a rate of between about 1% and about 99% of the initial rate on the second day of release, the third day of release, the fourth day of release, the fifth day of release, the sixth day of release, and/or the seventh day of release. In certain embodiments, burst release of therapeutic agent from the composition is generally avoided. In an illustrative embodiment, in which at least about 0.05 wt % of the therapeutic agent (e.g., active substance) is released from the composition within 24 hours, between about 0.05 wt % and about 99 wt % is released during the first day of release (e.g., at the location internally of the subject), and between about 0.05 wt % and about 99 wt % is released during the second day of release. Those skilled in the art would understand that the therapeutic agent (e.g., active substance) may be further released in similar amounts during a third day, a fourth day, a fifth day, etc. depending on the properties of the composition and/or the therapeutic agent.

In certain embodiments, the therapeutic agent (e.g., active substance) may be released with a pulse release profile. For example, in some embodiments, the therapeutic agent (e.g., active substance) may be released on the first day after administration and during another 24 hour period such as starting during the third day, the fourth day, or the fifth day, but is not substantially released on other days. Those skilled in the art would understand that other days and/or combinations of pulsing and continuous release are also possible.

The therapeutic agent (e.g., active substance) may be released at a relatively constant average rate (e.g., a substantially zero-order average release rate) over a time period of at least about 24 hours. In certain embodiments, the therapeutic agent (e.g., active substance) is released at a first-order release rate (e.g., the rate of release of the active substance is generally proportional to the concentration of the active substance) of a time period of at least about 24 hours.

In some embodiments, at least a portion of the therapeutic agent (e.g., active substance) loaded into the composition is released continuously (e.g., at varying rates) over the residence time period of the composition.

In some embodiments, the composition (e.g., pharmaceutical composition) may be stable under ambient conditions (e.g., room temperature, atmospheric pressure and relative humidity) and/or physiological conditions (e.g., at or about 37° C., in physiologic fluids) for at least about 1 day, at least about 3 days, at least about 7 days, at least about 2 weeks, at least about 1 month, at least about 2 months, at least about 6 months, at least about 1 year, or at least about 2 years. In certain embodiments, the composition may be stable for less than or equal to about 3 years, less than or equal to about 2 years, less than or equal to about 1 year, less than or equal to about 1 month, less than or equal to about 1 week, or less than or equal to about 3 days. Any and all closed ranges that have endpoints within any of the above-referenced ranged are also possible (e.g., between about 24 hours and about 3 years, between about 1 week and 1 year, between about 1 year and 3 years). Other ranges are also possible.

### Pharmaceutical Compositions

Any of the compositions described herein may be mixed with a pharmaceutically acceptable excipient to form a pharmaceutical composition for administration to a subject. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins. Ed. K. E. Hoover. Examples of such buffers and diluents are distilled water, physiological saline, Ringer's solution, dextrose solution, and Hank's solution. The same diluents may be used to reconstitute a lyophilized recombinant protein of interest. In addition, the pharmaceutical composition may also include other medicinal agents, pharmaceutical agents, carriers, adjuvants, nontoxic, non-therapeutic, non-immunogenic stabilizers, etc. Effective amounts of such diluent or carrier are amounts which are effective to obtain a pharmaceutically acceptable formulation in terms of solubility of components, biological activity, etc. In some embodiments the compositions provided herein are sterile.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

### Cancer Therapeutics

Any of the methods described herein may further comprise administering one or more cancer therapeutics to the subject. In some embodiments, the methods involve locally administering a cancer therapeutic to the subject. In some embodiments, the methods involve locally administering a cancer therapeutic to the subject, if it has been determined that the cancer has a mutant genotype, for example using the methods described herein.

In some embodiments, the subject is administered one or more additional cancer therapeutics at time following locally administering a metabolic inhibitor, such as any of the compositions described herein. In some embodiments, the subject is systemically administered one or more additional cancer therapeutics. In some embodiments, one or more additional cancer therapeutic is administered systemically to the subject at least one week, 2 weeks, 3 weeks, 4 weeks, 5 weeks, 6 weeks 7 weeks, 8 weeks, or longer following local administration the metabolic inhibitor.

The term “cancer therapeutic” refers to agents that are useful in treating cancer and generally include agents that reduce neovascularization, prevent further neovascularization, reduce tumor size, prevent further tumor growth, and/or destroy or inhibit cancerous cell growth or activity. Examples of chemotherapeutic agents include, without limitation, azacitidine, azathioprine, bleomycin, carboplatin, capecitabine, cisplatin, chlorambucil, cyclophosphamide, cytarabine, daunorubicin, docetaxel, doxifluridine, doxorubicin, epirubicin, etoposide, fluorouracil, gemcitabine, hydroxyurea, idarubicin, mechlorethamine, mercaptopurine, methotrexate, mitoxantrone, oxaliplatin, paclitaxel, pemetrexed, temozolomide, teniposide, thioguanine, valrubicin, vinblastine, vincristine, vindesine, and vinorelbine. Additional examples of antineoplastic agents include adriamycin, actinomycin, mitomycin, carmustine (BCNU), methyl-CCNU, interferons, phenesterine, taxol and derivatives thereof, taxotere and derivatives thereof, tamoxifen, piposulfan, and flutamide, and derivatives thereof. Additional cancer therapeutics suitable for use in the methods described herein will be recognized by one of ordinary skill in the art.

In some embodiments, the subject is systemically administered a NAMPT inhibitor (e.g., at a dose demonstrated to have reduced toxicity) at a time following local administration of a NAMPT inhibitor during the course of a surgical resectioning procedure.

### Kits

The present disclosure also provides kits for use in rapidly determining the genotype of cells in a sample obtained from a subject. Such kits can comprise agents for performing an assay, e.g., a polymerase chain reaction-based assay, for rapidly determining the genotype of cells of a sample obtained from a subject. In some embodiments, the kits may also contain a therapeutic agent (e.g., a metabolic inhibitor) for administering to the subject if a specific genotype is determined.

In some embodiments, the kit comprises a set of primers for amplifying a target nucleic acid, a probe for detecting the presence of an amplified target nucleic acid, and/or a peptide nucleic acid (PNA) blocker that clamps (blocks) the wildtype nucleotide sequence such that a wildtype sequence, if present in cells of the sample, is not amplified.

A kit, as described herein, may be for detection of a specific genotype. In some embodiments, a kit is for detection of mutations at a specific codon of a gene. In some embodiments, a kit is for detection of mutations at a specific codon of more than one genes, for example a panel of genes.

In some embodiments, the kit is for detecting a mutation in IDH1. In some embodiments, the kit includes a set of primers for amplification of a mutant allele of IDH1 (a IDH1 variant), if present in the sample. In some embodiments, the kit includes more than one sets of primers for amplification of more than one mutant allele (e.g., R132H, R132C, R132G, R132S, R132L) of IDH1 (a IDH1 variant), if present in the sample. In some embodiments, the kit also includes a probe for detection of a mutant allele of IDH1 (a IDH1 variant). In some embodiments, the kit also includes a PNA blocker that clamps (blocks) the wildtype nucleotide sequence and prevents amplification of a wildtype allele.

In some embodiments, the kit is for detection of a IDH1 R132H variant. In some embodiments, the kit includes a forward primer having a nucleotide sequence set forth by SEQ ID NO: 2 and a reverse primer having a nucleotide sequence set forth by SEQ ID NO: 18. In some embodiments, the kit includes a probe having a nucleotide sequence set forth by SEQ ID NO: 9. In some embodiments, the kit includes a PNA blocker having a nucleotide sequence set forth by SEQ ID NO: 24.

In some embodiments, the kit is for detection of a IDH1 R132C variant. In some embodiments, the kit includes a forward primer having a nucleotide sequence set forth by SEQ ID NO: 3 and a reverse primer having a nucleotide sequence set forth by SEQ ID NO: 18. In some embodiments, the kit includes a probe having a nucleotide sequence set forth by SEQ ID NO: 10. In some embodiments, the kit includes a PNA blocker having a nucleotide sequence set forth by SEQ ID NO: 24.

In some embodiments, the kit is for detection of a IDH1 R132G variant. In some embodiments, the kit includes a forward primer having a nucleotide sequence set forth by SEQ ID NO: 3 and a reverse primer having a nucleotide sequence set forth by SEQ ID NO: 18. In some embodiments, the kit includes a probe having a nucleotide sequence set forth by SEQ ID NO: 11. In some embodiments, the kit includes a PNA blocker having a nucleotide sequence set forth by SEQ ID NO: 24.

In some embodiments, the kit is for detection of a IDH1 R132S variant. In some embodiments, the kit includes a forward primer having a nucleotide sequence set forth by SEQ ID NO: 3 and a reverse primer having a nucleotide sequence set forth by SEQ ID NO: 18. In some embodiments, the kit includes a probe having a nucleotide sequence set forth by SEQ ID NO: 12. In some embodiments, the kit includes a PNA blocker having a nucleotide sequence set forth by SEQ ID NO: 24.

In some embodiments, the kit is for detection of a IDH1 R132L variant. In some embodiments, the kit includes a forward primer having a nucleotide sequence set forth by SEQ ID NO: 3 and a reverse primer having a nucleotide sequence set forth by SEQ ID NO: 18. In some embodiments, the kit includes a probe having a nucleotide sequence set forth by SEQ ID NO: 13. In some embodiments, the kit includes a PNA blocker having a nucleotide sequence set forth by SEQ ID NO: 24.

In some embodiments, the kit is for detecting a mutation in the TERT promoter. In some embodiments, the kit includes a set of primers for amplification of a mutant allele of the TERT promoter (a TERT variant), if present in the sample. In some embodiments, the kit includes more than one sets of primers for amplification of more than one mutant allele (e.g., C228T, C250T) of the TERT promoter (a TERT variant), if present in the sample. In some embodiments, the kit also includes a probe for detection of a mutant allele of the TERT promoter (a TERT variant). In some embodiments, the kit also includes a PNA blocker that clamps (blocks) the wildtype nucleotide sequence and prevents amplification of a wildtype allele.

In some embodiments, the kit is for detection of a TERT C228T variant. In some embodiments, the kit includes a forward primer having a nucleotide sequence set forth by SEQ ID NO: 1 and a reverse primer having a nucleotide sequence set forth by SEQ ID NO: 17. In some embodiments, the kit includes a probe having a nucleotide sequence set forth by SEQ ID NO: 7. In some embodiments, the kit includes a PNA blocker having a nucleotide sequence set forth by SEQ ID NO: 22.

In some embodiments, the kit is for detection of a TERT C250T variant. In some embodiments, the kit includes a forward primer having a nucleotide sequence set forth by SEQ ID NO: 1 and a reverse primer having a nucleotide sequence set forth by SEQ ID NO: 17. In some embodiments, the kit includes a probe having a nucleotide sequence set forth by SEQ ID NO: 8. In some embodiments, the kit includes a PNA blocker having a nucleotide sequence set forth by SEQ ID NO: 23.

In some embodiments, the kit is for detecting a mutation in H3F3A. In some embodiments, the kit includes a set of primers for amplification of a mutant allele of H3F3A (a H3F3A variant), if present in the sample. In some embodiments, the kit includes more than one sets of primers for amplification of more than one mutant allele of the H3F3A promoter (a H3F3A variant), if present in the sample. In some embodiments, the kit also includes a probe for detection of a mutant allele of H3F3A (a H3F3A variant). In some embodiments, the kit also includes a PNA blocker that clamps (blocks) the wildtype nucleotide sequence and prevents amplification of a wildtype allele.

In some embodiments, the kit is for detection of a H3F3A K27M variant. In some embodiments, the kit includes a forward primer having a nucleotide sequence set forth by SEQ ID NO: 4 and a reverse primer having a nucleotide sequence set forth by SEQ ID NO: 19. In some embodiments, the kit includes a probe having a nucleotide sequence set forth by SEQ ID NO: 14. In some embodiments, the kit includes a PNA blocker having a nucleotide sequence set forth by SEQ ID NO: 25.

In some embodiments, the kit is for detecting a mutation in the BRAF. In some embodiments, the kit includes a set of primers for amplification of a mutant allele of BRAF (a BRAF variant), if present in the sample. In some embodiments, the kit includes more than one sets of primers for amplification of more than one mutant allele of BRAF (a BRAF variant), if present in the sample. In some embodiments, the kit also includes a probe for detection of a mutant allele of BRAF (a BRAF variant). In some embodiments, the kit also includes a PNA blocker that clamps (blocks) the wildtype nucleotide sequence and prevents amplification of a wildtype allele.

In some embodiments, the kit is for detection of a BRAF V600E variant. In some embodiments, the kit includes a forward primer having a nucleotide sequence set forth by SEQ ID NO: 5 and a reverse primer having a nucleotide sequence set forth by SEQ ID NO: 20. In some embodiments, the kit includes a probe having a nucleotide sequence set forth by SEQ ID NO: 15. In some embodiments, the kit includes a PNA blocker having a nucleotide sequence set forth by SEQ ID NO: 26.

The kit may further comprise a support member for performing the assay. In some embodiments, the support member is a 96-well plate, such as a 96-well PCR plate.

The kit can also comprise one or more buffers as described herein but not limited to a PCR buffer. In general, PCR buffers may include Tris-HCl, KCl, and MgCl₂. In some embodiments, the kit may also comprise one or more additional components for performing the assay. In some embodiments, the kit may also comprise deoxynucleotide triphosphate solution mix (dNTPs). In some embodiments, the kit may also comprise polymerase, such as a DNA polymerase. In general, DNA polymerases suitable for use in the assay methods described herein are known in the art and may be commercially available.

In some embodiments, the kit may also comprise one or more components for extracting nucleic acid from the sample.

In some embodiments, the kit can comprise instructions for use in accordance with any of the methods described herein. The included instructions can comprise a description of how to use the components contained in the kit for measuring the level of proteins of a biomarker set in a biological sample collected from a subject, such as a human patient.

The instructions relating to the use of the kit generally include information as to the amount of each component and suitable conditions for performing the assay methods described herein. The components in the kits may be in unit doses, bulk packages (e.g., multi-dose packages), or sub-unit doses. Instructions supplied in the kits of the present disclosure are typically written instructions on a label or package insert (e.g., a paper sheet included in the kit), but machine-readable instructions (e.g., instructions carried on a magnetic or optical storage disk) are also acceptable.

The label or package insert indicates that the kit is used for determining the genotype of cells in the sample. Instructions may be provided for practicing any of the methods described herein.

The kits of this present disclosure are in suitable packaging. Suitable packaging includes, but is not limited to, vials, bottles, jars, flexible packaging (e.g., sealed Mylar or plastic bags), and the like. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle).

Kits may optionally provide additional components such as interpretive information, such as a control and/or standard or reference sample. In some embodiments, the kit may comprise one or more components for amplifying a control gene, such as a primer set for amplifying a control gene in the sample. In some embodiments, the control gene is GAPDH. In some embodiments, the kit includes a forward primer having a nucleotide sequence set forth by SEQ ID NO: 6 and a reverse primer having a nucleotide sequence set forth by SEQ ID NO: 21. In some embodiments, the kit also includes a probe for detection of the control gene. In some embodiments, the probe has a nucleotide sequence set forth by SEQ ID NO: 16.

In some embodiments, the kit also includes a PNA blocker that clamps (blocks) the wildtype nucleotide sequence and prevents amplification of a wildtype allele.

Normally, the kit comprises a container and a label or package insert(s) on or associated with the container. In some embodiments, the present disclosure provides articles of manufacture comprising contents of the kits described above.

Unless otherwise defined herein, scientific and technical terms used in connection with the present disclosure shall have the meanings that are commonly understood by those of ordinary skill in the art. Further, unless required by context, singular terms shall include pluralities and plural terms shall include the singular. The methods and techniques of the present disclosure are generally performed according to conventional methods well-known in the art. Generally, nomenclatures used in connection with, and techniques of biochemistry, enzymology, molecular and cellular biology, microbiology, genetics and protein and nucleic acid chemistry and hybridization described herein are those well-known and commonly used in the art. The methods and techniques of the present disclosure are generally performed according to conventional methods known in the art and as described in various general and more specific references that are cited and discussed throughout the present specification unless otherwise indicated.

### Example

Aggressive neurosurgical resectioning to achieve sustained local control of a cancer is necessary for prolonging survival in patients with infiltrative gliomas. Nevertheless, cancer recurrences commonly arise from the tumor-infiltrated normal brain tissue margin. Glioma characterized by IDH1 mutations have been found to be sensitive to metabolism-altering agents, such as inhibitors of NAD biosynthesis. Described herein is the development of a rapid diagnostic method coupled with a sustained release drug delivery system enabling intraoperative genotype-directed modulation of the metabolic microenvironment to improve local control of IDH1 mutant gliomas.

Retrospective Analysis of Diffuse Astrocytomes with Local Failure

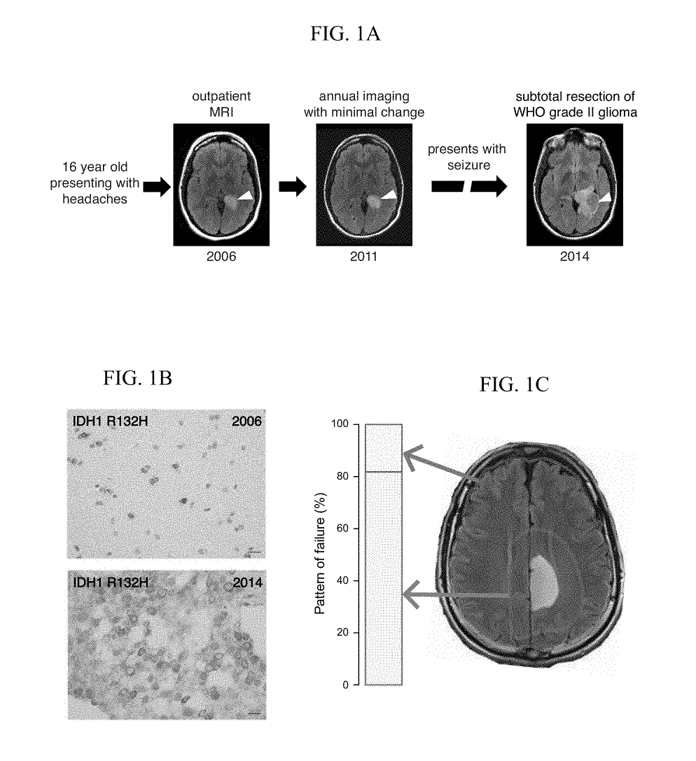

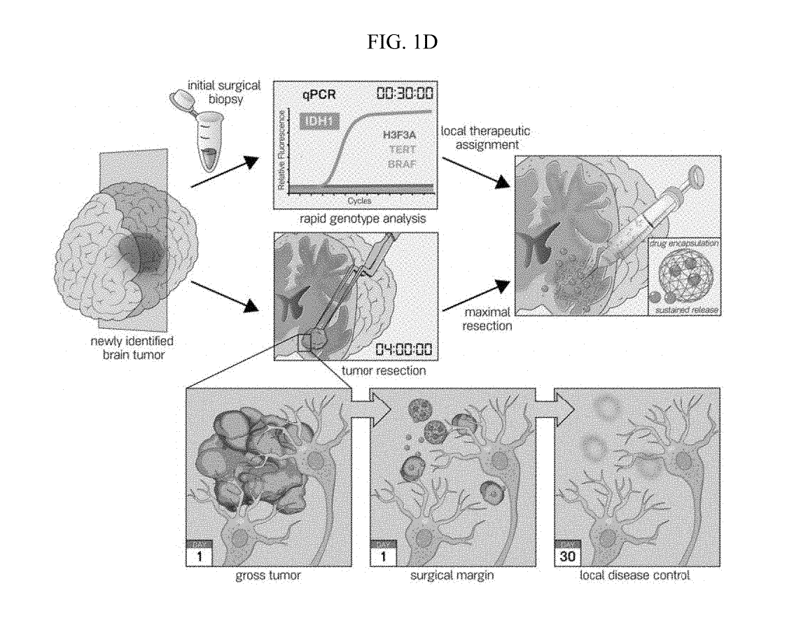

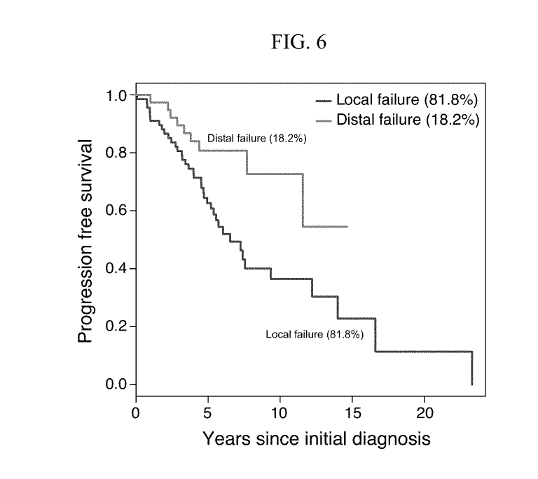

The most common pattern of progression in diffuse astrocytomas is local failure (FIGS. 1A-1B). A cohort of 130 patients who underwent resection of an IDH1/2 mutant astrocytoma between 1988 and 2014 with a median follow up of 4.5 years (1 month-23.3 years) were retrospectively analyzed. A median progression free survival of 4.7 years was noted(FIG. 6). Importantly, it was found that 82% of these cases were characterized by progressive disease within 2 cm of the initial tumor margin (FIG. 1C). Interestingly, patients experiencing local failure had a lower overall survival in comparison with those experiencing distal failure. To directly address this aspect of the natural history of diffuse astrocytomas a paradigm was developed by which genotype-directed therapeutics are applied along the surgical margin at the time of resection (FIG. 1D). The development of this paradigm requires (1) the ability to categorize the molecular phenotype of the glioma specimen intraoperatively and (2) a mode for providing local, sustained delivery of therapeutic compounds.

### NAMPT Inhibitor Serum Levels and Toxicities Following Enteral Administration

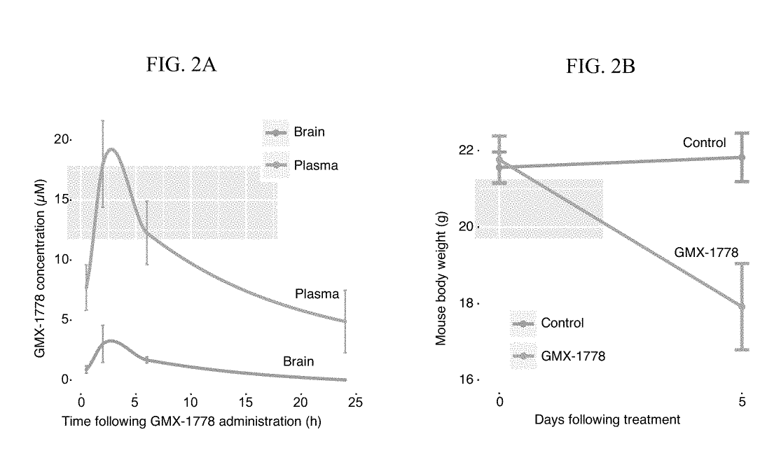

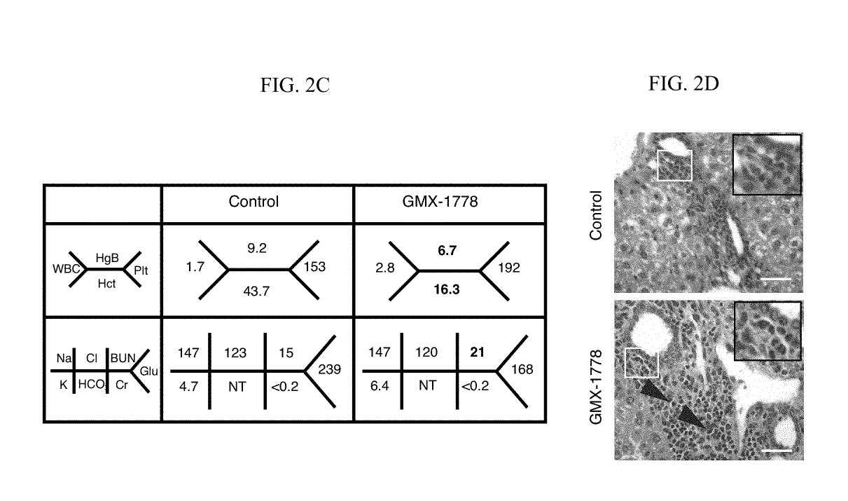

Prior studies have indicated that direct inhibitors of mutant IDH1 enzyme in immortalized human glioma cell lines may confer an undesirable increase in cell viability. Instead, it has been noted that IDH1/2 mutated glioma cells are susceptible to NAD+ depletion given their dependency on a salvage production pathway via NAMPT¹⁷. GMX-1778, a NAMPT inhibitor with partial blood-brain barrier permeability, has been previously demonstrated to increase the overall survival of orthotopic mouse models of IDH1 mutant glioma. It was found that these previously reported doses that have been demonstrated to achieve this therapeutic effect reach peak concentration of 18.0±3.6 μM in the plasma and 3.0±1.5 μM in the brain within 2 hours of enteral gavage of 250 mg/kg in mice. It was also noted that within 24 hours, GMX-1778 was no longer detectable in brain, suggesting daily dosing would be required to maintain a therapeutic intracerebral concentration (FIG. 2A). Previous human trials of NAMPTi were halted for concerns of retinal toxicity and thrombocytopenia¹⁸. Importantly, these toxicities could not be rescued by co-administration of nicotinic acid, a precursor for NAD+ production via an NAMPT-independent pathway. Here, weight loss following 5 day oral administration of GMX-1778 was noted (FIG. 2B, 17.9±1.1 g, n=5 vs 21.8±0.6 g, n=9 control dextrose treated animals, p<0.05). GMX-1778 treated animals were noted to have anemia (hemoglobin 6.7±0.8 g/dL, n=4 vs 9.2±0.5 g/dL, n=5, p<0.05) and uremia (20.5±1.9 mg/dL, n=4, vs 15±0.5 mg/dL, n=4, p<0.05) (FIG. 2C). Furthermore, it was noted that liver from mice treated with GMX-1778 were characterized by eosinophilic cholangitis (FIG. 2D).

### NAMPT Inhibitor Formulation Development and Characterization for Local Delivery

These systemic toxicities are especially problematic for patients with IDH1 mutant gliomas, given their typically younger age at presentation and anticipated long progression-free survival. Therefore, it was sought to develop a method for local delivery of NAMPTi to the surgical margin. For initial formulations, two chemically-dissimilar agents, GMX-1778, which is partially blood-brain barrier penetrant, and FK866, which does not measurably accumulate in the brain parenchyma when given systemically, were selected. In practice, an ideal formulation would provide sustained release of inhibitor over a clinically-relevant time-frame, in the weeks following initial diagnostic surgical resection until the initiation of adjuvant therapy. Given these design parameters, it was opted to formulate NAMPT inhibitor in microparticles made of poly(lactic-co-glycolic acid) (PLGA), a well-established, safe and biodegradable co-polymer.

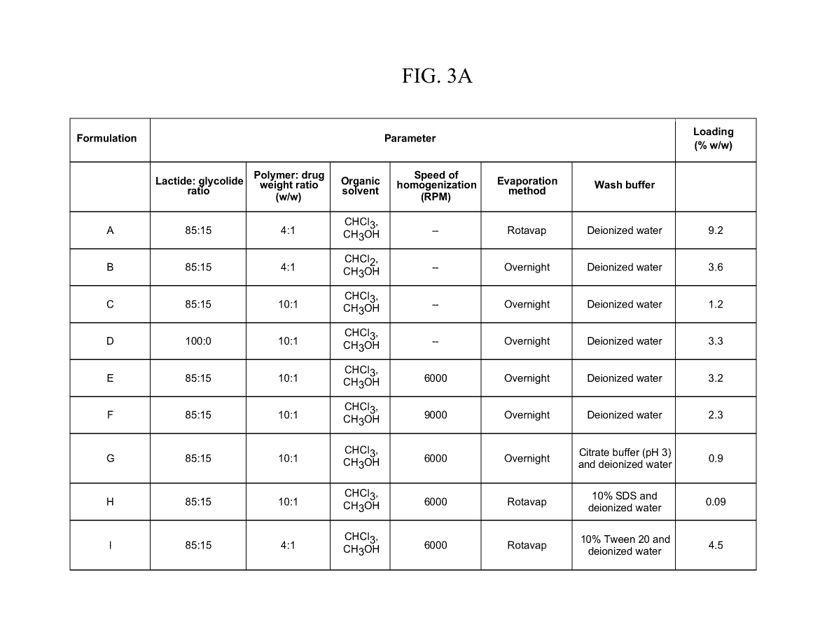

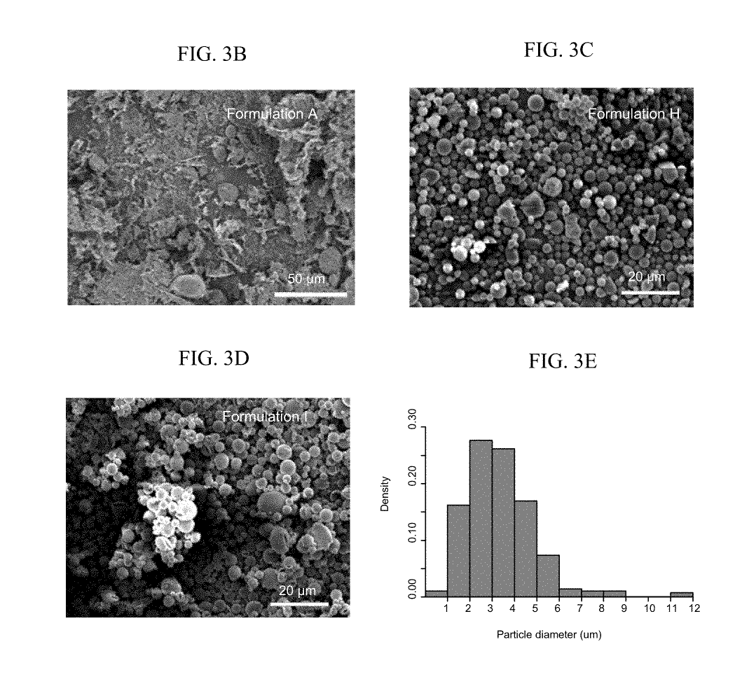

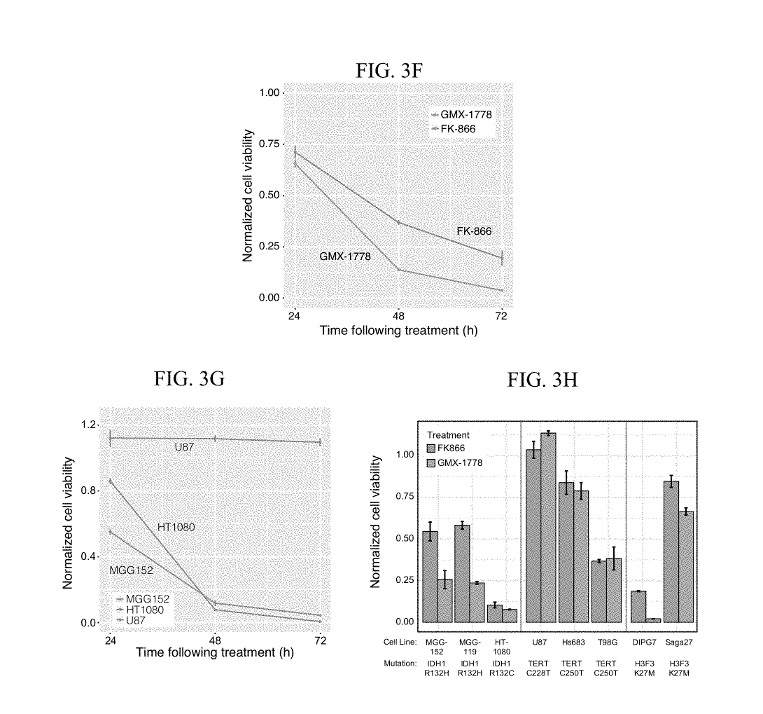

Different formulations of GMX-1778 were synthesized, varying parameters such as polymer: drug ratio, lactide: glycolide ratio, method of solvent evaporation and method of mixing (FIG. 3A). Eight (Formulations A-H) were evaluated for drug loading and crystal formation by HPLC and SEM respectively. In some cases, microparticles prepared by rapid solvent evaporation were noted to have significant numbers of unbound drug crystals (e.g., Formulation A, FIG. 3B). These crystals could be removed from the formulation by washing the particles with an ionic surfactant in which drug had high solubility, but this process markedly reduced drug loading (<0.1% w/w, Formulation H, FIG. 3C). Conversely, by washing the particles with non-ionic surfactant, a drug loading of 4.5% w/w was achieved, in a preparation otherwise free of drug crystals (Formulation I, FIG. 3D). In this formulation, the particle size distribution was unimodal with peak size measuring between 3-4 μm (FIG. 3E).

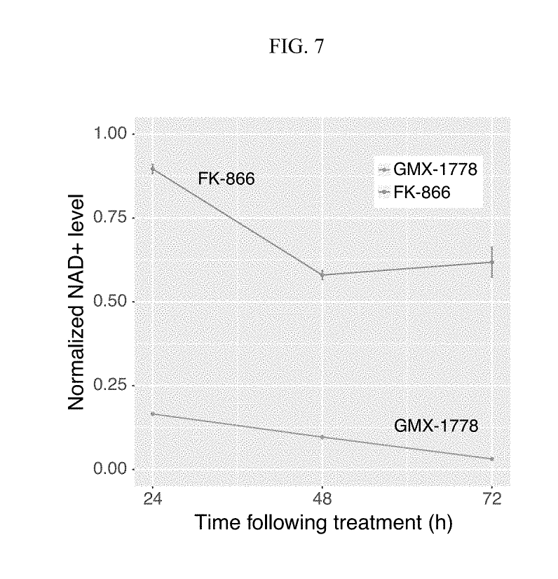

The in vitro bioactivity of GMX-1778 microparticles from Formulation I was tested. A time-dependent decrease in cell viability was observed when microparticles were co-incubated with IDH1-mutant MGG152 cells, resulting in a 34.5±1.7% decrease in viability at 24 hours and 96.3±0.2% decrease at 72 hours (FIG. 3F, n=3). This effect on cell viability correlated with an on-target pharmacodynamic effect of decreased NAD+ levels of 83±1% at 24 hours and 97±0.1% at 72 hours (FIG. 7). Similar decreases in cell viability and NAD+ level were noted with a microparticle formulation using the alternative inhibitor, FK-866 (FIG. 3F and FIG. 7).

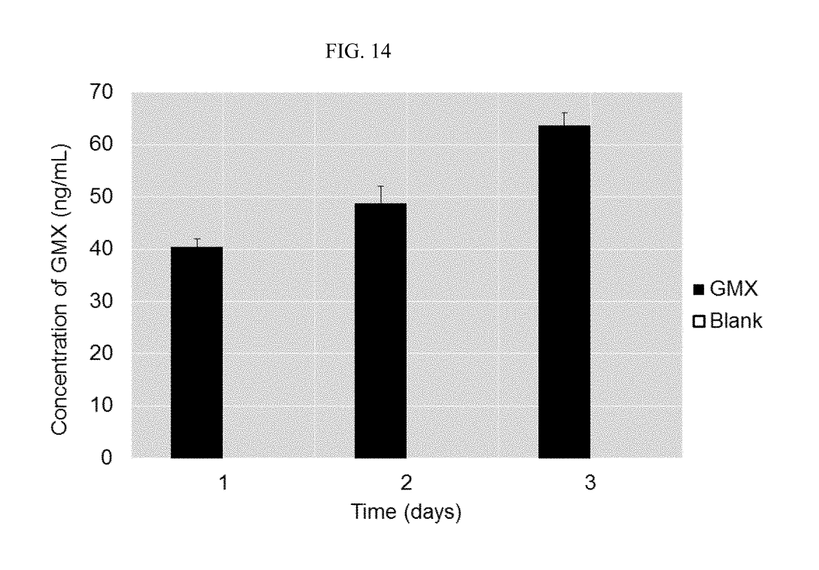

Analysis of drug concentration in the media revealed a time-dependent increase in GMX-1778 of 40.4±2.3 nM at 24 hours and 63.8±3.7 nM at 72 hours (FIG. 14). Furthermore, the effect on cell viability was observed in glioma cells that harbor the IDH1 mutation, and viability of other glioma cells characterized by mutations in the TERT promoter or H3F3A histone gene were varied (FIGS. 3G-3H). In fact, a subtle increase in cell viability was noted when glioblastoma cells (TERT promoter mutant, IDH1 wild-type U87) were co-incubated with GMX-1778 microparticles (109.6±3.4% at 72 hours).

### Rapid Mutation Tissue Genotyping

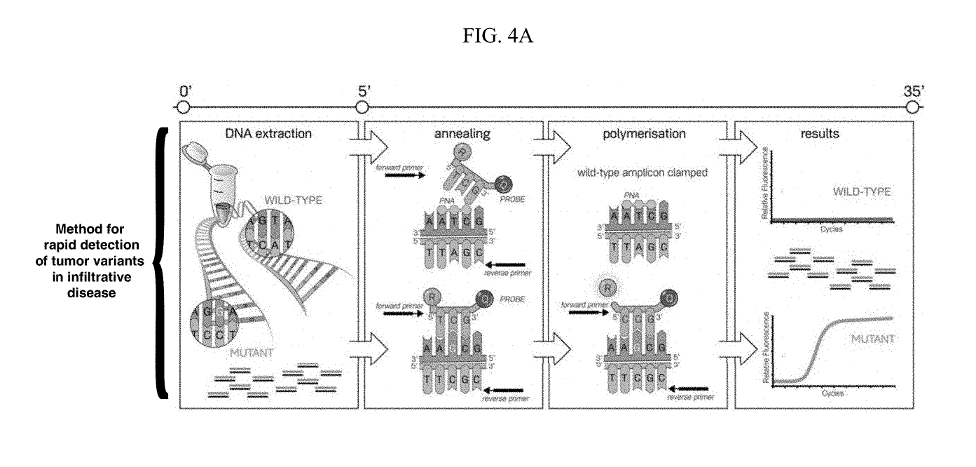

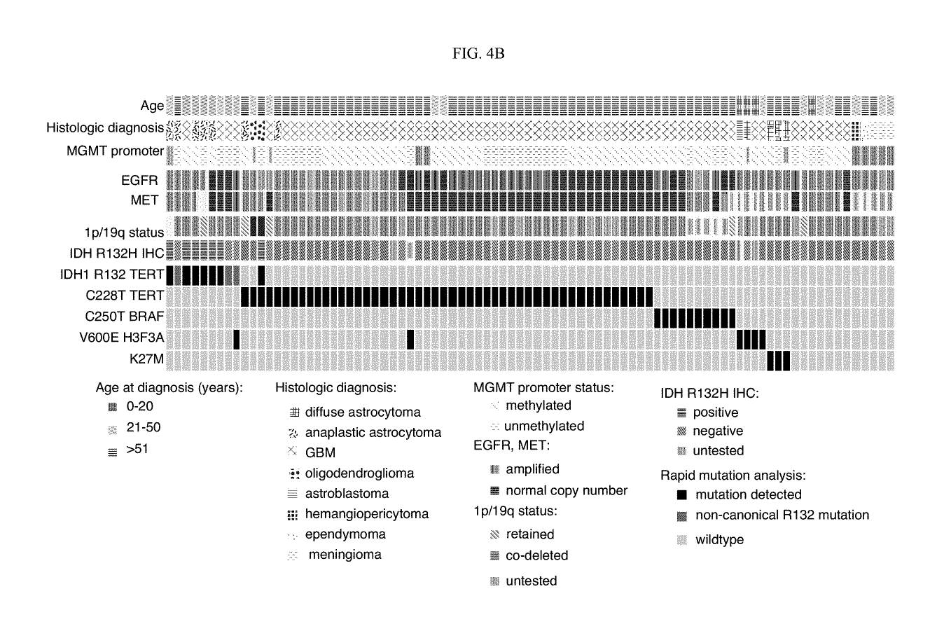

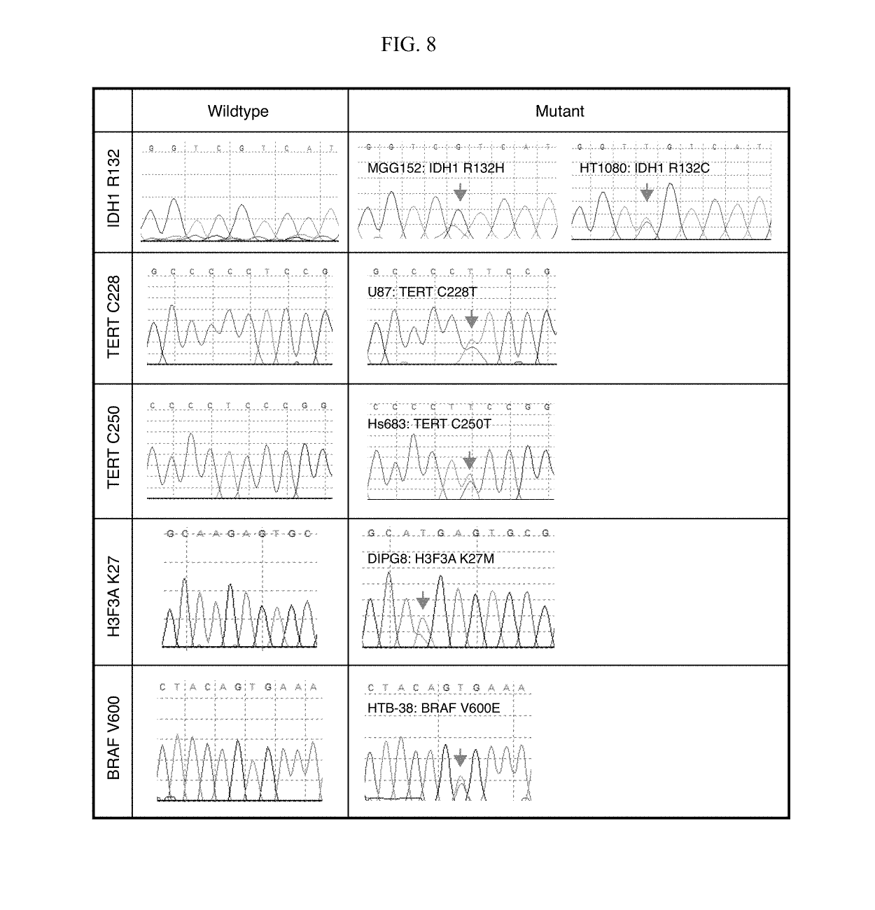

Thus, an intraoperative decision to deploy microparticles targeting NAMPT hinges on the unequivocal identification of IDH1 mutation within the tumor. To assign the molecular status of a tumor in an intraoperative timeframe, a multiplexed qPCR-based rapid genotyping assay was optimized, based upon techniques that have been previously described⁷. By suppressing exponential amplification of wild-type alleles, mutations in IDH1 could be detected at the R132 codon, the two highly-recurrent TERT promoter mutations on chromosome 5 at positions 1,295,228 and 1,295,250 (C228T and C250T, respectively), H3F3A K27M, and BRAF V600E at an allelic fraction of 1% within 27 minutes (FIG. 4A, FIGS. 8-11). The sensitivity of this method surpasses the mutant allele fraction observed in infiltrative brain tumor specimens analyzed by next generation sequencing [IDH1 R132 mutations median 32% (5-52%), TERT promoter mutation 23% (12-61%), BRAF V600E 290% (6-39%), Table 1]. Together with a rapid extraction protocol of specimen of 5 minutes (Methods), the tissue-to-genotype time is under 35 minutes.

This assay was then validated on 87 clinically annotated tumor specimens (FIG. 4B). Representing a distillation of large-scale genomic surveys of gliomas over the last decade, the clonal presence and restricted distribution of the mutations in this panel in gliomas allows for unequivocal subclass assignment for most tumors. In the cohort, 75 of 87 brain tumor samples (86%) were positively assigned by the presence of one or more mutations; when restricted to tumors with diffuse glioma histology, >90% are captured by the presence of one of these hotspot mutations. These findings strongly suggest that the combination of rapid molecular testing alongside traditional intraoperative frozen histopathology will, in the vast majority of cases, yield definitive and timely information to guide the appropriate local therapy application.

In vivo safety and efficacy of GMX-1778 sustained release microparticles.

TABLE 1

| | | Mutant allele |
|---|---|---|
| Tumor ID | Somatic mutation | fraction |
| Glioma-4762 | TERT C228T | 0.12 |
| Glioma-4639 | TERT C228T | 0.14 |
| Glioma-8661 | TERT C228T | 0.16 |
| Glioma-6880 | TERT C228T | 0.19 |
| Glioma-6273 | TERT C228T | 0.19 |
| Glioma-5634 | TERT C228T | 0.29 |
| Glioma-5646 | TERT C228T | 0.32 |
| Glioma-5963 | TERT C228T | 0.33 |
| Glioma-8115 | TERT C228T | 0.41 |
| Glioma-6162 | TERT C228T | 0.61 |
| Glioma-8267 | TERT C250T | 0.22 |
| Glioma-5491 | TERT C250T | 0.23 |
| Glioma-4920 | TERT C250T | 0.26 |
| Glioma-4476 | TERT C250T | 0.29 |
| Glioma-827 | BRAF V600E | 0.06 |
| Glioma-5000 | BRAF V600E | 0.16 |
| Glioma-1691 | BRAF V600E | 0.26 |
| Glioma-937 | BRAF V600E | 0.26 |
| Glioma-1784 | BRAF V600E | 0.29 |
| Glioma-6090 | BRAF V600E | 0.30 |
| Glioma-1972 | BRAF V600E | 0.32 |
| Glioma-5520 | BRAF V600E | 0.34 |
| Glioma-171 | BRAF V600E | 0.39 |
| Glioma-5268 | IDH1 R132S | 0.05 |
| Glioma-6380 | IDH1 R132H | 0.06 |
| Glioma-3317 | IDH1 R132H | 0.11 |
| Glioma-5083 | IDH1 R132C | 0.12 |
| Glioma-2579 | IDH1 R132G | 0.15 |
| Glioma-7815 | IDH1 R132C | 0.16 |
| Glioma-4091 | IDH1 R132C | 0.17 |
| Glioma-5939 | IDH1 R132C | 0.17 |
| Glioma-4474 | IDH1 R132C | 0.19 |
| Glioma-5318 | IDH1 R132H | 0.24 |
| Glioma-2929 | IDH1 R132L | 0.29 |
| Glioma-985 | IDH1 R132H | 0.29 |
| Glioma-6250 | IDH1 R132L | 0.29 |
| Glioma-7533 | IDH1 R132C | 0.30 |
| Glioma-4735 | IDH1 R132C | 0.30 |
| Glioma-6270 | IDH1 R132C | 0.31 |
| Glioma-7064 | IDH1 R132C | 0 33 |
| Glioma-2612 | IDH1 R132H | 0.35 |
| Glioma-6163 | IDH1 R132H | 0.36 |
| Glioma-6207 | IDH1 R132H | 0 40 |
| Glioma-3601 | IDH1 R132G | 0.40 |
| Glioma-722 | IDH1 R132H | 0.40 |
| Glioma-6376 | IDH1 R132C | 0.41 |
| Glioma-7517 | IDH1 R132H | 0.42 |
| Glioma-3986 | IDH1 R132H | 0.42 |
| Glioma-5813 | IDH1 R132C | 0.43 |
| Glioma-6000 | IDH1 R132H | 0.44 |
| Glioma-1128 | IDH1 R132C | 0.44 |
| Glioma-2010 | IDH1 R132H | 0.47 |
| Glioma-6559 | IDH1 R132C | 0.51 |
| Glioma-2669 | IDH1 R132H | 0.52 |

Table 1 shows mutant allele fraction noted in glioma specimen analyzed by clinical next generation sequencing (NGS). Retrospective analysis of allele fraction of somatic mutations observed in a cohort of glioma specimens analyzed by NGS reveals a median fraction of 32% for IDH1 R132 mutations (range 6-52%), 23% for TERT promoter mutation (12-61%), and 29% for BRAF V600E (6-39%). This established the dynamic range that is required for sensitive detection of somatic mutations in biopsy specimens of infiltrative disease.

Finally, to assess whether tumor progression can be impacted in vivo with microparticle local therapy, the sustained release formulation of GMX-1778 was tested in orthotopic glioma models. Importantly, as a control, mice receiving intracranial GMX-1778 microparticles did not display detectable local (seizures, infection) or systemic toxicities. For example, there was no detectable anemia (hemoglobin 11.6±0.4 g/dL for blank vs 11.0±1.1 g/dL for GMX-1778 microparticles). Indeed, GMX-1778 levels in brain parenchyma were below the limits of detection by LCMS at 2 or 7 days following intracranial implantation of microparticles, consistent with the sustained release of low nanomolar concentrations of compound, and subsequent clearance.

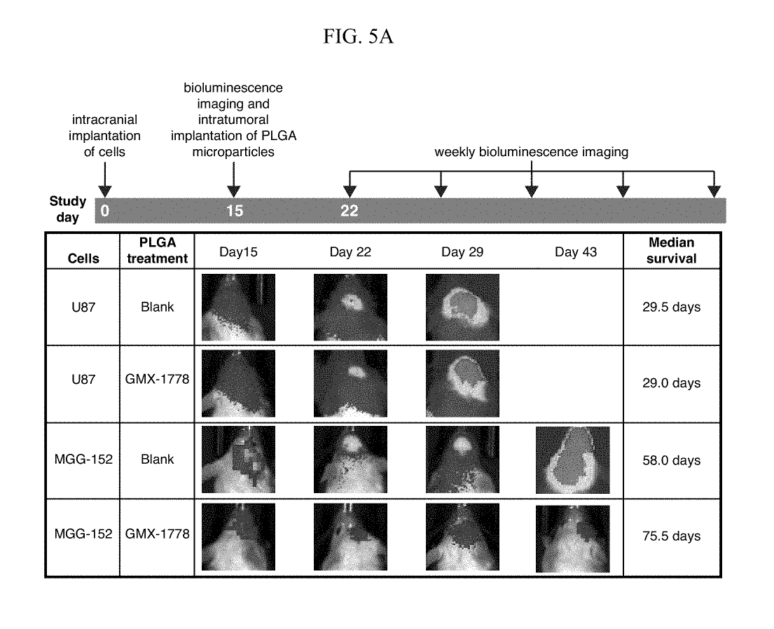

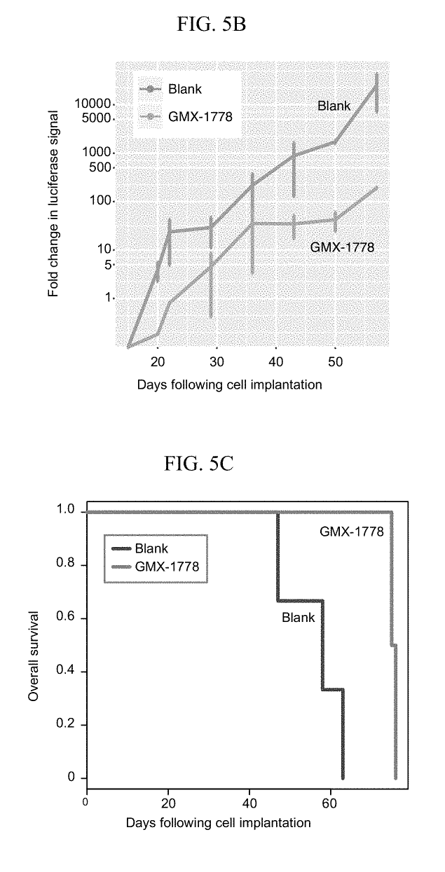

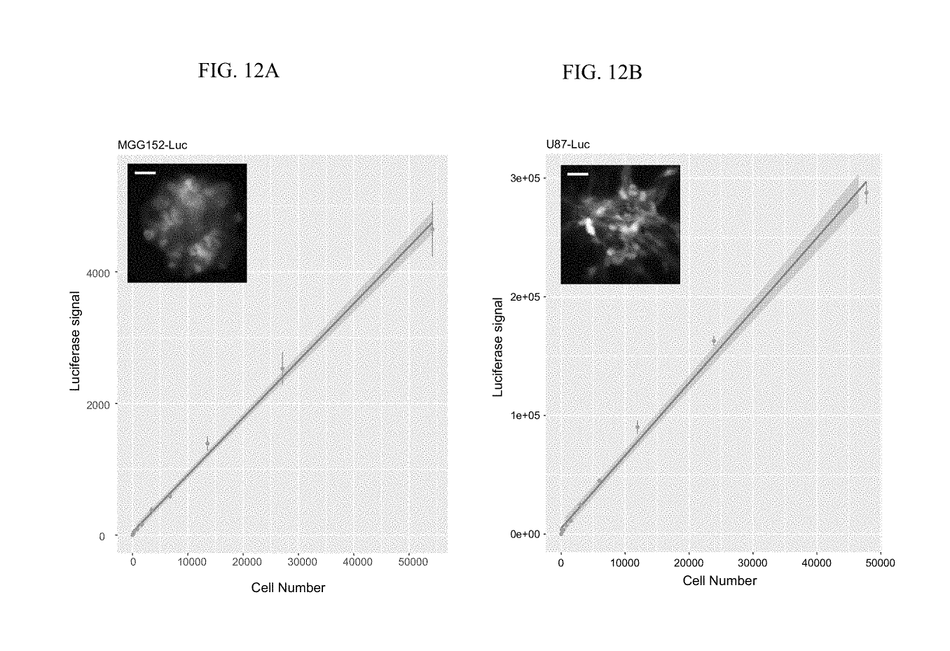

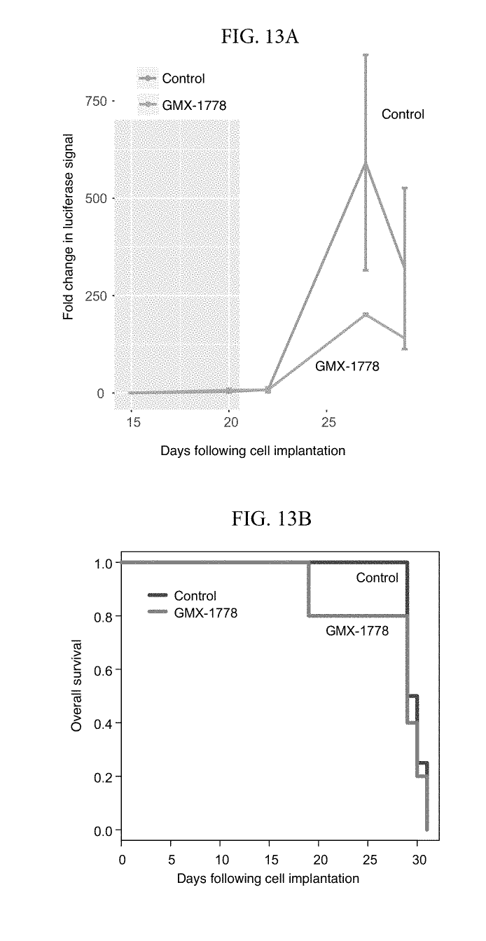

Orthotopic gliomas were then established by stereotactic intracranial implantation of MGG152 (IDH1 R132H) or U87 (IDH1 wildtype, TERT promoter mutated) glioma cells engineered to express luciferase (FIG. 12). Established tumor growth was documented by bioluminescence imaging performed 15 days following implantation, prior to stereotactic intratumoral treatment with microparticles containing GMX-1778 (FIG. 5A). Strikingly, GMX-1778 microparticles significantly decreased tumor growth in the MGG152 orthotopic model compared to blank microparticles (FIG. 5B), with a significant difference in median survival (FIG. 5C, 58±11 days vs 76±0.5 for blank, n=5, vs GMX-1778, n=6; p<0.05 Cox proportional hazard ratio). As an indicator of on-target effect, GMX-1778 microparticles did not significantly affect survival in the U87 orthotopic model (FIG. 5C, FIG. 13; 29.5 days for blank, n=4, vs 29 days for GMX-1778, n=5).

### Discussion

The rationally-directed administration of targeted chemotherapeutics to patients with tumors harboring known oncogenic alterations forms the foundation of precision oncology clinical management paradigms. With this guiding logic, systemic anti-cancer therapies targeting tumor-specific variants have had a profound impact on overall patient survival¹⁹⁻²². Many surgical oncology scenarios could benefit from improved local control with the intraoperative delivery of genotype-targeted therapies to neoplasms that have not yet progressed to regional or metastatic spread. Furthermore, systemic targeted therapies can be accompanied by unwanted systemic toxicity, such as formation of cutaneous skin cancers, hepatotoxicity, or ocular toxicity²³⁻²⁵, that can hinder their widespread use in less-advanced disease. Local administration directly at the site of disease represents an opportunity for the re-consideration of otherwise-effective anti-cancer agents that have been abandoned due to systemic toxicities.

Due to unique historical and genomic circumstances, adult diffuse gliomas exemplify the ideal oncologic lesion for the development of such genotype-based local therapy. Greater than 90% of cases are characterized by recurrent hotspot mutations in IDH1/IDH2, TERT promoter, H3F3A and BRAF, with the high frequency and mutual exclusivity of these alterations essentially serving as an internal diagnostic control, confirming the presence of tumor tissue, and positively reinforcing the histologic sub-classification²⁶. By accelerating the mutational scoring into the same timeframe alongside the intraoperative histological frozen section analysis that secures the diagnosis of glioma, the stage is then set for genotype-directed local therapy during the routine initial operative setting.

Indeed, the linchpin of the intraoperative decision to use local therapy is the timely acquisition of molecular status of the tumor. In this study, one such approach was demonstrated with the design of microparticles that secrete NAMPTi in a time-dependent manner, to exploit the metabolic vulnerability of IDH1 mutant gliomas. This designed microparticle is novel in its modulation of the metabolic microenvironment, allowing the delivery of anti-tumor therapy from the time of surgery until the initiation of subsequent adjuvant treatment. Accordingly, this approach directly addresses the natural history of this tumor, which is characterized by progression at the margin of the initial tumor despite maximal pursuit of existing standard-of-care therapies involving surgical resection, systemic chemotherapy or radiation therapy. The bio-available properties of the microparticle should nonetheless integrate seamlessly with the delivery of subsequent standard-of-care. Moreover, with recent findings of a combination metabolic effect between the oral alkylating agent temozolomide and NAMPTi in IDH1 mutant cancers²⁷, testing synergistic strategies combining local microparticle administration with oral temozolomide, or reduced systemic doses of NAMPTi, can be envisioned to further augment therapeutic benefit.

In addition to delivering NAMPTi to diffuse gliomas, this approach could be translated immediately to other tumors characterized by IDH1 mutation where local control is an important surgical goal, such as cholangiocarcinoma²⁸ or cartilaginous tumors^{29,30}. More generally, within the CNS, a precision surgical paradigm of combining molecular diagnosis with delivery of targeted therapy within the resection cavity can be facilitated for gliomas and other tumors bearing hotspot mutations captured on a diagnostic platform, such as BRAF mutant melanoma brain metastases or craniopharyngiomas. Outside the CNS, in appropriate clinical scenarios, such a pairing holds the potential to broadly extend the reach of precision therapeutics in surgical oncology.

### Methods

Retrospective Analysis of Outcomes in Patients with Diffise Astrocytoma

Case records were reviewed under approval of the Institutional Review Boards at Dana-Farber Cancer Institute, Massachusetts General Hospital and University Hospital Dresden. Patients with an IDH1/2 mutated diffuse astrocytoma diagnosed between 1988-2014 were included for analysis. Tumors with 1p/19q co-deletion were excluded. Progression was defined at the first imaging study with radiographic change. Local progression was defined as within 2 cm of the initial lesion margin and distal progression was defined as occurring farther than 2 cm.

### Pharmacokinetic Analysis of GMX-1778

GMX-1778 was dissolved in 20% captisol (CyDex) and administered by oral gavage to 6-7 week old female SCID mice at a dose of 250 mg/kg. Vehicle treated animals received similar volume of 5% dextrose prepared in 20% captisol by oral gavage. GMX-1778 treated animals were sacrificed at 30 minutes, 2 hours, 6 hours and 24 hours following treatment. Blood was collected by cardiac puncture and centrifuged at 6000 g for 5 minutes. The brain was homogenized in 3 volumes of PBS with 5% bovine serum albumin (Sigma-Aldrich). GMX-1778 was then extracted into the organic phase by a modified protocol described previously³¹. Briefly, 25 μL plasma or 100 μL brain homogenate was added to 10 μL FK-866 (250 ng/mL prepared in methanol). Then, 800 μL ethyl acetate was added and the sample was vortexed and mixed overnight. The sample was then centrifuged at 3000 rpm for 10 minutes and 600 μL supernatant was transferred to a new tube. The sample was dried overnight and reconstituted in 200 μL acetonitrile. Stock solutions of GMX-1778 and an internal standard FK-866 were prepared in acetonitrile at a concentration of 500 μg/mL. A10-point calibration curve ranging from 5 to 5000 ng/mL was prepared. Quality control samples were prepared by a similar procedure using an independent stock solution at three concentrations (25, 250, and 2500 ng/mL). For analysis, 2.5 μL sample was injected on the UPLC-ESI-MS system.

Ultra-performance LC (UPLC) separation was carried out on a Waters UPLC aligned with a Waters Xevo TQ-SMS mass spectrometer (Waters Ltd.). MassLynx 4.1 software was used for data acquisition and analysis. LC separation was performed on an Acquity UPLC High Strength Silica Trifunctional C18 (50 mm×2.1 mm; particle size, 1.8 μm) at 50° C. The mobile phase consisted of acetonitrile, 0.1% formic acid, 10 mM ammonium formate solution flowed at a rate of 0.6 mL/minute using a time and solvent gradient composition. The initial mobile phase composition (100%) was held for 1.0 minutes, and then changed to 20% over the next 0.25 minutes. Finally, over the next 0.25 minutes the composition was brought to 0% and held for 0.5 minutes, and finally shifted to the initial composition of 100% over the next 0.25 minutes and held constant until the end of the run for column equilibration. The total run time was 4.0 minutes, and sample injection volume was 2.5 μL. The mass spectrometer was operated in the multiple reaction monitoring mode. Sample introduction and ionization was carried out by electron spray ionization (ESI) in the positive-ion mode.

### Systemic Toxicity Analysis of Oral GMX-1778

Adult SCID female mice of 6-7 weeks were administered GMX-1778 daily for 5 days by oral gavage (250 mg/kg). Body weights were recorded prior to administration and at the end of the study. Blood samples were analyzed for complete blood count by HemaTrue (Heska Lab Systems) and chemistry panel by DriChem (Heska Lab Systems). Liver and heart were fixed in formalin and analyzed for histologic toxicity by hematoxylin and eosin (H&E) staining. Eyes were removed by enucleation and placed in Davidson's fixative for 24 hours prior to formalin and subsequently analyzed for toxicity on H&E slides.

### Synthesis of GMX-1778 Microparticles

PLGA microparticles loaded with GMX-1778 were prepared by a single emulsion technique. The polymer (80 mg) was placed in 2 mL of chloroform and allowed to dissolve for 1-2 h. GMX-1778 (20 mg or 8 mg) was dissolved in 2 mL of a mixture of chloroform and methanol (4:1 v/v). The polymer and drug solutions were then mixed and added to an aqueous poly(vinyl alcohol) solution (15 mL, 2.5% w/w). The mixture was then either vortexed (for Formulations A-D) or homogenized (for Formulations E-I) for five minutes. The resultant emulsion was further diluted in 25 mL 2.5% w/w aqueous poly(vinyl alcohol) solution and the organic solvents were removed either by using a rotavap under high vacuum for 2 hours or by stirring overnight under ambient conditions. Following solvent evaporation, the microparticle dispersion was filtered through a 70 μm filter, and the filtrate was washed four times by centrifugation (4000 RPM, 10 minutes, 4° C.). For Formulations A-F, all four washes were performed with 50 mL deionized water. For Formulation G, H and I, the particles were first washed twice with 50 mL of citrate buffer, 10% sodium dodecyl sulfate or 10% Tween 20 respectively, followed by two washes with deionized water. After the final wash, the microparticles were dispersed in 1 mL deionized water, frozen to −80° C., and lyophilized for 48 h (Labconco, USA).

### Physicochemical Characterization of Microparticles

The size and morphology of microparticles were analyzed using a JEOL 5600LV scanning electron microscope (JEOL USA, Inc., USA). Dried microparticles were first spread over a carbon conductive tape attached to an aluminum stub. The particles were then sputter coated with Hummer sputter coating system (Anatech, USA, USA) using a gold/palladium source. Size of the microparticles was assessed using ImageJ 1.51K software.

To determine drug loading, drug was extracted overnight by dispersing the microparticles in methanol. On the next day, particles were separated by centrifugation (4000 RPM, 5 minutes) and drug concentration in the supernatant was measured using high performance liquid chromatography (HPLC). HPLC was performed on an Agilent 1260 Infinity HPLC system (Agilent Technologies Inc., USA) equipped with Model 1260 quaternary pump, Model 1260 Hip ALS autosampler, Model 1290 thermostat, and Model 1260 TCC control module. Five microliters of the sample were injected onto a Poroshell 120 EC-C18 column (4.6×50 mm, 2.7 μm). The mobile phase consisted of a mixture of acetonitrile and 0.1% formic acid flown at 1.2 mL/minute. The mobile phase composition changed from 100% acetonitrile at time 0 to 100% aqueous phase over 8 minutes. Drug was detected using a Model 1260 diode array detector at λₘₐₓ=280 nm.

The release of GMX-1778 from microparticles was measured in artificial cerebrospinal fluid. Microparticles were dispersed in 1 mL release media, and placed in an incubator shaker at 37° C. and 100 RPM. At various times, particles were separated using centrifugation and the release media was collected for further processing. The particle pellet was suspended in fresh media. The release media was extracted with ethyl acetate for 24 hours. After 24 hours, the ethyl acetate layer was carefully transferred to a new vial and evaporated under reduced pressure. The residue was dissolved in 0.1 mL methanol and analyzed by HPLC as described above. Standards were prepared using an identical procedure after dissolving known amounts of the drug in the release media.

### In Vitro Analysis of NAMPT Inhibitor Toxicity

Previously described cell lines were cultured in 96-well plates: MGG152 (human glioma, IDH1 R132H, 1×10⁴ cells), MGG119 (human glioma, IDH1 R132H, 1×10⁴ cells), HT1080 (human chondrosarcoma, IDH R132C, 1.5×10³ cells), 30T (human melanoma, IDH1 R132C, 1×10⁴ cells), U87 (human glioblastoma, TERT C228T, 1×10⁴ cells), Hs683 (human glioblastoma, TERT C250T, 1×10⁴ cells), T98G (human glioblastoma, TERT C250T, 1×10⁴ cells), DIPG7 (human diffuse pontine glioma, H3F3A K27M, 1×10⁴ cells), and Saga27 (human diffuse pontine glioma, H3F3A K27M, 1×10⁴ cells). The following day after cells were plated, microparticles were added to the media. Microparticles were reconstituted in DMEM and briefly sonicated in a water bath. Given the noted drug release kinetics from the microparticles, a total of 1 μM drug loaded in microparticles was added to the well to obtain sustained ˜100 nM drug concentration over the first 24-72 hours. A similar amount of drug-free microparticles was added to separate wells used for normalization. Cell viability was assessed using CellTiter-Glo (Promega). NAD+ and NADH levels were quantified by NAD/NADH-Glo Assay (Promega) according to manufacturer's recommendations.

Preparation of Genomic Extracts from Tumor Specimens

DNA was extracted from formalin-fixed, paraffin-embedded (FFPE) specimens using the QiaAMP kit according to the manufacturer's recommendations (Qiagen). Fresh specimens were extracted by a modified ChargeSwitch protocol (Invitrogen). Briefly, tissue was snap frozen, thawed and pulverized by a sterile pestle before the addition of 1 mL of warmed lysis buffer containing 100 units of Proteinase K. The tissue was incubated at 55° C. for 3 minutes with intermittent mixing prior to addition of 0.2 mL of purification buffer and 0.04 mL of magnetic ChargeSwitch beads. The beads were incubated with lysed tissue for 1 minute, washed two times in 1 mL of wash buffer and eluted in 50 μL of elution buffer. Concentration of genomic extract was quantified by PicoGreen dsDNA assay (Life Technologies).

### Quantitative PCR Assay

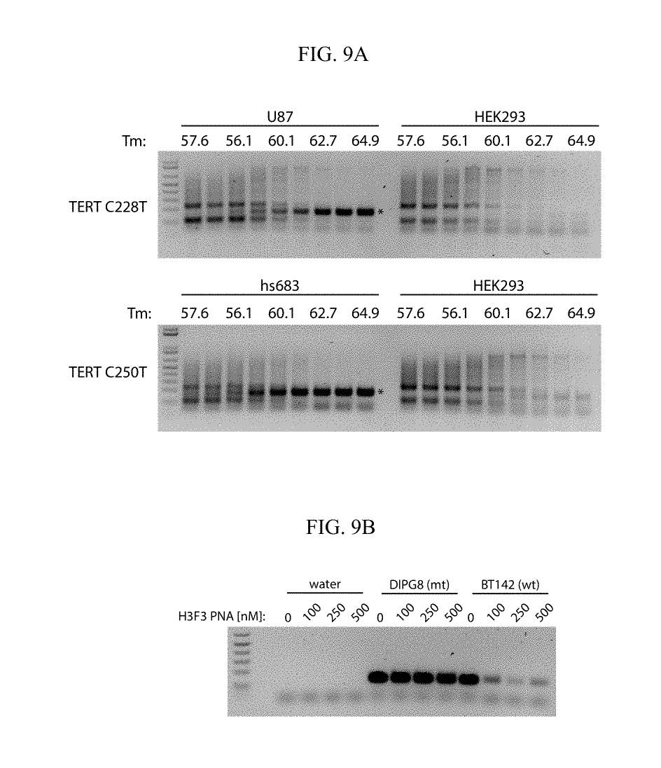

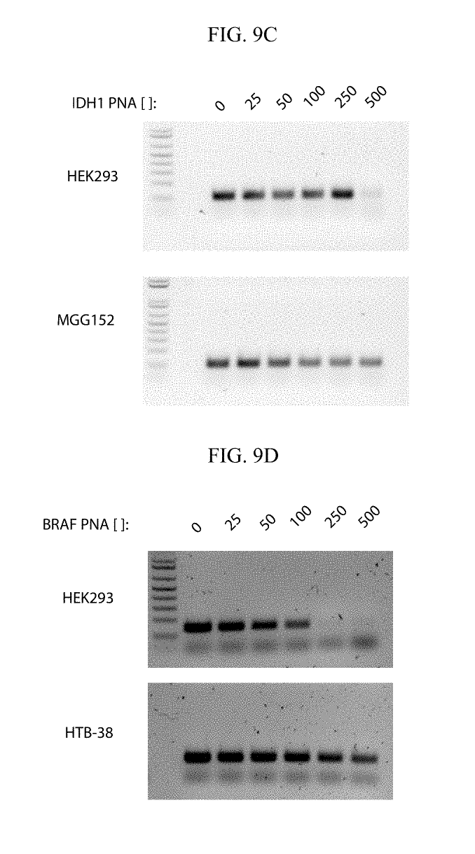

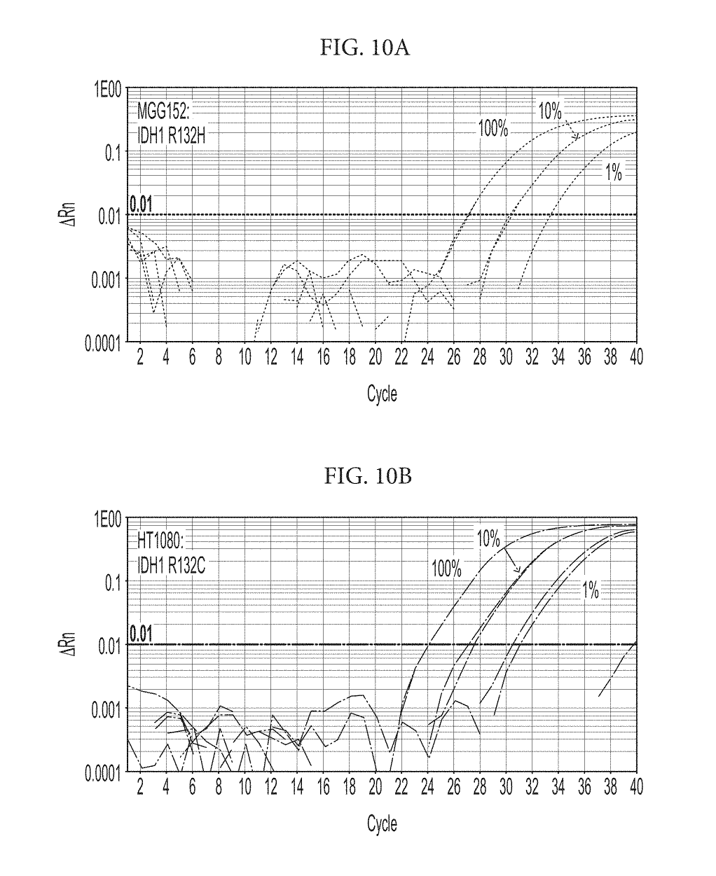

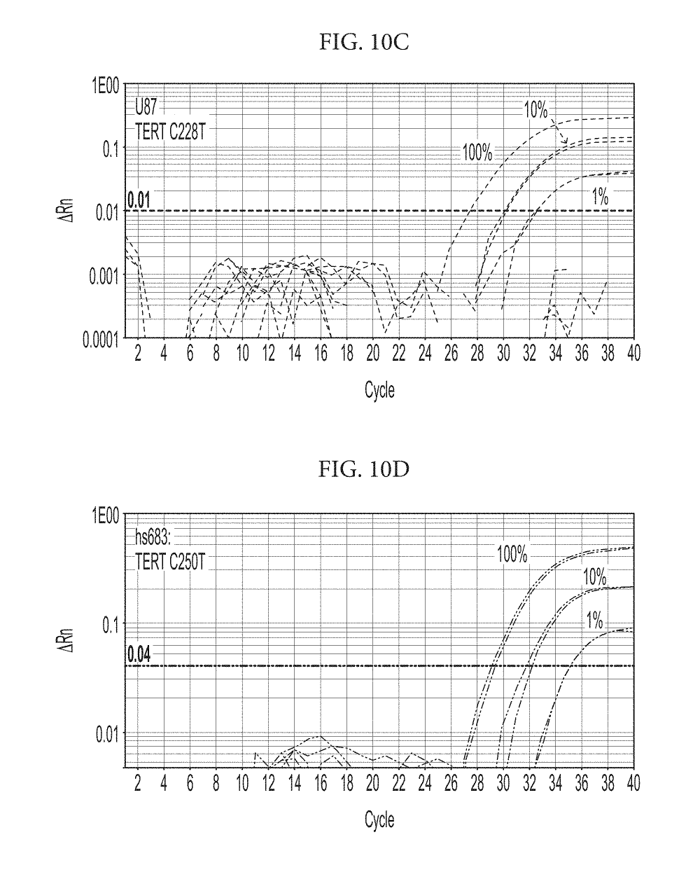

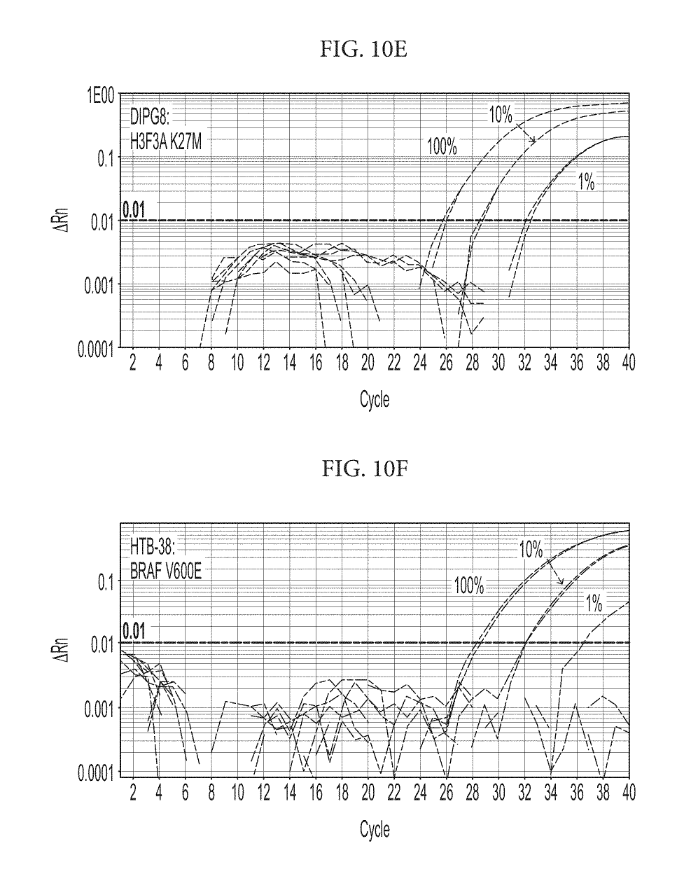

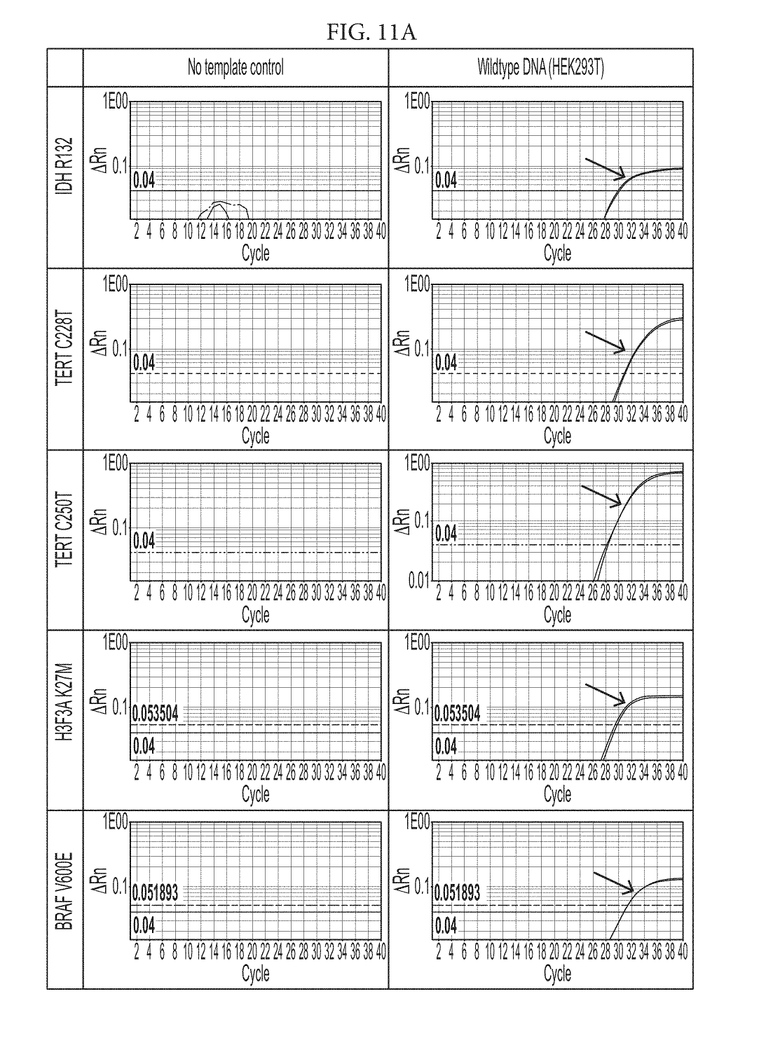

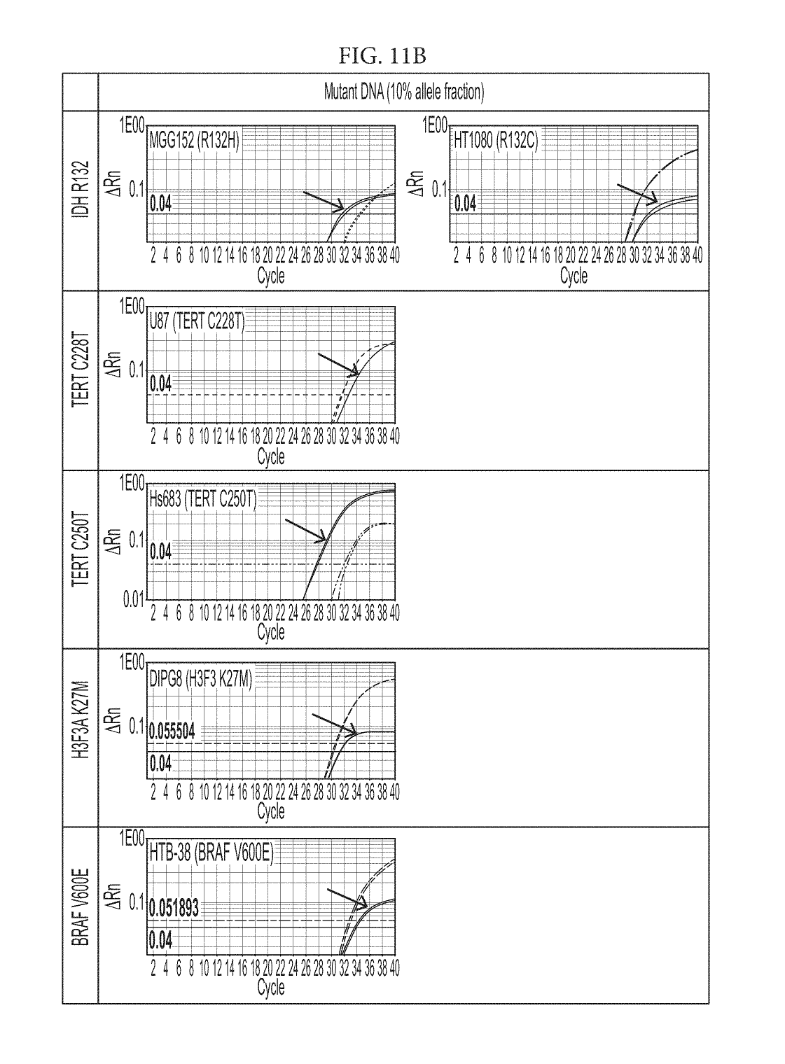

5′ Nuclease probes against mutant alleles and corresponding PNA oligonucleotide blockers against wild-type alleles were designed to detect IDH1 R132H, R132C, R132G, R132S and R132L, TERT promoter mutations on chromosome 5 at positions 1,295,228 and 1,295,250 based on human genome reference version 19 (referred to hereafter as TERT C228T or TERT C250T), H3F3A K27M and BRAF V600E (Table 2). TERT assays were performed using Kapa Biosystems 2G Robust PCR polymerase in buffer A with enhancer, H3F3A assay was performed using the Kapa 2G Robust kit with buffer A without enhancer, and the IDH1 and BRAF assays were run in the ABI Taqman Gene Expression master mix with 1U platinum Taq polymerase (Invitrogen) containing oligonucleotide concentrations as outlined in Table 2. To ensure the integrity of the assay, every qPCR reaction was run in duplicate and included positive controls from genomic extracts of MGG152 (IDH1 R132H), HT1080 (IDH1 R132C), U87 (TERT C228T), Hs683 (TERT C250T), DIPG8 (H3F3A K27M), and HTB-38 (BRAF V600E) (FIG. 8). Genomic extract from HEK293T cells served as wild-type control. Optimal sensitivity was achieved through the use of distinct peptide nucleic acid blocking oligonucleotides directed to the wild-type alleles (FIG. 9). The qPCR reactions were run on a QuantStudio7 instrument on a fast heat block (Applied Biosystems). The lid was preheated and PCR cycling times were 99° C. for 30 seconds followed by 40 cycles at 95° for 5 seconds and 63.5° C. for 10 seconds. Ramp rate was set to 3.4° C./second. Reactions were performed with at least 10 ng of whole genomic extract in 10 μL reaction volume. In this optimized assay, serial dilutions representing 1%-100% of positive control genomic extracts diluted in negative control extracts could be detected (FIG. 10). Internal control for the assay was developed for detection of GAPDH to ensure assay integrity (FIGS. 11A-11B).

TABLE 2

| | | Forward Primer | | Reverse Primer | |
|---|---|---|---|---|---|
| | | | Concentration | | Concentration |
| Gene | sSNV | Sequence | (nM) | Sequence | (nM) |
| TERT | C228T | 5′- | 500 | 5′- | 500 |
| | | CACGTGCGCAGCA | | CTTCACCTTCCAGCTCC | |
| | | GGACGCAG3′ (SEQ | | GCCTC-3′ (SEQ ID NO: | |
| | | ID NO: 1) | | 17) | |
| TERT | C250T | 5′- | 500 | 5′- | 500 |
| | | CACGTGCGCAGCA | | CTTCACCTTCCAGCTCC | |
| | | GGACGCAG3′ (SEQ | | GCCTC-3′ (SEQ ID NO: | |
| | | ID NO: 1) | | 17) | |
| IDH1 | R132H | 5′- | 500 | 5′- | 500 |
| | | GCCAACATGACTT | | GCACGGTCTTCAGAGAA | |
| | | ACTTGATCCCCA3′ | | GCCA3′ | |
| | | (SEQ ID NO: 2) | | (SEQ ID NO: 18) | |
| IDH1 | R132C | 5′- | 500 | 5′- | 500 |
| | | GCCAACATGACTT | | GCACGGTCTTCAGAGAA | |
| | | ACTTGATCCCCA3′ | | GCCA3′ | |
| | | (SEQ ID NO: 3) | | (SEQ ID NO: 18) | |
| IDH1 | R132G | 5′- | 500 | 5′- | 500 |
| | | GCCAACATGACTT | | GCACGGTCTTCAGAGAA | |
| | | ACTTGATCCCCA3′ | | GCCA3′ | |
| | | (SEQ ID NO: 3) | | (SEQ ID NO: 18) | |
| IDH1 | R132S | 5′- | 500 | 5′- | 500 |
| | | GCCAACATGACTT | | GCACGGTCTTCAGAGAA | |
| | | ACTTGATCCCCA3′ | | GCCA3′ | |
| | | (SEQ ID NO: 3) | | (SEQ ID NO: 18) | |
| IDH1 | R132L | 5′- | 500 | 5′- | 500 |
| | | GCCAACATGACTT | | GCACGGTCTTCAGAGAA | |
| | | ACTTGATCCCCA3′ | | GCCA3′ | |
| | | (SEQ ID NO: 3) | | (SEQ ID NO: 18) | |
| H3F3A | K27M | 5′- | 500 | 5′- | 500 |
| | | AATCGACCGGTGG | | ACATACAAGAGAGACTT | |
| | | TAAAGCA-3′ | | TGTCCCA-3′ | |
| | | (SEQ ID NO: 4) | | (SEQ ID NO: 19) | |
| BRAF | V600E | 5′- | 500 | 5′-TGC TTG | 500 |
| | | CAGACAACTGTTC | | C*TC*TGA*TAG GAA | |
| | | AAACTGATGGGAC | | AA*TGA-3′ (SEQ ID | |
| | | CCAC-3′ | | NO: 20) | |
| | | (SEQ ID NO: 5) | | | |
| GAPDH | | 5′- | varies | 5′- | varies |
| | | TGGGGGTTCTGGG | | ATGATGTTCTGGAGAGC | |
| | | GACTGGCT-3′ (SEQ | | CCCGC-3′ | |
| | | ID NO: 6) | | (SEQ ID NO: 21) | |
| | | 5′ Nuclease Probes | | PNA blocker | |
| | | | Concentration | | Concentration |
| Gene | sSNV | Sequence | (nM) | Sequence | (nM) |
| TERT | C228T | 5′-FAM- | 250 | 5′- | 150 |
| | | CCAGCCCC+T+TCC | | CCCAGCCCCCTCCGGGC | |
| | | GGGCCC-3BHQ-3′ | | CC-3′ (SEQ ID | |
| | | (SEQ ID NO: 7) | | NO: 22) | |
| TERT | C250T | 5′-FAM- | 250 | 5′- | 250 |
| | | CGACCCC+T+T+CC | | CCCGACCCCTCCCGGGT | |
| | | GGGTCCC-3BHQ-3′ | | CCCC-3′ | |
| | | (SEQ ID NO: 8) | | (SEQ ID NO: 23) | |
| IDH1 | R132H | 5′-FAM- | 250 | 5′- | 500 |
| | | AG+G+T+C+A+T+C | | AGGTCGTCATGCTTATG | |
| | | AT+G+C+TTA-Dab-3′ | | G-3′ (SEQ ID NO: 24) | |
| | | (SEQ ID NO: 9) | | | |
| IDH1 | R132C | 5′-MAXN- | 250 | 5′- | 500 |
| | | AG+G+T+T+G+T+C+ | | AGGTCGTCATGCTTATG | |
| | | AT+G+CTTA-Dab-3′ | | G-3′ (SEQ ID NO: 24) | |
| | | (SEQ ID NO: 10) | | | |
| IDH1 | R132G | 5′-MAXN- | 250 | 5′- | 500 |
| | | AGGT+G+G+T+CAT+ | | AGGTCGTCATGCTTATG | |
| | | GC+T+TA-Dab-3′ | | G-3′ (SEQ ID NO: 24) | |
| | | (SEQ ID NO: 11) | | | |
| IDH1 | R132S | 5′-MAXN- | 250 | 5′- | 500 |
| | | AG+GT+A+G+T+CA+ | | AGGTCGTCATGCTTATG | |
| | | T+G+CT+TA-Dab-3′ | | G-3′ (SEQ ID NO: 24) | |
| | | (SEQ ID NO: 12) | | | |
| IDH1 | R132L | 5′-MAXN- | 250 | 5′- | 500 |
| | | AG+G+T+C+T+T+CA | | AGGTCGTCATGCTTATG | |
| | | T+G+C+TTA-Dab-3′ | | G-3′ (SEQ ID NO: 24) | |
| | | (SEQ ID NO: 13) | | | |
| H3F3A | K27M | 5′-FAM-CT+C+GC+A+ | 250 | 5′-GCTCGCAAGAGTGCG- | 250 |
| | | TG+AG+TGC-Dab- | | 3′ (SEQ ID NO: 25) | |
| | | 3′ (SEQ ID NO: 14) | | | |
| BRAF | V600E | 5′-FAM- | 250 | 5′- | 250 |
| | | CTA+G+CTA+CA+G+ | | CTAGCTACAGTGAAATC | |
| | | A+GAAAT+CTCG- | | TCG-3′ | |
| | | Dab-3′ | | (SEQ ID NO: 26) | |
| | | (SEQ ID NO: 15) | | | |
| GAPDH | | 5′-Cy5- | varies | | |
| | | CACA+GTC+CAT+G | | | |
| | | C+CATCA+CT+GC- | | | |
| | | IowaBlack-3′ (SEQ ID | | | |
| | | NO: 16) | | | |

Table 2 shows a list of primers, detection probes and modified oligonucleotide blockers. Modified locked nucleic acids are indicated by a preceding “+”. Modified thymine bases with 5-hydroxybutynyl-2′-deoxyuridine to stabilize oligonucleotide hybridization are indicated by a preceding “*”.

### Expression of Luciferase in Human Glioma Cell Lines

Lentivirus for luciferase expression was generated by transfecting HEK293T cells with pCMV-VSV-G, pCMV-dR8.2 and plasmid with luciferase and mCherry constructs using Fugene (Promega). Media was changed at 24 hours following transfection and then conditioned media was collected after an additional 24 hours. MGG152 and U87 cells were infected with this conditioned media at a 1:1 dilution in plain medium. Media was changed at 6 hours and subsequently passaged. Cells expressing the luciferase construct were selected by FACS based on mCherry expression. Luciferase activity was measured by Luciferase Assay reagent (Promega) in serial dilutions to 5.4×10⁴ cells.

### In Vivo Activity of Sustained Release Microparticle NAMPT Inhibitor Formulation

Adult 6-7 week old female SCID mice underwent stereotactic implantation of 2×10⁵ MGG152-Luciferase or 2×10⁵ U87-Luciferase in the right striatum. Bioluminescence imaging was performed at 15 days following cell injection. Briefly, mice were anesthesized with isoflurane and injected with 4.5 mg d-luciferin. Animals were then imaged by Spectral Ami HTX (Spectral Instruments Imaging) with luminescence exposure times of 1 second and 0.5 seconds at 5 minutes intervals until peak photon/second intensity within a box drawn around the mouse cranium was reached. Mice with detectable intracranial luciferase expression at this time point were randomized to be treated with intratumoral stereotactic implantation of microparticles with GMX-1778 or similarly prepared drug-free microparticles. The final microparticle drug concentration was based on an estimated tumor size of 3 mm diameter, corresponding to a volume of ˜14 μL with a total drug concentration of 5 μM contained in the injected microparticles. The microparticles were reconstituted so that this concentration could be administered in a 2 μL volume. Bioluminescence imaging was performed on a weekly basis with intensity measured as photon intensity collected from the entire cranium.

### REFERENCES

1. Louis, D. N. et al. The 2016 World Health Organization Classification of Tumors of the Central Nervous System: a summary. Acta Neuropathol. (Berl.) 131, 803-820 (2016).
2. Yan, H. et al. IDH1 and IDH2 mutations in gliomas. N. Engl. J Med. 360, 765-773 (2009).
3. Cancer Genome Atlas Research Network et al. Comprehensive, Integrative Genomic Analysis of Diffuse Lower-Grade Gliomas. N. Engl. J. Med. 372, 2481-2498 (2015).
4. Schwartzentruber, J. et al. Driver mutations in histone H3.3 and chromatin remodelling genes in paediatric glioblastoma. Nature 482, 226-231 (2012).
5. Dahiya, S. et al. BRAF-V600E mutation in pediatric and adult glioblastoma. Neuro-Oncol. 16, 318-319 (2014).
6. Santagata, S. et al. Intraoperative mass spectrometry mapping of an onco-metabolite to guide brain tumor surgery. Proc. Natl. Acad. Sci. U.S.A 111, 11121-11126 (2014).
7. Shankar, G. M. et al. Rapid Intraoperative Molecular Characterization of Glioma. JAMA Oncol. (2015). doi:10.1001/jamaoncol. 2015.0917
8. Beiko, J. et al. IDH1 mutant malignant astrocytomas are more amenable to surgical resection and have a survival benefit associated with maximal surgical resection. Neuro-Oncol. 16, 81-91 (2014).
9. Kawaguchi, T. et al. Impact of gross total resection in patients with WHO grade III glioma harboring the IDH 1/2 mutation without the 1p/19q co-deletion. J. Neurooncol. 129, 505-514 (2016).
10. Cairncross, J. G. et al. Benefit from procarbazine, lomustine, and vincristine in oligodendroglial tumors is associated with mutation of IDH. J. Clin. Oncol. Off. J. Am. Soc. Clin. Oncol. 32, 783-790 (2014).
11. Buckner, J. C. et al. Radiation plus Procarbazine, CCNU, and Vincristine in Low-Grade Glioma. N. Engl. J. Med. 374, 1344-1355 (2016).
12. Baumert, B. G. et al. Temozolomide chemotherapy versus radiotherapy in high-risk low-grade glioma (EORTC 22033-26033): a randomised, open-label, phase 3 intergroup study. Lancet Oncol. 17, 1521-1532 (2016).
13. Seltzer, M. J. et al. Inhibition of glutaminase preferentially slows growth of glioma cells with mutant IDH1. Cancer Res. 70, 8981-8987 (2010).
14. Rohle, D. et al. An inhibitor of mutant IDH1 delays growth and promotes differentiation of glioma cells. Science 340, 626-630 (2013).
15. Grassian. A. R. et al. IDH1 mutations alter citric acid cycle metabolism and increase dependence on oxidative mitochondrial metabolism. Cancer Res. 74, 3317-3331 (2014).
16. Saha, S. K. et al. Isocitrate Dehydrogenase Mutations Confer Dasatinib Hypersensitivity and SRC Dependence in Intrahepatic Cholangiocarcinoma. Cancer Discov. 6, 727-739 (2016).
17. Tateishi, K. et al. Extreme Vulnerability of IDH1 Mutant Cancers to NAD+ Depletion. Cancer Cell 28, 773-784 (2015).
18. Sampath, D., Zabka, T. S., Misner, D. L., O'Brien, T. & Dragovich, P. S. Inhibition of nicotinamide phosphoribosyltransferase (NAMPT) as a therapeutic strategy in cancer. Pharmacol. Ther. 151, 16-31 (2015).
19. Lynch, T. J. et al. Activating mutations in the epidermal growth factor receptor underlying responsiveness of non-small-cell lung cancer to gefitinib. N. Engl. J. Med. 350, 2129-2139(2004).
20. Motzer, R. J. et al. Sunitinib versus interferon alfa in metastatic renal-cell carcinoma. N. Engl. J. Med. 356, 115-124 (2007).
21. Hyman, D. M. et al. Vemurafenib in Multiple Nonmelanoma Cancers with BRAF V600 Mutations. N. Engl. J. Med. 373, 726-736 (2015).
22. Long, G. V. et al. Overall Survival and Durable Responses in Patients With BRAF V600-Mutant Metastatic Melanoma Receiving Dabrafenib Combined With Trametinib. J. Clin. Oncol. Off. J. Am. Soc. Clin. Oncol. 34, 871-878 (2016).
23. Rinehart, J. et al. Multicenter phase II study of the oral MEK inhibitor, CI-1040, in patients with advanced non-small-cell lung, breast, colon, and pancreatic cancer. J. Clin. Oncol. Off. J. Am. Soc. Clin. Oncol. 22, 4456-4462 (2004).
24. Wassmann, B. et al. Alternating versus concurrent schedules of imatinib and chemotherapy as front-line therapy for Philadelphia-positive acute lymphoblastic leukemia (Ph+ ALL). Blood 108, 1469-1477 (2006).
25. Flaherty, K. T. et al. Combined BRAF and MEK inhibition in melanoma with BRAF V600 mutations. N. Engl. J. Med. 367, 1694-1703 (2012).
26. Abudumijiti, A. et al. Adult IDH1 Wild-type Lower-grade Gliomas Should Be Further Stratified. Neuro-Oncol. (2017). doi:10.1093/neuonc/nox078
27. Tateishi, K. et al. The Alkylating Chemotherapeutic Temozolomide Induces Metabolic Stress in IDH1-Mutant Cancers and Potentiates NAD(+) Depletion-Mediated Cytotoxicity. Cancer Res. 77, 4102-4115 (2017).
28. Farshidfar, F. et al. Integrative Genomic Analysis of Cholangiocarcinoma Identifies Distinct IDH-Mutant Molecular Profiles. Cell Rep. 19, 2878-2880 (2017).
29. Amary, M. F. et al. Oilier disease and Maffucci syndrome are caused by somatic mosaic mutations of IDH1 and IDH2. Nat. Genet. 43, 1262-1265 (2011).
30. Pansuriya, T. C. et al. Somatic mosaic IDH1 and IDH2 mutations are associated with enchondroma and spindle cell hemangioma in Ollier disease and Maffucci syndrome. Nat. Genet. 43, 1256-1261 (2011).
31. Wang, T., Oberoi, R. K. & Elmquist, W. F. Determination of cediranib in mouse plasma and brain tissue using high-performance liquid chromatography-mass spectrometry. J. Chromatogr. B Analyt. Technol. Biomed. Life. Sci. 879, 3812-3817 (2011).

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A pharmaceutical composition comprising
a population of particles coupled to a nicotinamide adenine dinucleotide (NAD) biosynthesis inhibitor.

2.-6. (canceled)

7. The pharmaceutical composition of claim 1, wherein the NAD biosynthesis inhibitor is released from the population of particles with a sustained release profile.

8. (canceled)

9. The pharmaceutical composition of claim 1, wherein the population of particles comprises a population of microparticles or a population of nanoparticles.

10.-12. (canceled)

13. The pharmaceutical composition of claim 1, wherein the NAD biosynthesis inhibitor is a nicotinamide phosphoribosyltransferase (NAMPT) inhibitor.

14. (canceled)

15. (canceled)

16. A method for local administration of a metabolic inhibitor, the method comprising
locally administering a therapeutically effective amount of the pharmaceutical composition of claim 1 to a subject in need thereof.

17. (canceled)

18. The method of claim 16, wherein the metabolic inhibitor is a NAD biosynthesis inhibitor.

19. The method of claim 16, wherein the subject has or is suspected of having a cancer.

20. The method of claim 19, wherein the cancer is characterized by the presence of one or more mutations in a nucleotide sequence of an isocitrate dehydrogenase 1 gene (IDH1), a histone H3.3 gene (H3F3A), and/or a B-Raf gene (BRAF).

21. The method of claim 20, wherein the one or more mutations in the nucleotide sequence of IDH1 encode an IDH1 protein variant, wherein the IDH1 protein variant is IDH1 R132H, IDH1 R132C, IDH1 R132G, IDH1 R132S, or IDH1 R132L;
the one or more mutations in the nucleotide sequence of H3F3A encodes a H3F3A protein variant, wherein the H3F3A protein variant is H3F3A K27M; and/or
the one or more mutations in the nucleotide sequence of BRAF encodes a B-Raf variant, wherein the B-Raf variant is B-Raf V600E.

22.-25. (canceled)

26. The method of claim 19, wherein the cancer is a glioma.

27. The method of claim 26, wherein the glioma is selected from the group consisting of high grade glioma, diffuse astrocytoma, oligodendroglioma, oligoastrocytoma, secondary glioblastoma, primary glioblastoma, and diffuse intrinsic pontine glioma.

28. The method of claim 16, wherein
(i) the administering is by intracerebral implantation in the subject;
(ii) the subject is undergoing a surgical resectioning procedure;
(iii) the pharmaceutical composition is implanted at the tumor margin; and/or
(iv) the method further comprises administering to the subject a therapeutically effective amount of one or more additional cancer therapeutics.

29.-33. (canceled)

34. A diagnostic method, the method comprising
obtaining a sample from a subject; and
detecting one or more mutations in a target nucleic acid in the sample;
wherein the detecting is performed by
isolating a nucleic acid comprising the target nucleic acid from the sample, and
analyzing the nucleic acid for the presence of one or more allele-specific mutations; and
wherein the detecting is performed in an intraoperative timeframe.

35. The method of claim 34, wherein the intraoperative timeframe is less than 1 hour, less than 35 minutes, or less than 30 minutes.

36.-48. (canceled)

49. The method of claim 34, wherein the analyzing comprises performing a polymerase chain reaction to detect or amplify the one or more mutation in the target nucleic acid.

50. The method of claim 49, wherein the polymerase chain reaction comprises
(a) denaturing the nucleic acid isolated from the sample;
(b) annealing
(i) a forward primer comprising a nucleotide sequence that hybridizes to a first region on a sense strand of the target nucleic acid,
(ii) a reverse primer comprising a nucleotide sequence that hybridizes to a second region on an antisense strand of the target nucleic acid,
(iii) a probe comprising a nucleotide sequence that is complementary to a mutant allele sequence of the target nucleic acid and located within the region amplified by the forward primer and the reverse primer, and
(iv) a peptide nucleic acid (PNA) blocker that hybridizes to a wild-type allele of the target nucleic acid, the PNA blocker comprising a nucleotide sequence that blocks amplification of the wild-type allele, and does not block amplification of the mutant allele, wherein the PNA blocker hybridizes to a region located within the region amplified by the forward and reverse primer;
(c) amplifying a DNA amplicon comprising the mutant allele in the first target nucleic acid;
(d) detecting the mutant allele in the target nucleic acid; and
(e) quantifying the amount of the mutant allele in the target nucleic acid in the sample.

51.-53. (canceled)

54. The method of claim 34, wherein (i) the target nucleic acid comprises a portion of the nucleotide sequence of an isocitrate dehydrogenase 1 (IDH1) gene;
(ii) the target nucleic acid comprises a portion of the nucleotide sequence of a telomerase reverse transcriptase (TERT) gene or the TERT promoter;
(iii) the target nucleic acid comprises a portion of the nucleic acid sequence of a nucleotide sequence of a histone H3.3 gene (H3F3A); and/or
(iv) the target nucleic acid comprises a portion of the nucleic acid sequence of a nucleotide sequence of a B-Raf gene (BRAF).

55. (canceled)

56. The method of claim 54, wherein (i) the nucleotide sequence of the forward primer is provided by SEQ ID NO: 2 or 3;
(ii) the nucleotide sequence of the reverse primer is provided by SEQ ID NO: 18;
(iii) the nucleotide sequence of the probe is provided by any one or more of SEQ ID NOs: 9-13; and/or
(iv) the nucleotide sequence of the PNA blocker is provided by SEQ ID NO: 24.

57.-61. (canceled)

62. The method of claim 54, wherein (i) the nucleotide sequence of the forward primer is provided SEQ ID NO: 1;
(ii) the nucleotide sequence of the reverse primer is provided by SEQ ID NO: 17;
(iii) the nucleotide sequence of the probe is provided by any SEQ ID NO: 7 or 8; and/or
(iv) the nucleotide sequence of the PNA blocker is provided by SEQ ID NO: 22 or 23.

63.-67. (canceled)

68. The method of claim 54, wherein (i) the nucleotide sequence of the forward primer is provided by SEQ ID NO: 4;
(ii) the nucleotide sequence of the reverse primer is provided by SEQ ID NO: 19;
(iii) the nucleotide sequence of the probe is provided by SEQ ID NO: 14; and/or
(iv) the nucleotide sequence of the PNA blocker is provided by SEQ ID NO: 25.

69.-73. (canceled)

74. The method of claim 54, wherein (i) the nucleotide sequence of the forward primer is provided by SEQ ID NO: 5;
(ii) the nucleotide sequence of the reverse primer is provided by SEQ ID NO: 20;
(iii) the nucleotide sequence of the probe is provided by SEQ ID NO: 15; and/or
(iv) the nucleotide sequence of the PNA blocker is provided by SEQ ID NOs: 26.

75.-80. (canceled)

81. The method of claim 34, wherein the presence of the mutant allele in the sample indicates presence of the cancer in the subject and/or genotype of the cancer in the subject.

82. The method of claim 81, further comprising selecting a treatment regimen based on the presence of the cancer and/or genotype of the cancer.

83. A method for diagnosis and treatment of a cancer in a subject, comprising
performing a surgical procedure on the subject;
obtaining a sample from the subject; and
determining whether the sample contains one or more mutations in a target nucleotide sequence in the sample;
wherein if the sample is determined to contain the one or more mutations, locally administering an agent with mutation selectivity to the subject during the surgical procedure.

84.-90. (canceled)

91. A nucleic acid comprising the nucleotide sequence provided by any one of SEQ ID NOs: 1-26.

92. A kit, wherein
(a) the kit is for the rapid detection of IDH1 variants and comprises
(i) a forward primer comprising a nucleotide sequence provided by SEQ ID NO: 2 or 3;
(ii) a reverse primer comprising a nucleotide sequence provided by SEQ ID NO: 18;
(iii) a probe comprising a nucleotide sequence provided by any one of SEQ ID NOs: 9-13, and
(iv) a peptide nucleic acid (PNA) blocker comprising a nucleotide sequence provided by SEQ ID NO: 24; or
(b) the kit is for the rapid detection of TERT promoter variants and comprises
(i) a forward primer comprising a nucleotide sequence provided by SEQ ID NO: 1;
(ii) a reverse primer comprising a nucleotide sequence provided by SEQ ID NO: 17;
(iii) a probe comprising a nucleotide sequence provided by SEQ ID NO: 7 or 8, and
(iv) a peptide nucleic acid (PNA) blocker comprising a nucleotide sequence provided by SEQ ID NO: 22 or 23; or
(c) the kit is for the rapid detection of H3F3A variants and comprises
(i) a forward primer comprising a nucleotide sequence provided by SEQ ID NO: 4;
(ii) a reverse primer comprising a nucleotide sequence provided by SEQ ID NO: 19;
(iii) a probe comprising a nucleotide sequence provided by SEQ ID NO: 14, and
(iv) a peptide nucleic acid (PNA) blocker comprising a nucleotide sequence provided by SEQ ID NO: 25; or
(d) the kit is for the rapid detection of BRAF variants and comprises
(i) a forward primer comprising a nucleotide sequence provided by SEQ ID NO: 5;
(ii) a reverse primer comprising a nucleotide sequence provided by SEQ ID NO: 20;
(iii) a probe comprising a nucleotide sequence provided by SEQ ID NO: 15; and
(iv) a peptide nucleic acid (PNA) blocker comprising a nucleotide sequence provided by SEQ ID NO: 26.

93.-95. (canceled)

96. The kit of claim 92, further comprising instructions to locally administer a therapeutic agent to a subject if a mutant allele is detected.

97.-99. (canceled)
